(19) United States
(12) Patent Application Publication
(10) Pub. No.: US 2025/0169359 A1
(43) Pub. Date: May 22, 2025

(54) ORGANIC COMPOUND AND ORGANIC LIGHT-EMITTING DEVICE

(71) Applicant: CANON KABUSHIKI KAISHA, Tokyo (JP)

(72) Inventors: HIROKI OHRUI, Tokyo (JP); TAKAYOSHI KIKUCHI, Kanagawa (JP); YOSUKE NISHIDE, Kanagawa (JP); HIRONOBU IWAWAKI, Kanagawa (JP)

(21) Appl. No.: 18/946,747

(22) Filed: Nov. 13, 2024

(30) Foreign Application Priority Data
Nov. 21, 2023 (JP) 2023-197267

(51) Int. Cl. H10K 85/60; C07D 403/10; C07D 407/14; C07D 409/14; C09K 11/02; H10K 50/12; H10K 50/13

(52) U.S. Cl. CPC H10K 85/6572; C07D 403/10; C07D 407/14; C07D 409/14; C09K 11/02; H10K 85/615; H10K 85/654; H10K 85/6574; H10K 85/6576; H10K 50/12; H10K 50/13

(57) ABSTRACT

An organic compound represented by the following formula 1 or 2, which has high efficiency and durability.

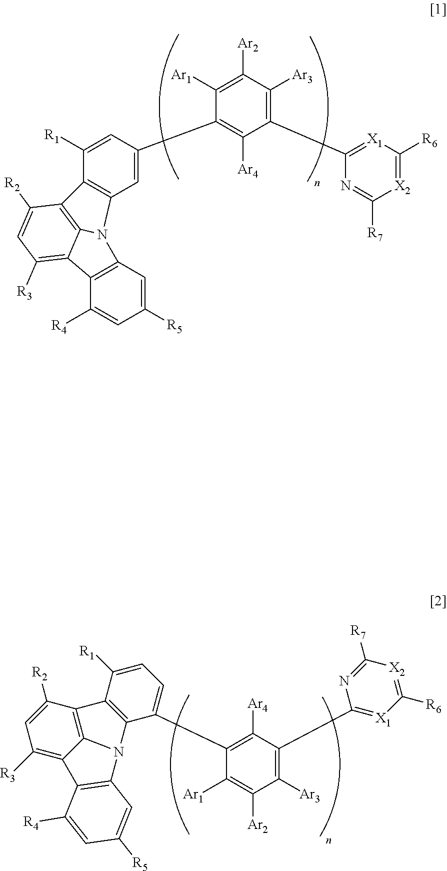

R₁ to R₇ are each independently selected from the group consisting of a hydrogen atom, a deuterium atom, and a halogen atom. Ar₁ to Ar₄ are each independently any of a hydrogen atom, a deuterium atom, or a substituted or unsubstituted alkyl group. One of x₁ and x₂ is a N atom, and the other is a C atom. n is an integer of 2 or more, and adjacent phenylene groups may be bonded together to form a ring.

### BACKGROUND OF THE INVENTION

### Field of the Invention

The present disclosure relates to an organic compound and an organic light-emitting device including the organic compound.

### Description of the Related Art

An organic light-emitting device (hereinafter sometimes referred to as an “organic electroluminescent device” or an “organic EL device”) is an electronic device that includes a pair of electrodes and an organic compound layer between the electrodes. Electrons and holes are injected from the pair of electrodes to generate an exciton of a light-emitting organic compound in the organic compound layer. When the exciton returns to its ground state, the organic light-emitting device emits light. With recent significant advances in organic light-emitting devices, it is characteristically possible to realize low drive voltage, various emission wavelengths, high-speed responsivity, thin and lightweight light-emitting devices, and the like.

Compounds suitable for organic light-emitting devices have been actively developed. This is because a compound that provides a device with good lifetime characteristics is important for high-performance organic light-emitting devices. Among the compounds that have been developed so far, an indolocarbazole derivative 1-a is disclosed in Korean patent No. 10-1891917 (hereinafter PTL 1), and indolocarbazole derivatives 1-b and 1-c are disclosed in Korean patent No. 10-2010397 (hereinafter PTL 2).

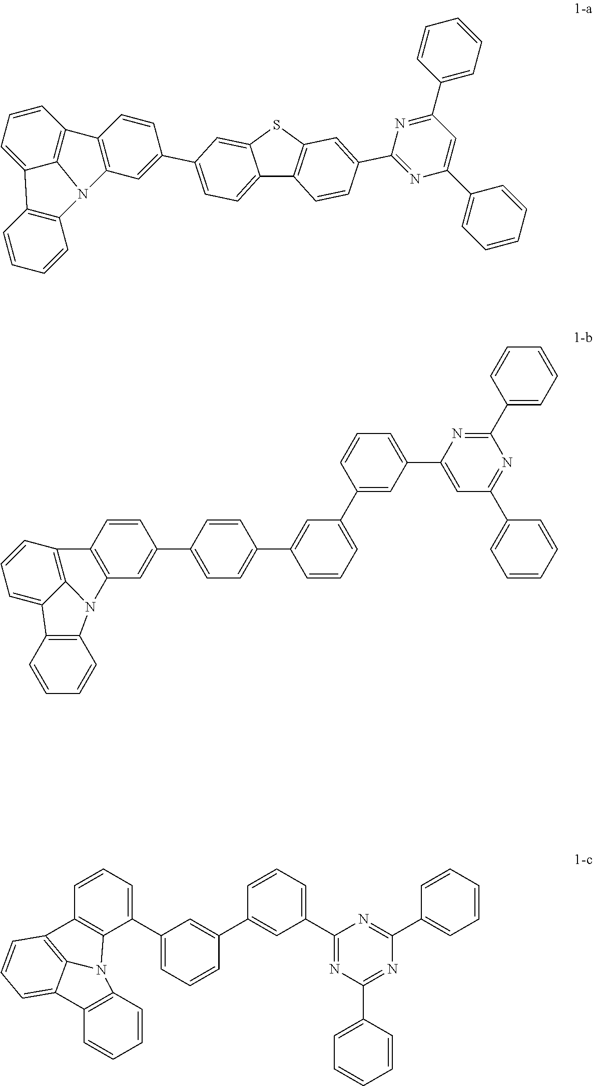 [SMILES: c1ccc(-c2cc(-c3ccccc3)nc(-c3ccc4c(c3)sc3cc(-c5ccc6c7cccc8c9ccccc9n(c6c5)c87)ccc34)n2)cc1 ; c1ccc(-c2cc(-c3cccc(-c4cccc(-c5ccc(-c6ccc7c8cccc9c%10ccccc%10n(c7c6)c98)cc5)c4)c3)nc(-c3ccccc3)n2)cc1 ; c1ccc(-c2nc(-c3ccccc3)nc(-c3cccc(-c4cccc(-c5cccc6c7cccc8c9ccccc9n(c56)c87)c4)c3)n2)cc1]

However, organic light-emitting device examples containing the compound 1-a described in PTL 1 and organic light-emitting device examples containing the compounds 1-b and 1-c described in PTL 2 are all desired to be further improved in light emission efficiency and durability.

### SUMMARY OF THE INVENTION

In view of the above disadvantages, the present disclosure provides an organic compound and an organic light-emitting device with high efficiency and durability.

The organic compound according to the present disclosure is represented by the following general formula [1] or [2].

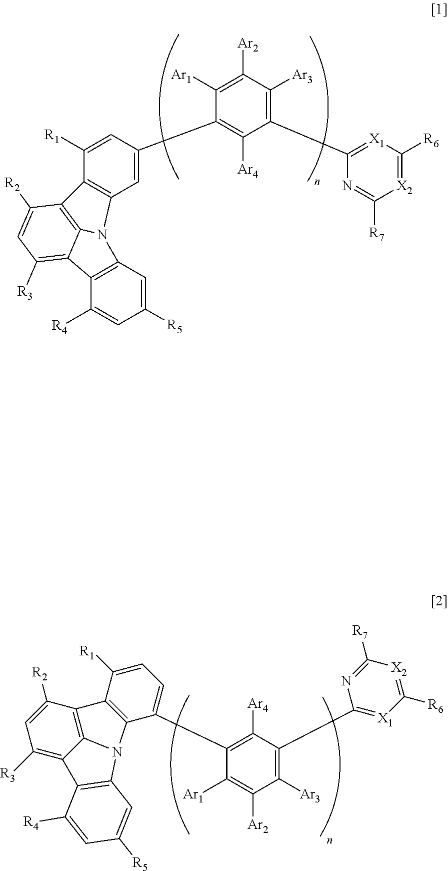

In the general formulae [1] and [2], R₁ to R₇ are each independently selected from the group consisting of a hydrogen atom, a deuterium atom, a halogen atom, a substituted or unsubstituted alkyl group, a substituted or unsubstituted aryl group, a substituted or unsubstituted heterocyclic group, a substituted or unsubstituted silyl group, a substituted or unsubstituted amino group, a cyano group, and a substituted or unsubstituted alkoxy group.

Ar₁ to Ar₄ are each independently selected from the group consisting of a hydrogen atom, a deuterium atom, a substituted or unsubstituted alkyl group, a substituted or unsubstituted aryl group, and a substituted or unsubstituted heterocyclic group.

One of x₁ and x₂ is a N atom, and the other is a C atom. n is an integer of 2 or more, and adjacent phenylene groups may be bonded together to form a ring.

Further features of the present disclosure will become apparent from the following description of exemplary embodiments with reference to the attached drawings.

### BRIEF DESCRIPTION OF THE DRAWINGS

FIG. 1 is a diagram showing the results of visualizing the orbital distributions of HOMO and LUMO of a compound.

FIG. 2 is a diagram showing the results of visualizing the orbital distributions of HOMO and LUMO of a compound.

FIG. 3A is a schematic cross-sectional view of an example of a pixel of a display apparatus according to an embodiment of the present disclosure. FIG. 3B is a schematic cross-sectional view of an example of a display apparatus including an organic light-emitting device according to an embodiment of the present disclosure.

FIG. 4 is a schematic view of an example of a display apparatus according to an embodiment of the present disclosure.

FIG. 5A is a schematic view of an example of an imaging apparatus according to an embodiment of the present disclosure. FIG. 5B is a schematic view of an example of electronic equipment according to an embodiment of the present disclosure.

FIG. 6A is a schematic view of an example of a display apparatus according to an embodiment of the present disclosure. FIG. 6B is a schematic view of an example of a foldable display apparatus.

FIG. 7A is a schematic view of an example of a lighting apparatus according to an embodiment of the present disclosure. FIG. 7B is a schematic view of an example of a moving body with a vehicle lamp according to an embodiment of the present disclosure.

FIG. 8A is a schematic view of an example of a wearable device according to an embodiment of the present disclosure. FIG. 8B is a schematic view of another example of a wearable device according to an embodiment of the present disclosure.

FIG. 9A is a schematic view of an example of an image-forming apparatus according to an embodiment of the present disclosure. FIG. 9B and FIG. 9C are each a schematic view of an example of an exposure light source for an image-forming apparatus according to an embodiment of the present disclosure.

### DESCRIPTION OF THE EMBODIMENTS

### (1) Organic Compound

An organic compound according to the present embodiment is a compound represented by the following general formula [1] or [2].

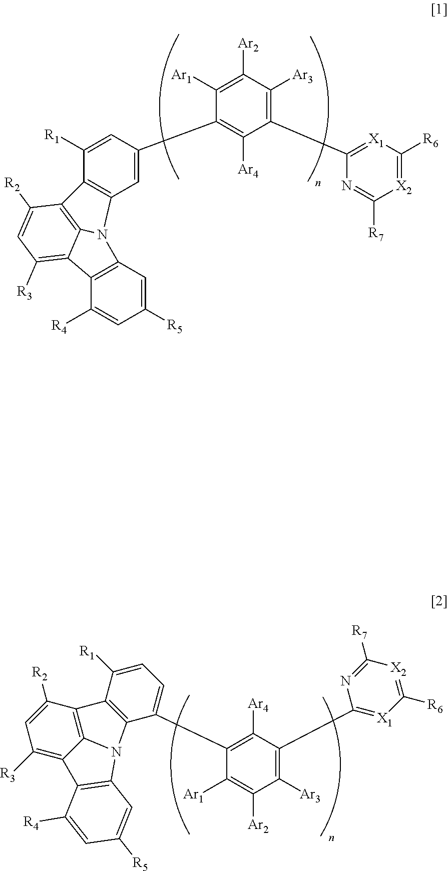

<R₁ to R₇>

In the general formulae [1] and [2], R₁ to R₇ are each independently selected from the group consisting of a hydrogen atom, a deuterium atom, a halogen atom, a substituted or unsubstituted alkyl group, a substituted or unsubstituted aryl group, a substituted or unsubstituted heterocyclic group, a substituted or unsubstituted silyl group, a substituted or unsubstituted amino group, a cyano group, and a substituted or unsubstituted alkoxy group.

At least one of R₁ to R₅, at least one of R₃ to R₅, or R₅ can be a group selected from the group consisting of a deuterium atom, a substituted or unsubstituted alkyl group, a substituted or unsubstituted aryl group, and a substituted or unsubstituted heterocyclic group. R₆ and R₇ can be a substituted or unsubstituted aryl group or a substituted or unsubstituted phenyl group.

The halogen atom is, for example, but not limited to, fluorine, chlorine, bromine, iodine, astatine, tennessine, or the like.

The alkyl group may be an alkyl group with 1 or more and 20 or less carbon atoms. The alkyl group is, for example, but not limited to, a methyl group, an ethyl group, a n-propyl group, an isopropyl group, a n-butyl group, a tert-butyl group, a sec-butyl group, an octyl group, a cyclohexyl group, a tert-pentyl group, a 3-methylpentan-3-yl group, a 1-adamantyl group, a 2-adamantyl group, or the like.

The aryl group may be an aryl group with 6 or more and 20 or less carbon atoms. The aryl group is, for example, but not limited to, a phenyl group, a naphthyl group, an indenyl group, a biphenyl group, a terphenyl group, a fluorenyl group, a phenanthryl group, a triphenylenyl group, a pyrenyl group, an anthranyl group, a perylenyl group, a chrysenyl group, or a fluoranthenyl group.

The heterocyclic group may be a heteroaryl group with 3 or more and 20 or less carbon atoms. The heterocyclic group is, for example, but not limited to, a pyridyl group, a pyrimidyl group, a pyrazyl group, a triazyl group, a benzofuranyl group, a benzothiophenyl group, a dibenzofuranyl group, a dibenzothiophenyl group, an oxazolyl group, an oxadiazolyl group, a thiazolyl group, a thiadiazolyl group, a carbazolyl group, an acridinyl group, or a phenanthrolyl group.

The silyl group is, for example, but not limited to, a trimethylsilyl group, a triphenylsilyl group, or the like.

The amino group is, for example, but not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, an N-piperidyl group, a carbazolyl group, an acridyl group, a trimethylamino group, a triphenylamino group, or the like.

The alkoxy group may be an alkoxy group with 1 or more and 10 or less carbon atoms. The alkoxy group is, for example, but not limited to, a methoxy group, an ethoxy group, a propoxy group, an isopropoxy group, a tert-butoxy group, a 2-ethyl-octyloxy group, a benzyloxy group, or the like.

An optional substituent of the alkyl group, the aryl group, the heterocyclic group, the silyl group, the amino group, or the alkoxy group is, for example, but not limited to, deuterium, an alkyl group, such as a methyl group, an ethyl group, a n-propyl group, an isopropyl group, a n-butyl group, or a tert-butyl group, an aralkyl group, such as a benzyl group, an aryl group, such as a phenyl group or a biphenyl group, a heterocyclic group, such as a pyridyl group or a pyrrolyl group, an amino group, such as a dimethylamino group, a diethylamino group, a dibenzylamino group, a diphenylamino group, or a ditolylamino group, an alkoxy group, such as a methoxy group, an ethoxy group, or a propoxy group, an aryloxy group, such as a phenoxy group, a halogen atom, such as fluorine, chlorine, bromine, or iodine, a cyano group, or the like.

<Ar₁ to Ar₄>

In the general formulae [1] and [2], Ar₁ to Ar₄ are each independently any of a hydrogen atom, a deuterium atom, a substituted or unsubstituted alkyl group, a substituted or unsubstituted aryl group, and a substituted or unsubstituted heterocyclic group. Ar₁ to Ar₄ can be each independently selected from the group consisting of a hydrogen atom, a deuterium atom, a substituted or unsubstituted aryl group, and a substituted or unsubstituted heterocyclic group. Ar₁ to Ar₄ are each independently any of a hydrogen atom and a substituted or unsubstituted aryl group or any of a hydrogen atom and a substituted or unsubstituted phenyl group.

Ar₁s, Ar₂s, Ar₃s, or Ar₄s bonded to different phenylene rings may be identical to or different from each other.

An optional substituent of the alkyl group, the aryl group, and the heterocyclic group is, for example, but not limited to, one of those described for R₁ to R₇.

<x₁, x₂>

In the general formulae [1] and [2], one of x₁ and x₂ is a N atom, and the other is a C atom.

<n>

In the general formulae [1] and [2], n is an integer of 2 or more, preferably an integer of 2 or more and 4 or less.

Adjacent phenylene groups may be bonded together to form a ring. A compound with adjacent phenylene groups bonded to each other to form a ring is, for example, but not limited to, a compound represented by the general formula [6] or [7] or the like described later.

### <Preferred Compound>

Although a preferred organic compound according to the present embodiment is described below, the present embodiment is not limited thereto.

### [Organic Compounds Represented by General Formulae [3] to [5]]

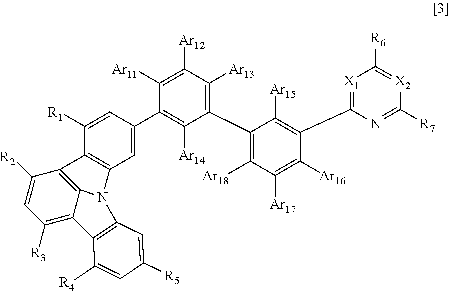

In the general formula [3], Ar₁₁ to Ar₁₈ are each independently selected from the group consisting of a hydrogen atom, a deuterium atom, a substituted or unsubstituted aryl group, and a substituted or unsubstituted heterocyclic group.

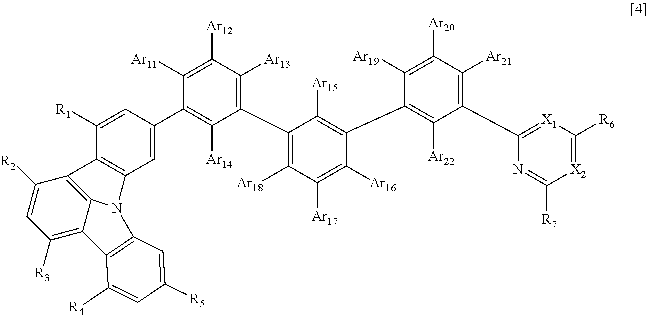

In the general formula [4], Ar₁₁ to Ar₂₂ are each independently selected from the group consisting of a hydrogen atom, a deuterium atom, a substituted or unsubstituted aryl group, and a substituted or unsubstituted heterocyclic group.

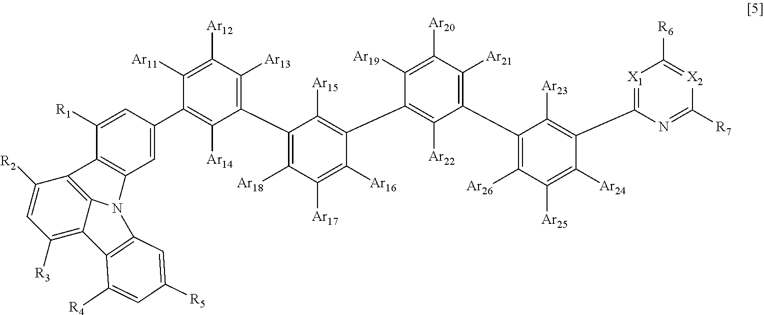

In the general formula [5], Ar₁₁ to Ar₂₆ are each independently selected from the group consisting of any of a hydrogen atom, a deuterium atom, a substituted or unsubstituted aryl group, and a substituted or unsubstituted heterocyclic group.

In the general formulae [3] to [5], the aryl group and the heterocyclic group represented by Ar₁₁ to Ar₂₆ and an optional substituent of these groups are, for example, but not limited to, the groups described for R₁ to R₇. Ar₁₁ to Ar₂₆ are each independently selected from the group consisting of a hydrogen atom and a substituted or unsubstituted aryl group or any of a hydrogen atom and a substituted or unsubstituted phenyl group. Adjacent phenylene groups may not be bonded together to form a ring.

### [Organic Compounds Represented by General Formulae [6] and [7]]

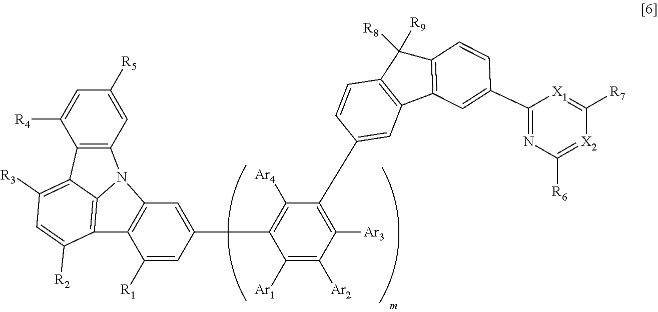

In the general formula [6], R₈ and R₉ are each independently selected from the group consisting of a hydrogen atom, a deuterium atom, a substituted or unsubstituted alkyl group, a substituted or unsubstituted aryl group, and a substituted or unsubstituted heterocyclic group. m is 1 or 2.

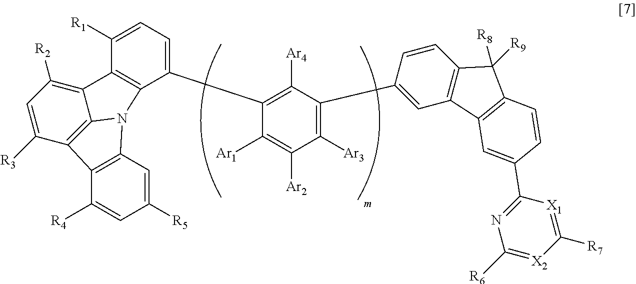

In the general formula [7], R₈ and R₉ are each independently selected from the group consisting of a hydrogen atom, a deuterium atom, a substituted or unsubstituted alkyl group, a substituted or unsubstituted aryl group, and a substituted or unsubstituted heterocyclic group. m is 1 or 2.

In the general formulae [6] and [7], the alkyl group, the aryl group, and the heterocyclic group represented by R₈ and R₉ and an optional substituent of these groups are, for example, but not limited to, the groups described for R₁ to R₇. R₈ and R₉ can be a substituted or unsubstituted alkyl group or a substituted or unsubstituted methyl group. A phenylene group other than the phenylene group forming the fluorenylene skeleton may not be bonded to an adjacent phenylene group to form a ring.

### <Characteristics>

The organic compound represented by the general formula [1] or [2] has the following characteristics.

(1-1) Since all the phenylene groups constituting the linking group for bonding the indolocarbazole skeleton and the pyrimidine skeleton are bonded at the meta position, the organic compound has high T₁ (lowest excited triplet energy) and low ΔST, resulting in high efficiency, a low voltage, and high durability.
(1-2) HOMO and LUMO are separated from each other, which results in improved stability to electrons and high durability.
(1-3) Since all the phenylene groups constituting the linking group for bonding the indolocarbazole skeleton and the pyrimidine skeleton are bonded at the meta position, the organic compound has a low HOMO level and has high durability due to high stability to oxygen.
(1-4) The heterocycle bonded to the indolocarbazole skeleton via the linking group is the pyrimidyl group, which results in improved dipole moment and high durability.

These characteristics are described below.

(1-1) Since all the phenylene groups constituting the linking group for bonding the indolocarbazole skeleton and the pyrimidine skeleton are bonded at the meta position, the organic compound has high T₁ (lowest excited triplet energy) and low ΔST, resulting in high efficiency, a low voltage, and high durability.

In the organic compound according to the present embodiment, the present inventors have found that when all the phenylene groups constituting the linking group for bonding the indolocarbazole skeleton and the pyrimidine skeleton are bonded at the meta position, the organic compound has high T₁ and low ΔST.

Table 1 shows the calculated values of S₁ and T₁ of exemplary compounds A1 and B2, which are organic compounds according to the present embodiment, and comparative compounds 1-a and 1-b and ΔST calculated therefrom. The comparative compound 1-a is a compound described in Patent Literature 1, and the comparative compound 1-b is a compound described in Patent Literature 2.

The calculation was performed using molecular orbital calculation. The calculation method in the molecular orbital calculation method utilized a widely used density functional theory (DFT). B3LYP was used as the functional, and 6-31G* was used as the basis function. The molecular orbital calculation method was performed using widely used Gaussian 09 (Gaussian 09, Revision C. 01, M. J. Frisch, G. W. Trucks, H. B. Schlegel, G. E. Scuseria, M. A. Robb, J. R. Cheeseman, G. Scalmani, V. Barone, B. Mennucci, G. A. Petersson, H. Nakatsuji, M. Caricato, X. Li, H. P. Hratchian, A. F. Izmaylov, J. Bloino, G. Zheng, J. L. Sonnenberg, M. Hada, M. Ehara, K. Toyota, R. Fukuda, J. Hasegawa, M. Ishida, T. Nakajima, Y. Honda, O. Kitao, H. Nakai, T. Vreven, J. A. Montgomery, Jr., J. E. Peralta, F. Ogliaro, M. Bearpark, J. J. Heyd, E. Brothers, K. N. Kudin, V. N. Staroverov, T. Keith, R. Kobayashi, J. Normand, K. Raghavachari, A. Rendell, J. C. Burant, S. S. Iyengar, J. Tomasi, M. Cossi, N. Rega, J. M. Millam, M. Klene, J. E. Knox, J. B. Cross, V. Bakken, C. Adamo, J. Jaramillo, R. Gomperts, R. E. Stratmann, O. Yazyev, A. J. Austin, R. Cammi, C. Pomelli, J. W. Ochterski, R. L. Martin, K. Morokuma, V. G. Zakrzewski, G. A. Voth, P. Salvador, J. J. Dannenberg, S. Dapprich, A. D. Daniels, O. Farkas, J. B. Foresman, J. V. Ortiz, J. Cioslowski, and D. J. Fox, Gaussian, Inc., Wallingford CT, 2010).

TABLE 1

| | | T₁ | ΔST | S₁ |
|---|---|---|---|---|
| | Structure | (ev) | (ev) | (ev) |
| Compound A1 | 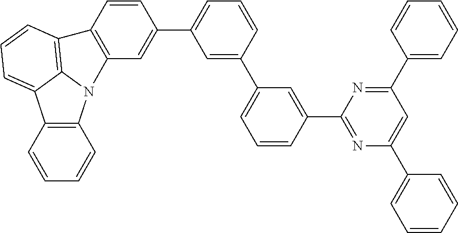 [SMILES: c1ccc(-c2cc(-c3ccccc3)nc(-c3cccc(-c4cccc(-c5ccc6c7cccc8c9ccccc9n(c6c5)c87)c4)c3)n2)cc1] | 2.83 | 0.76 | 3.59 |
| Compound B2 | 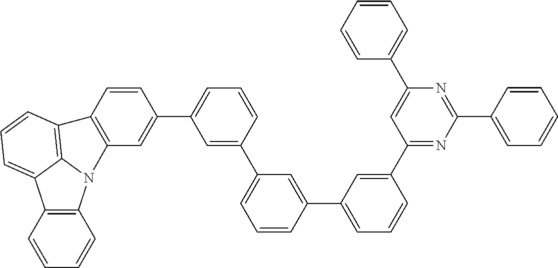 [SMILES: c1ccc(-c2cc(-c3cccc(-c4cccc(-c5cccc(-c6ccc7c8cccc9c%10ccccc%10n(c7c6)c98)c5)c4)c3)nc(-c3ccccc3)n2)cc1] | 2.83 | 0.76 | 3.59 |
| Comparative Compound 1-a | 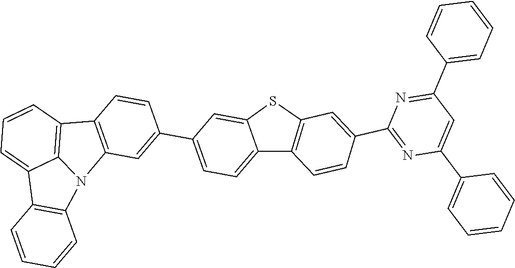 [SMILES: c1ccc(-c2cc(-c3ccccc3)nc(-c3ccc4c(c3)sc3cc(-c5ccc6c7cccc8c9ccccc9n(c6c5)c87)ccc34)n2)cc1] | 2.58 | 0.83 | 3.42 |
| Comparative Compound 1-b | 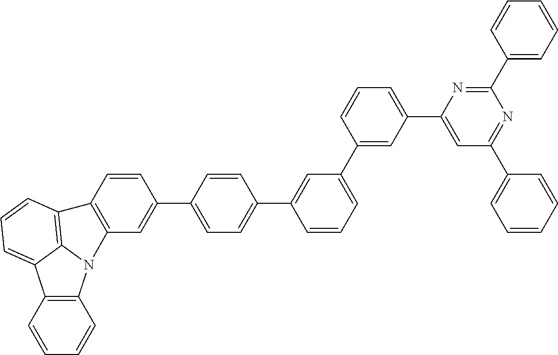 [SMILES: c1ccc(-c2cc(-c3cccc(-c4cccc(-c5ccc(-c6ccc7c8cccc9c%10ccccc%10n(c7c6)c98)cc5)c4)c3)nc(-c3ccccc3)n2)cc1] | 2.75 | 0.80 | 3.55 |

Table 1 shows that the exemplary compounds A1 and B2 have a T₁ of 2.83 ev, whereas the comparative compound 1-a has a T₁ of 2.58 ev, and the comparative compound 1-b has a T₁ of 2.75 ev. The exemplary compounds A1 and B2 in which all the phenylene groups constituting the linking group for bonding the indolocarbazole skeleton and the pyrimidine skeleton are bonded at the meta position have a higher T₁ by 0.3 ev than the comparative compound 1-a in which the linking group is a dibenzothiophene ring. The comparative compound 1-b, which is closer in structure to the compound of the present embodiment than the comparative compound 1-a, has a structure in which one of three phenylene groups constituting the linking group is bonded at the para position. It can be seen that the exemplary compounds A1 and B2 have a higher T₁ by 0.08 ev than the comparative compound 1-b.

It can also be seen that the exemplary compounds A1 and B2 have the lowest ΔST and particularly have a lower ΔST than the comparative compound 1-b, which has S₁ closer to S₁ of the exemplary compounds A1 and B2.

The effect of low ΔST is described below. Phosphorescent devices are organic light-emitting devices that use the T₁ energy for light emission. A host material used in a light-emitting layer of an organic light-emitting device can have higher T₁ energy than light-emitting materials that emit phosphorescence. This is because higher T₁ results in higher energy transfer efficiency to a guest molecule, improved device efficiency, shorter exciton lifetime, and improved device durability.

On the other hand, organic compounds with higher T₁ energy tend to have higher S₁ energy. Higher S₁ energy means a larger band gap.

In the present description, the band gap refers to an energy difference between the highest occupied molecular orbital (HOMO) and the lowest unoccupied molecular orbital (LUMO).

A large band gap of a host material in a light-emitting layer results in lower injectability of holes and electrons from a peripheral layer to the light-emitting layer. This unfavorably increases the voltage of the device and shortens the device lifetime due to unnecessary charge accumulation. On the other hand, a small band gap of a host material in a light-emitting layer results in improved injectability of holes and electrons from a peripheral layer to the light-emitting layer. This can lower the voltage of the device and reduce the decrease in the device lifetime due to unnecessary charge accumulation. Thus, an organic compound with high T₁ energy and low S₁ energy, that is, with low ΔST, can be used as a host material in a light-emitting layer of an organic light-emitting device.

The compound according to the present embodiment, which has low ΔST, is a material that is designed to have high T₁ but can have controllable S₁ and have both high efficiency and a lower voltage.

(1-2) HOMO and LUMO are separated from each other, which results in improved stability to electrons and high durability.

The organic compound according to the present embodiment can be used in a hole transport layer, an electron-blocking layer, a light-emitting layer, another functional layer, and the like of an organic light-emitting device and, among them, can be suitably used as a host in the light-emitting layer. Furthermore, an organic compound according to the present disclosure has high T₁ and can therefore be suitably used as a host for a light-emitting layer in a system in which a triplet excited state is used for light emission, such as phosphorescence or delayed fluorescence.

The organic compound according to the present embodiment, which has the indolocarbazole skeleton, has high T₁, bonding stability, and hole transport properties. On the other hand, the indolocarbazole skeleton is unstable to electrons, and the orbital distribution of LUMO can therefore be present at a site different from that of the indolocarbazole skeleton. The organic compound according to the present embodiment has the pyrimidine skeleton with electron transport properties. The phenylene groups constituting the linking group to the indolocarbazole skeleton can be bonded at the meta position to distribute the LUMO only in the pyrimidine skeleton and only the HOMO in the indolocarbazole skeleton.

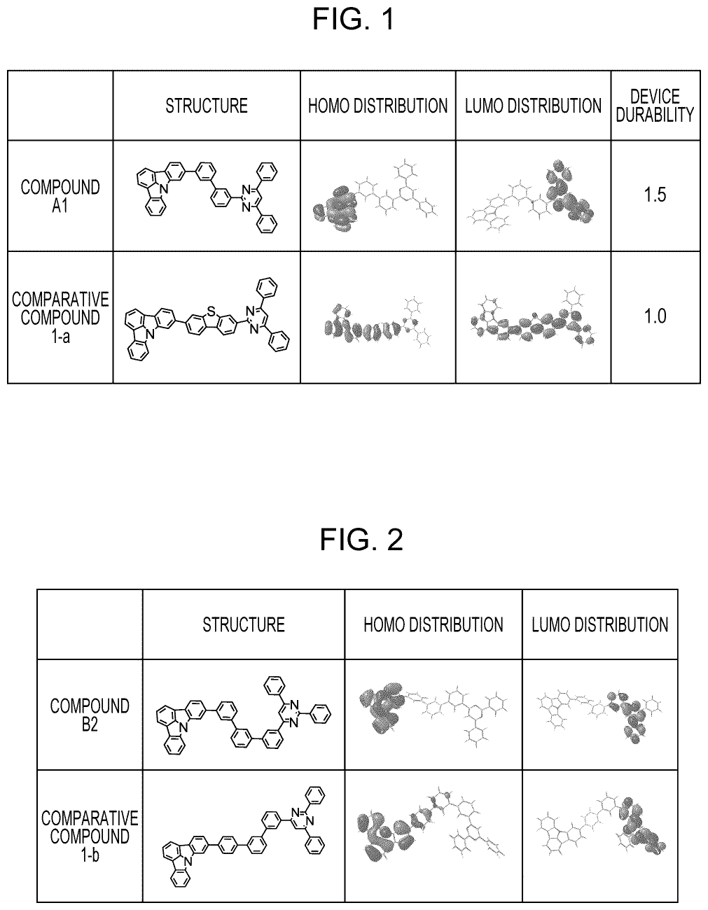

FIG. 1 shows the results of visualizing the orbital distributions of HOMO and LUMO of the exemplary compound A1 and the comparative compound 1-a of the present embodiment by the same calculation method as described above. As the device durability, FIG. 1 also shows the luminance decay rate ratio of an exemplary embodiment using the compound A1 as a host of an organic light-emitting device relative to the value (1.0) of a device using the comparative compound 1-a.

As illustrated in FIG. 1, in the exemplary compound A1, only the HOMO is distributed in the site of the indolocarbazole skeleton, and the LUMO is distributed in the pyrimidine skeleton. On the other hand, in the comparative compound 1-a, the LUMO is distributed not only in the pyrimidine skeleton but also in the indolocarbazole skeleton. Consequently, the comparative compound 1-a is unstable to electrons, resulting in the device with low durability, whereas the exemplary compound A1 has separated HOMO and LUMO, resulting in high durability.

(1-3) Since all the phenylene groups constituting the linking group for bonding the indolocarbazole skeleton and the pyrimidine skeleton are bonded at the meta position, the organic compound has a low HOMO level and has high durability due to high stability to oxygen.

First, a low HOMO level means a level far from the vacuum level. On the other hand, a high HOMO level means a level close to the vacuum level.

The comparative compound 1-b with a structure closer to the structure of the exemplary compound B2 of the present embodiment was selected as a comparative objective. Table 2 shows calculated values of HOMO, LUMO, and S₁ of these two compounds determined by the same calculation method as described above. As the device durability, Table 2 also shows the luminance decay rate ratio of an exemplary embodiment using the compound B2 as a host of an organic light-emitting device relative to the value (1.0) of a device using the comparative compound 1-b.

TABLE 2

| | | HOMO | LUMO | S₁ | Device |
|---|---|---|---|---|---|
| | Structure | (ev) | (ev) | (ev) | durability |
| Compound B2 | 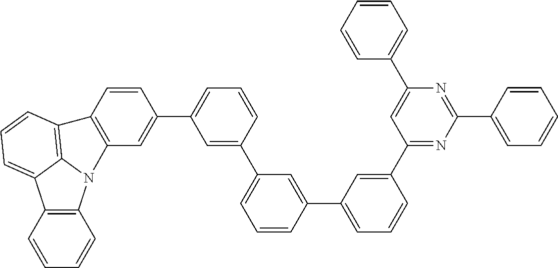 [SMILES: c1ccc(-c2cc(-c3cccc(-c4cccc(-c5cccc(-c6ccc7c8cccc9c%10ccccc%10n(c7c6)c98)c5)c4)c3)nc(-c3ccccc3)n2)cc1] | −5.78 | −1.44 | 3.59 | 1.2 |
| Comparative Compound 1-b | 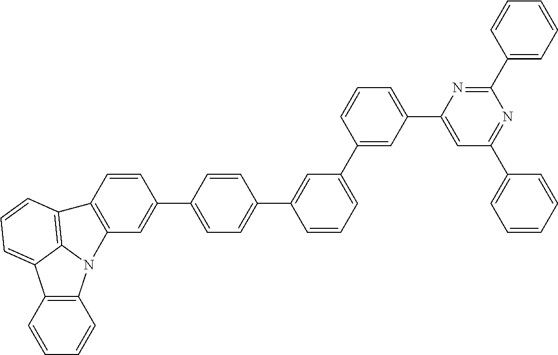 [SMILES: c1ccc(-c2cc(-c3cccc(-c4cccc(-c5ccc(-c6ccc7c8cccc9c%10ccccc%10n(c7c6)c98)cc5)c4)c3)nc(-c3ccccc3)n2)cc1] | −5.62 | −1.46 | 3.55 | 1.0 |

Table 2 shows that the exemplary compound B2 and the comparative compound 1-b have almost the same LUMO level, but the HOMO level is lower in the exemplary compound B2 by 0.16 ev than in the comparative compound 1-b. Since an oxidation reaction by oxygen is more likely to be affected as the HOMO level increases, the compound according to the present embodiment with a low HOMO level is stable to oxygen and consequently has high durability.

The difference in the HOMO level and in the LUMO level can be examined by visualizing each orbital distribution in the same manner as described above. FIG. 2 shows the results.

As illustrated in FIG. 2, although there is no difference in the orbital distribution of LUMO between the two compounds, the orbital distribution of HOMO is localized at the indolocarbazole skeleton in the exemplary compound B2. On the other hand, in the comparative compound 1-b, the HOMO is distributed up to two phenylene groups bonded to the indolocarbazole skeleton at the para position. Consequently, in the comparative compound 1-b, conjugation with the phenylene group bonded at the para position in the linking group increases the HOMO.

Unlike the comparative compound 1-b, the exemplary compound B2 has a structure in which all the phenylene groups of the linking group are bonded at the meta position, and has a structure in which the π conjugation of the phenylene groups is interrupted. Thus, the distribution of the HOMO is localized in the indolocarbazole skeleton, and the HOMO level can be kept low.

(1-4) The heterocycle bonded to the indolocarbazole skeleton via the linking group is the pyrimidyl group, which results in improved dipole moment and high durability.

In the organic compound represented by the general formula [1] or [2], the present inventors have paid attention to a terminal unit (a unit bonded to the indolocarbazole skeleton via the linking group) of the molecule and the permanent dipole moment.

A compound in an organic layer, particularly a light-emitting layer, of an organic light-emitting device transits repeatedly between the ground state and the excited state in the process of light emission of the organic light-emitting device. In particular, in an organic phosphorescent device, it is important to control the triplet excited state (T₁), which occupies 75% of the excited state. For example, energy should be efficiently transferred from T₁ of a host molecule to a guest molecule to efficiently emit light from the guest molecule. Low energy transfer efficiency results in an increase in probability that generated excitation energy is used for a reaction with an adjacent molecule, and lower durability due to the generation of a quencher molecule.

It is known that the energy transfer process from T₁ of a host molecule to a guest molecule occurs by Dexter energy transfer. To improve the efficiency of Dexter energy transfer, it is important to minimize the distance between a host molecule and a guest molecule. For this purpose, a heterocycle that suitably interacts with a metal atom (for example, an iridium atom) of a guest molecule can be provided at an end of the molecule to increase the intermolecular interaction with the guest molecule and reduce the intermolecular distance from the guest molecule.

An Ir complex, a Pt complex, or the like is a guest molecule with high polarity. Thus, to increase the interaction with such a molecule, a host molecule can have a high permanent dipole moment to increase the polarity. Due to a heteroatom in its skeleton, a heterocycle characteristically has increased polarization and polarity. As a result of intensive studies, the present inventors have found that a pyrimidyl group is suitable as a heterocycle at an end portion of the molecule opposite the indolocarbazole skeleton to reduce the intermolecular distance between a host molecule and a guest molecule.

Table 3 shows permanent dipole moments calculated by the molecular orbital calculation for the exemplary compound A1 and an exemplary compound E1 of the present embodiment and the comparative compound 1-c described in Patent Literature 2. As the device durability, Table 3 also shows the luminance decay rate ratio of exemplary embodiments using each compound as a host of an organic light-emitting device relative to the value (1.0) of a device using the comparative compound 1-c.

TABLE 3

| | | T₁ | Dipole moment | Device |
|---|---|---|---|---|
| | Structure | (ev) | (Debye) | durability |
| Compound A1 | 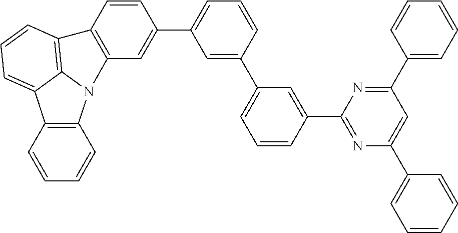 [SMILES: c1ccc(-c2cc(-c3ccccc3)nc(-c3cccc(-c4cccc(-c5ccc6c7cccc8c9ccccc9n(c6c5)c87)c4)c3)n2)cc1] | 2.83 | 2.7 | 1.3 |
| Compound E1 | 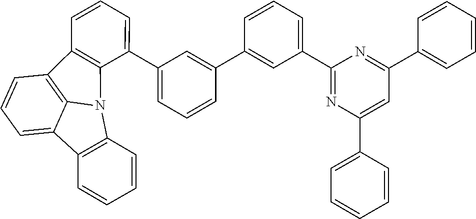 [SMILES: c1ccc(-c2cc(-c3ccccc3)nc(-c3cccc(-c4cccc(-c5cccc6c7cccc8c9ccccc9n(c56)c87)c4)c3)n2)cc1] | 2.94 | 2.2 | 1.3 |
| Comparative compound 1-C | 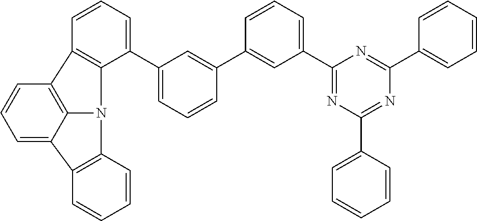 [SMILES: c1ccc(-c2nc(-c3ccccc3)nc(-c3cccc(-c4cccc(-c5cccc6c7cccc8c9ccccc9n(c56)c87)c4)c3)n2)cc1] | 2.96 | 0.8 | 1.0 |

Although all of the exemplary compounds A1 and E1 and the comparative compound 1-c have high T₁ due to the phenylene groups bonded at the meta position, unlike the compounds according to the present embodiment, the comparative compound 1-c has a low dipole moment due to a triazine skeleton at an end portion thereof. Unlike the pyrimidine skeleton, the triazine skeleton has a low dipole moment due to the nitrogen atoms isotropically arranged in the six-membered ring. On the other hand, the pyrimidine skeleton has a high dipole moment due to the nitrogen atoms anisotropically arranged in the six-membered ring. Thus, due to the pyrimidine skeleton at an end portion, the compound according to the present embodiment has an improved dipole moment as the entire molecule and increased molecular polarity. This increases the interaction with the guest molecule, efficiently transfers energy from the host to the guest, and improves the durability.

The organic compound according to the present embodiment can also have the following characteristics.

(1-5) When at least one of R₁ to R₅ is a group other than a hydrogen atom, the organic compound has high sublimability.

In the general formula [1] or [2], when at least one of R₁ to R₅ is a group other than a hydrogen atom, this results in a decrease in molecular symmetry, a decrease in molecular crystallinity, and therefore a decrease in sublimation temperature. This can also suppress overlap of fused rings between molecules and suppress crystallization of the molecules. Thus, at least one of R₁ to R₅ in the organic compound according to the present embodiment can be a group other than a hydrogen atom, that is, a group selected from a deuterium atom, a halogen atom, a substituted or unsubstituted alkyl group, a substituted or unsubstituted aryl group, a substituted or unsubstituted heterocyclic group, a substituted or unsubstituted silyl group, a substituted or unsubstituted amino group, a cyano group, and a substituted or unsubstituted alkoxy group. At least one of R₁ to R₅ can be a group selected from a deuterium atom, a substituted or unsubstituted alkyl group, a substituted or unsubstituted aryl group, and a substituted or unsubstituted heterocyclic group. In the presence of a group other than a hydrogen atom, the indolocarbazole skeleton with high planarity can have a substituent. More specifically, at least one of R₃, R₄, and R₅ can have a group other than a hydrogen atom, and particularly R₅ can have a group other than a hydrogen atom.

The group other than a hydrogen atom can be a group with a structure bulkier than a hydrogen atom. More specifically, the group other than a hydrogen atom can be a deuterium atom, an alkyl group with 1 to 4 carbon atoms, or an aryl group with 6 to 12 carbon atoms, or can be a methyl group, a tert-butyl group, or a substituted or unsubstituted phenyl group.

In particular, when the group other than a hydrogen atom is an alkyl group, the organic compound according to the present embodiment can further suppress the overlap of the indolocarbazole skeletons between its molecules and therefore has high sublimability and good film properties. Likewise, when the group other than a hydrogen atom is an aryl group, in particular, when R₅ is an aryl group, the aryl group can act as a steric hindrance group and further suppress the overlap of the indolocarbazole skeletons, resulting in high sublimability and good film properties. Even when R₅ is an aryl group, no conjugation with the HOMO occurs, so that the HOMO level does not increase, and the stability to oxygen is still stable.

### SPECIFIC EXAMPLES

Specific examples of the organic compound according to the present embodiment are described below. However, the present embodiment is not limited to these examples.

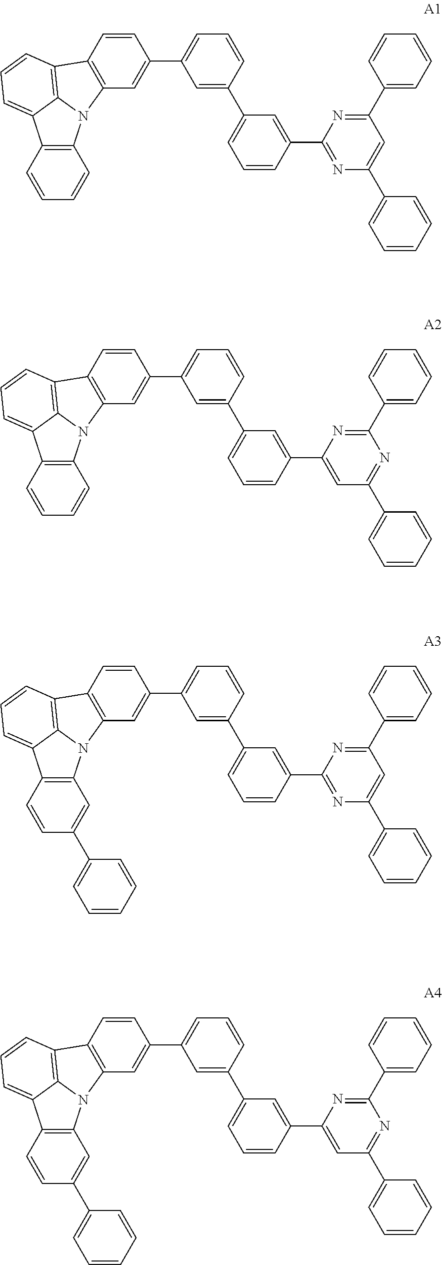 [SMILES: c1ccc(-c2cc(-c3ccccc3)nc(-c3cccc(-c4cccc(-c5ccc6c7cccc8c9ccccc9n(c6c5)c87)c4)c3)n2)cc1 ; c1ccc(-c2cc(-c3cccc(-c4cccc(-c5ccc6c7cccc8c9ccccc9n(c6c5)c87)c4)c3)nc(-c3ccccc3)n2)cc1 ; c1ccc(-c2ccc3c4cccc5c6ccc(-c7cccc(-c8cccc(-c9nc(-c%10ccccc%10)cc(-c%10ccccc%10)n9)c8)c7)cc6n(c3c2)c45)cc1 ; c1ccc(-c2ccc3c4cccc5c6ccc(-c7cccc(-c8cccc(-c9cc(-c%10ccccc%10)nc(-c%10ccccc%10)n9)c8)c7)cc6n(c3c2)c45)cc1]

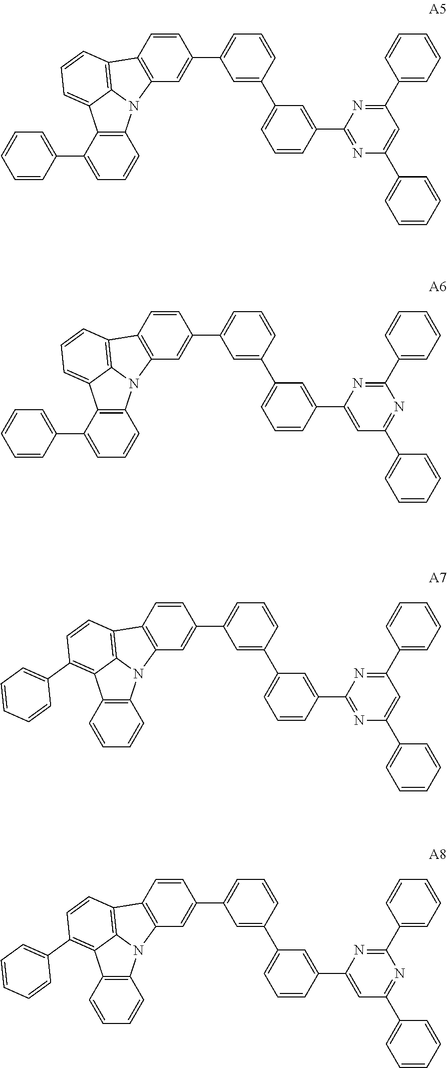 [SMILES: c1ccc(-c2cc(-c3ccccc3)nc(-c3cccc(-c4cccc(-c5ccc6c7cccc8c9c(-c%10ccccc%10)cccc9n(c6c5)c78)c4)c3)n2)cc1 ; c1ccc(-c2cc(-c3cccc(-c4cccc(-c5ccc6c7cccc8c9c(-c%10ccccc%10)cccc9n(c6c5)c78)c4)c3)nc(-c3ccccc3)n2)cc1 ; c1ccc(-c2cc(-c3ccccc3)nc(-c3cccc(-c4cccc(-c5ccc6c7ccc(-c8ccccc8)c8c9ccccc9n(c6c5)c78)c4)c3)n2)cc1 ; c1ccc(-c2cc(-c3cccc(-c4cccc(-c5ccc6c7ccc(-c8ccccc8)c8c9ccccc9n(c6c5)c78)c4)c3)nc(-c3ccccc3)n2)cc1]

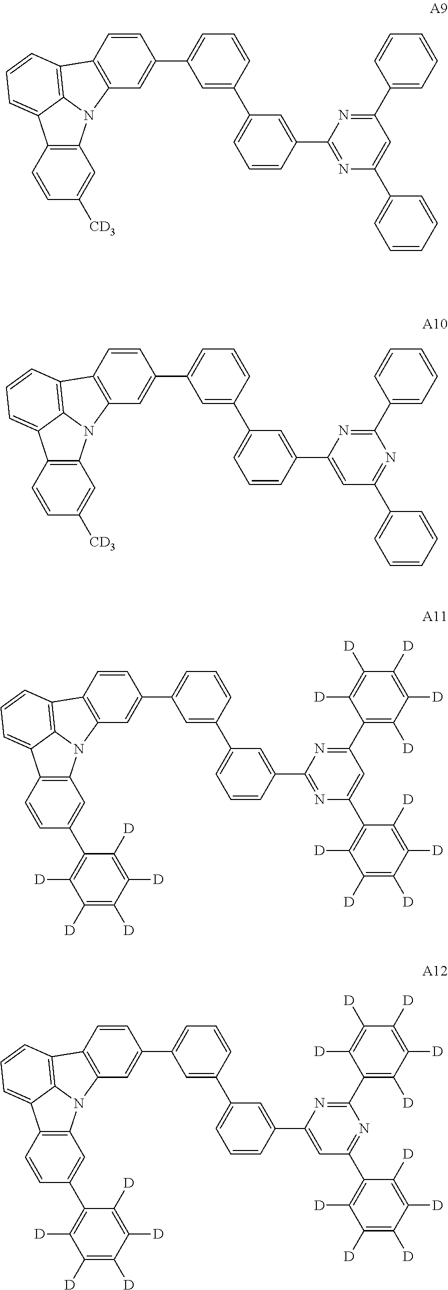

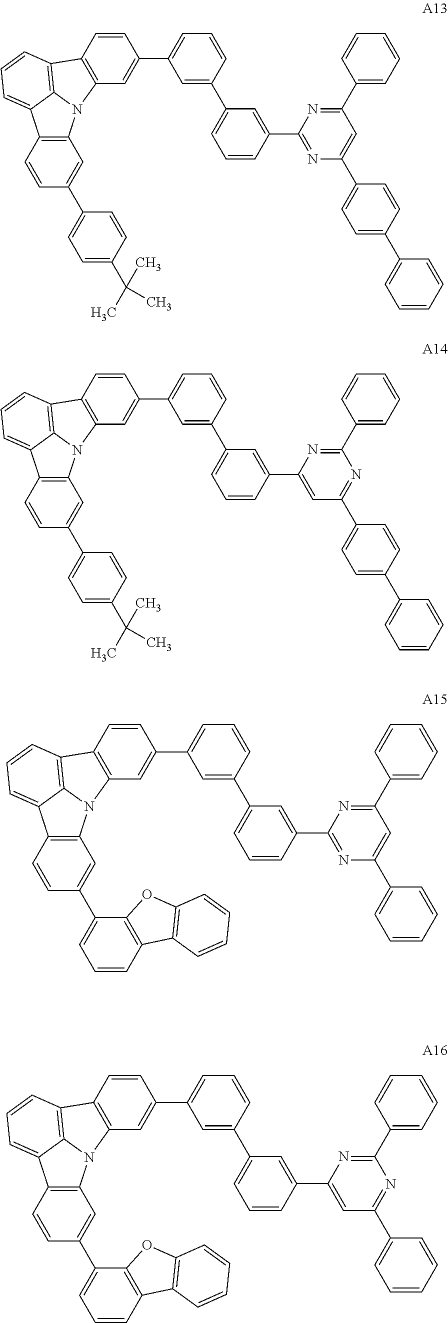

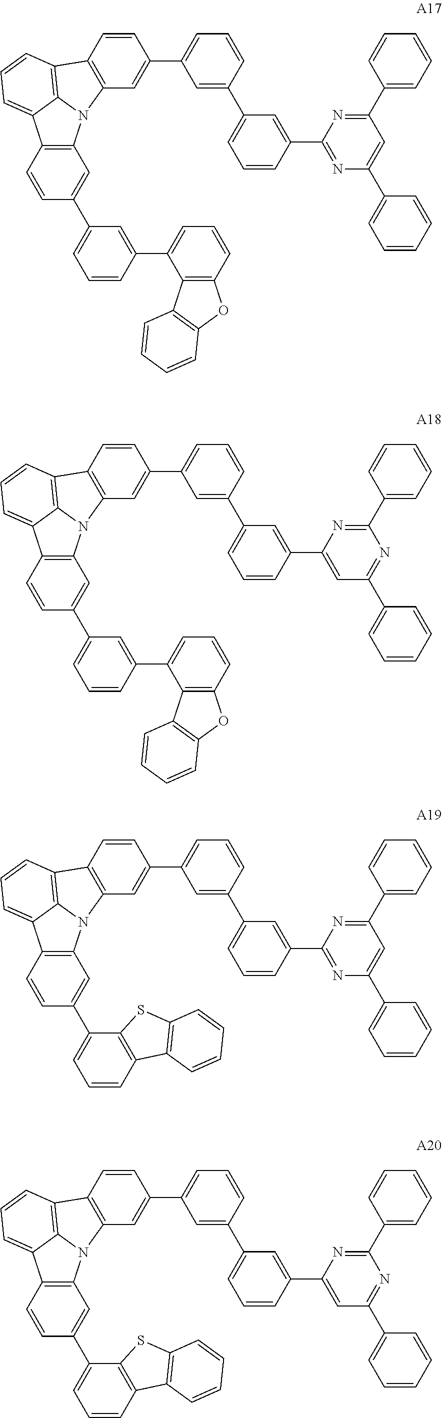

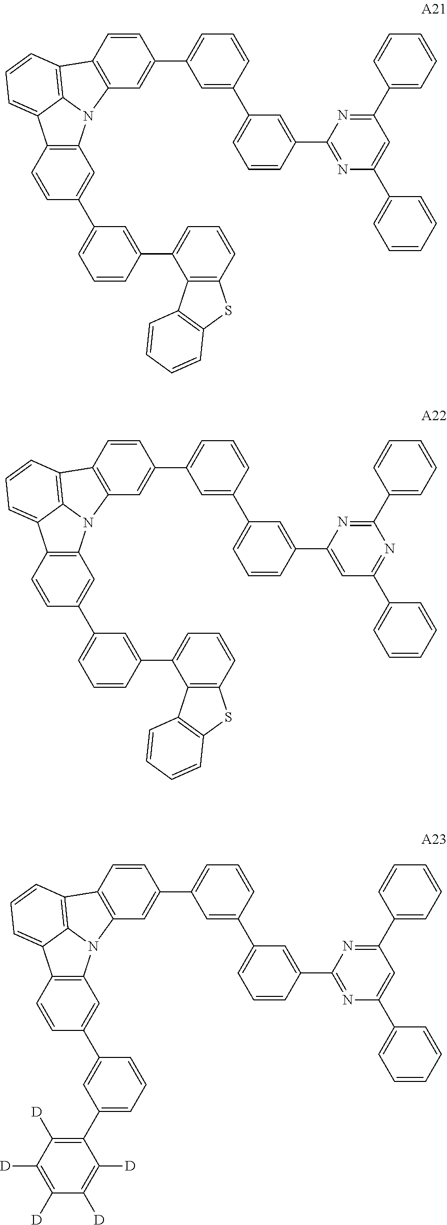 [SMILES: c1ccc(-c2cc(-c3ccccc3)nc(-c3cccc(-c4cccc(-c5ccc6c7cccc8c9ccc(-c%10cccc(-c%11cccc%12sc%13ccccc%13c%11%12)c%10)cc9n(c6c5)c78)c4)c3)n2)cc1 ; c1ccc(-c2cc(-c3cccc(-c4cccc(-c5ccc6c7cccc8c9ccc(-c%10cccc(-c%11cccc%12sc%13ccccc%13c%11%12)c%10)cc9n(c6c5)c78)c4)c3)nc(-c3ccccc3)n2)cc1 ; [2H]c1c([2H])c([2H])c(-c2cccc(-c3ccc4c5cccc6c7ccc(-c8cccc(-c9cccc(-c%10nc(-c%11ccccc%11)cc(-c%11ccccc%11)n%10)c9)c8)cc7n(c4c3)c65)c2)c([2H])c1[2H]]

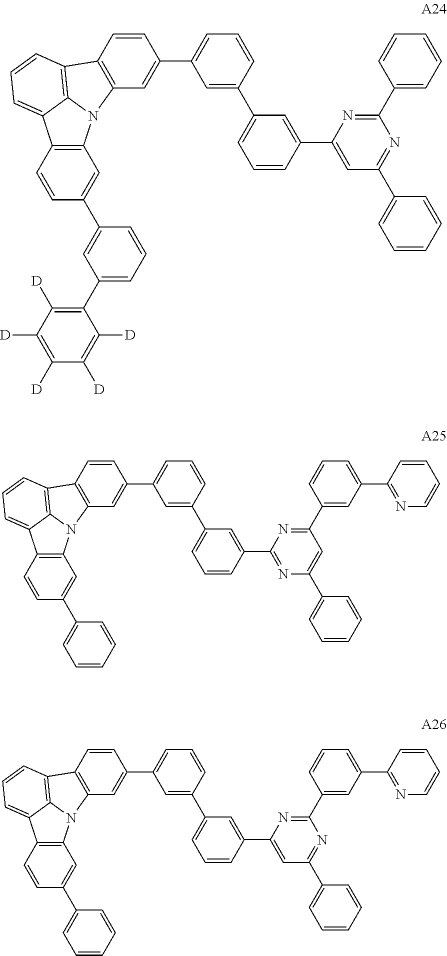 [SMILES: [2H]c1c([2H])c([2H])c(-c2cccc(-c3ccc4c5cccc6c7ccc(-c8cccc(-c9cccc(-c%10cc(-c%11ccccc%11)nc(-c%11ccccc%11)n%10)c9)c8)cc7n(c4c3)c65)c2)c([2H])c1[2H] ; c1ccc(-c2ccc3c4cccc5c6ccc(-c7cccc(-c8cccc(-c9nc(-c%10ccccc%10)cc(-c%10cccc(-c%11ccccn%11)c%10)n9)c8)c7)cc6n(c3c2)c45)cc1 ; c1ccc(-c2ccc3c4cccc5c6ccc(-c7cccc(-c8cccc(-c9cc(-c%10ccccc%10)nc(-c%10cccc(-c%11ccccn%11)c%10)n9)c8)c7)cc6n(c3c2)c45)cc1]

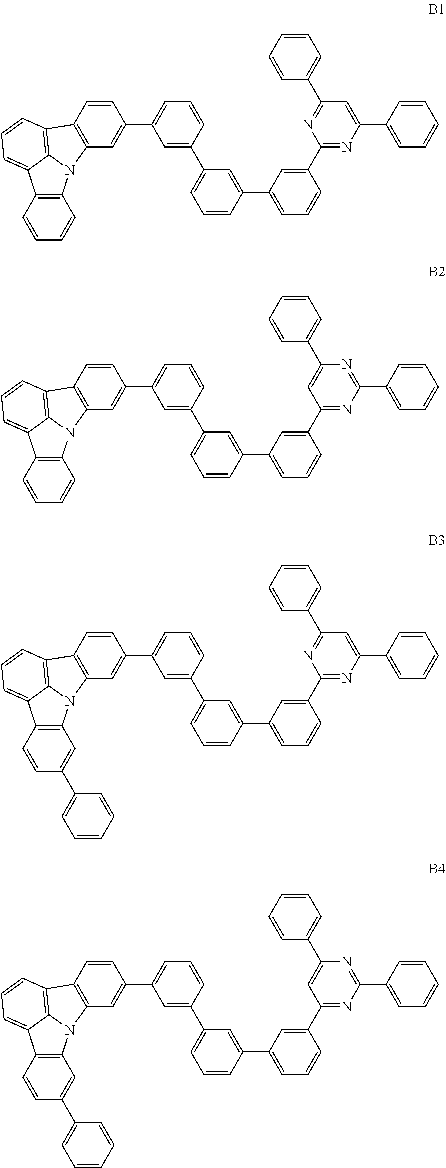 [SMILES: c1ccc(-c2cc(-c3ccccc3)nc(-c3cccc(-c4cccc(-c5cccc(-c6ccc7c8cccc9c%10ccccc%10n(c7c6)c98)c5)c4)c3)n2)cc1 ; c1ccc(-c2cc(-c3cccc(-c4cccc(-c5cccc(-c6ccc7c8cccc9c%10ccccc%10n(c7c6)c98)c5)c4)c3)nc(-c3ccccc3)n2)cc1 ; c1ccc(-c2ccc3c4cccc5c6ccc(-c7cccc(-c8cccc(-c9cccc(-c%10nc(-c%11ccccc%11)cc(-c%11ccccc%11)n%10)c9)c8)c7)cc6n(c3c2)c45)cc1 ; c1ccc(-c2ccc3c4cccc5c6ccc(-c7cccc(-c8cccc(-c9cccc(-c%10cc(-c%11ccccc%11)nc(-c%11ccccc%11)n%10)c9)c8)c7)cc6n(c3c2)c45)cc1]

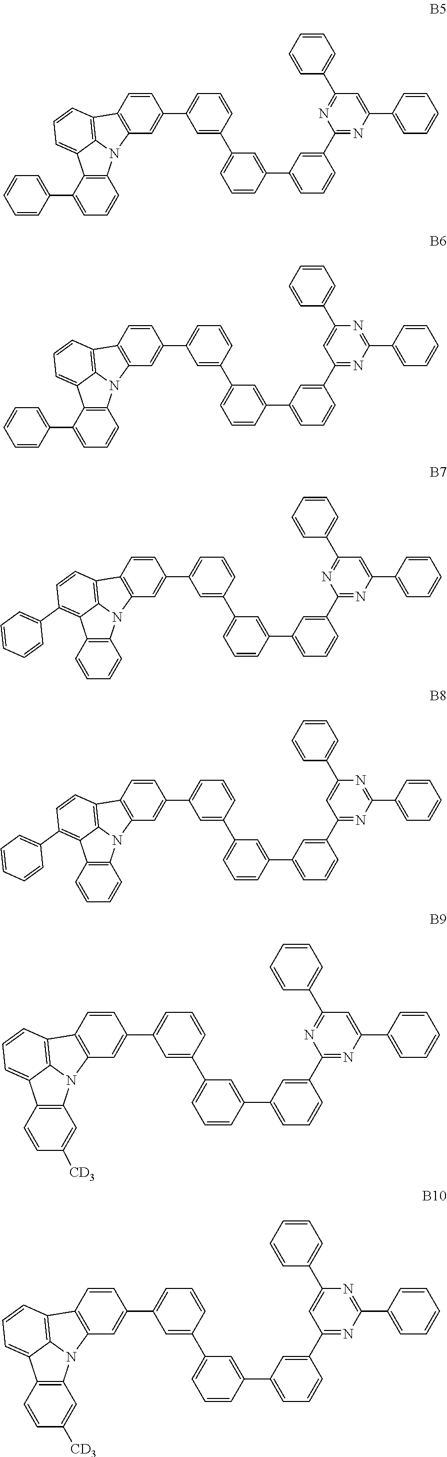

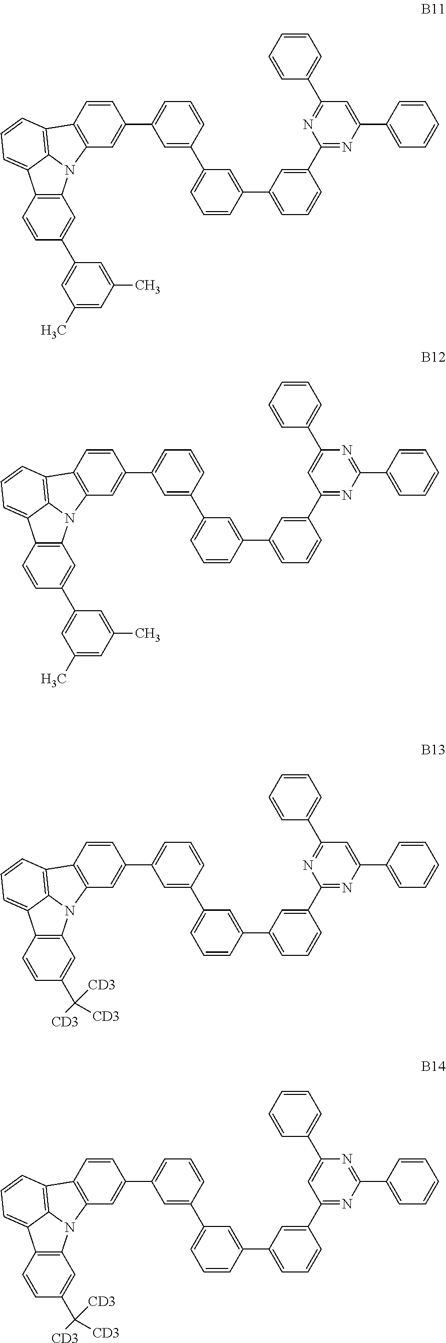

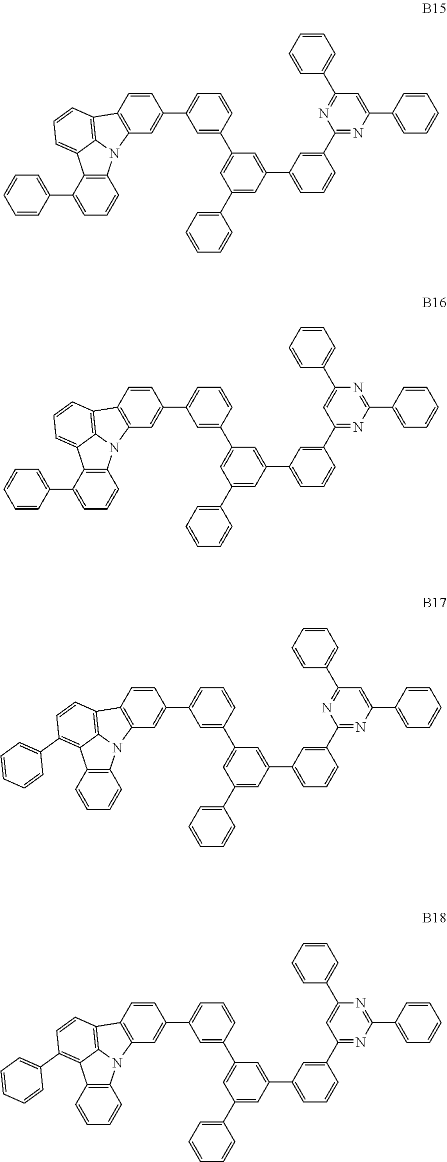 [SMILES: c1ccc(-c2cc(-c3cccc(-c4ccc5c6cccc7c8c(-c9ccccc9)cccc8n(c5c4)c67)c3)cc(-c3cccc(-c4nc(-c5ccccc5)cc(-c5ccccc5)n4)c3)c2)cc1 ; c1ccc(-c2cc(-c3cccc(-c4ccc5c6cccc7c8c(-c9ccccc9)cccc8n(c5c4)c67)c3)cc(-c3cccc(-c4cc(-c5ccccc5)nc(-c5ccccc5)n4)c3)c2)cc1 ; c1ccc(-c2cc(-c3cccc(-c4ccc5c6ccc(-c7ccccc7)c7c8ccccc8n(c5c4)c67)c3)cc(-c3cccc(-c4nc(-c5ccccc5)cc(-c5ccccc5)n4)c3)c2)cc1 ; c1ccc(-c2cc(-c3cccc(-c4ccc5c6ccc(-c7ccccc7)c7c8ccccc8n(c5c4)c67)c3)cc(-c3cccc(-c4cc(-c5ccccc5)nc(-c5ccccc5)n4)c3)c2)cc1]

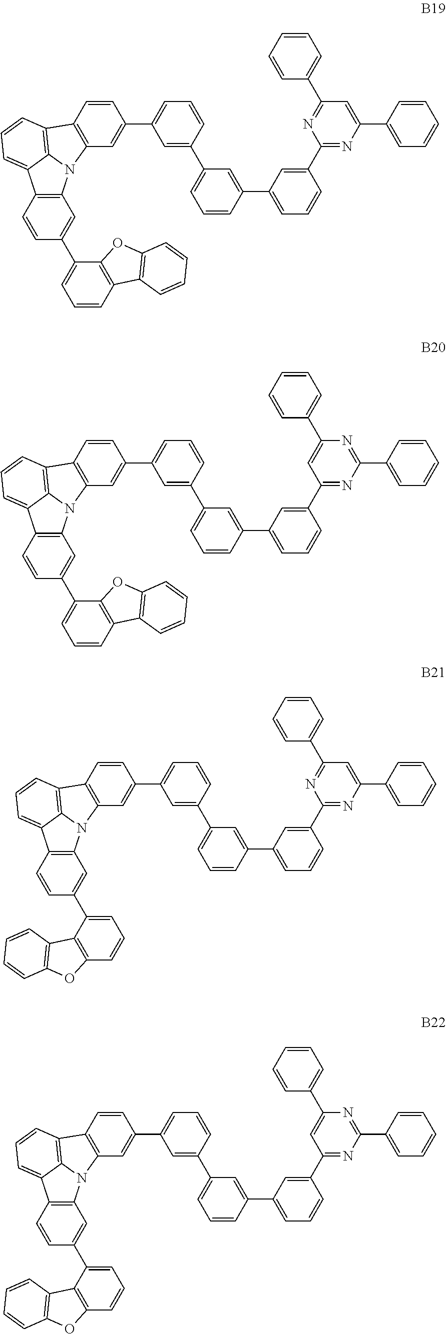

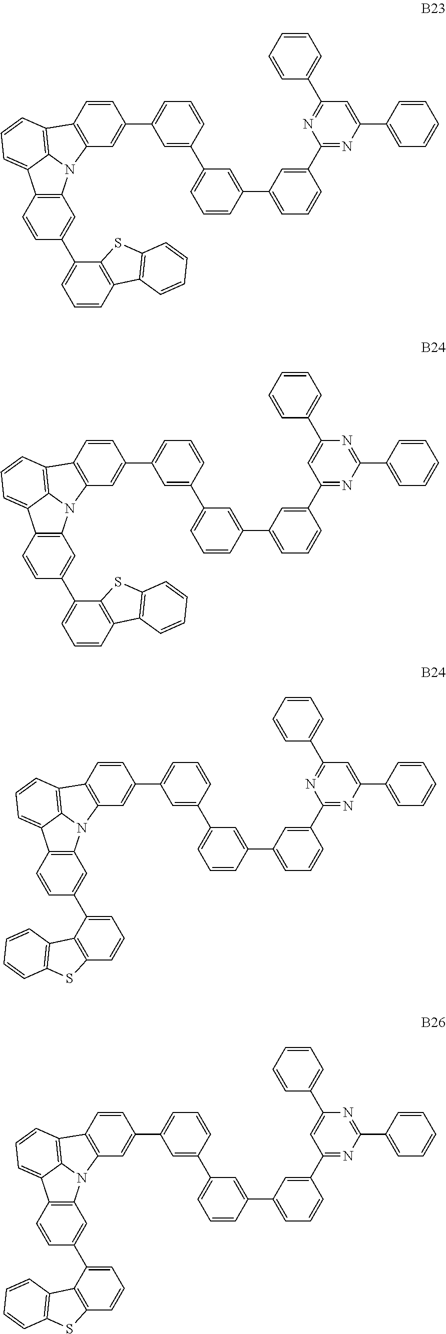

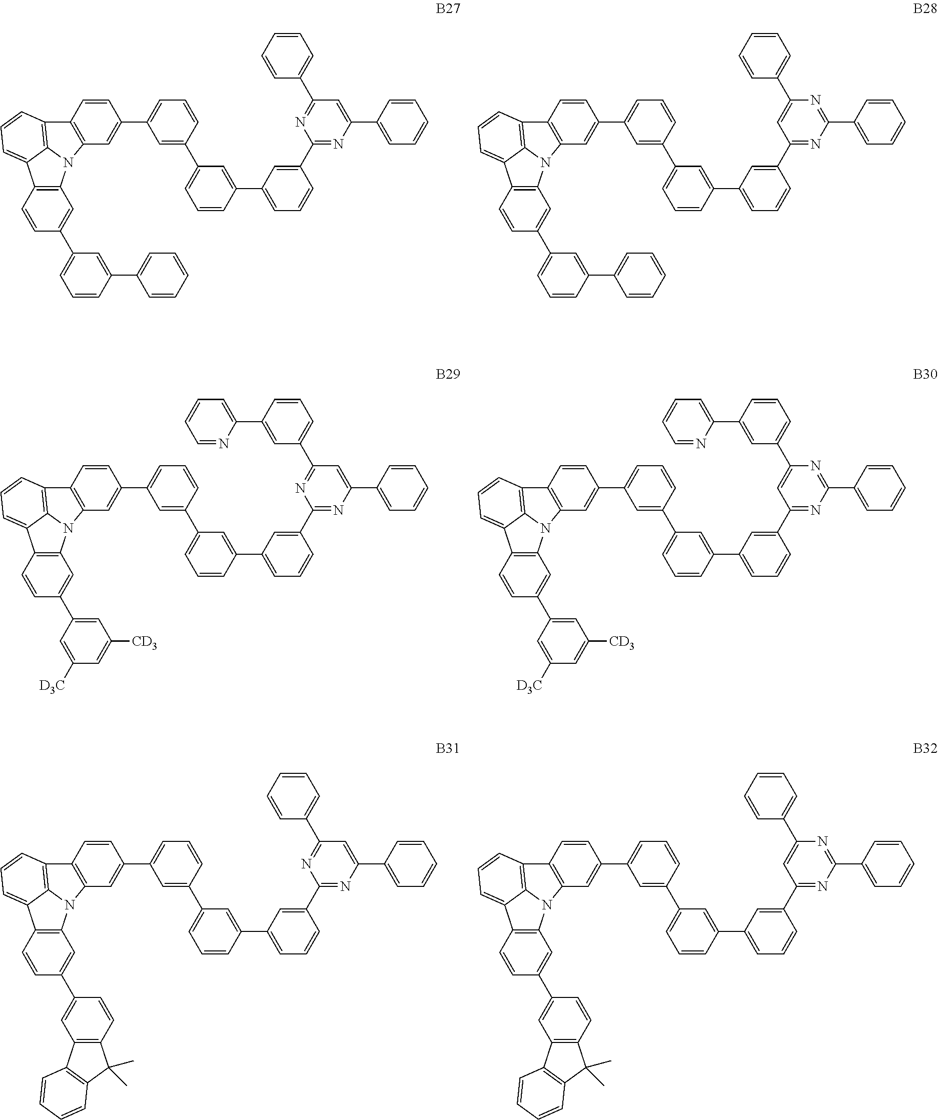

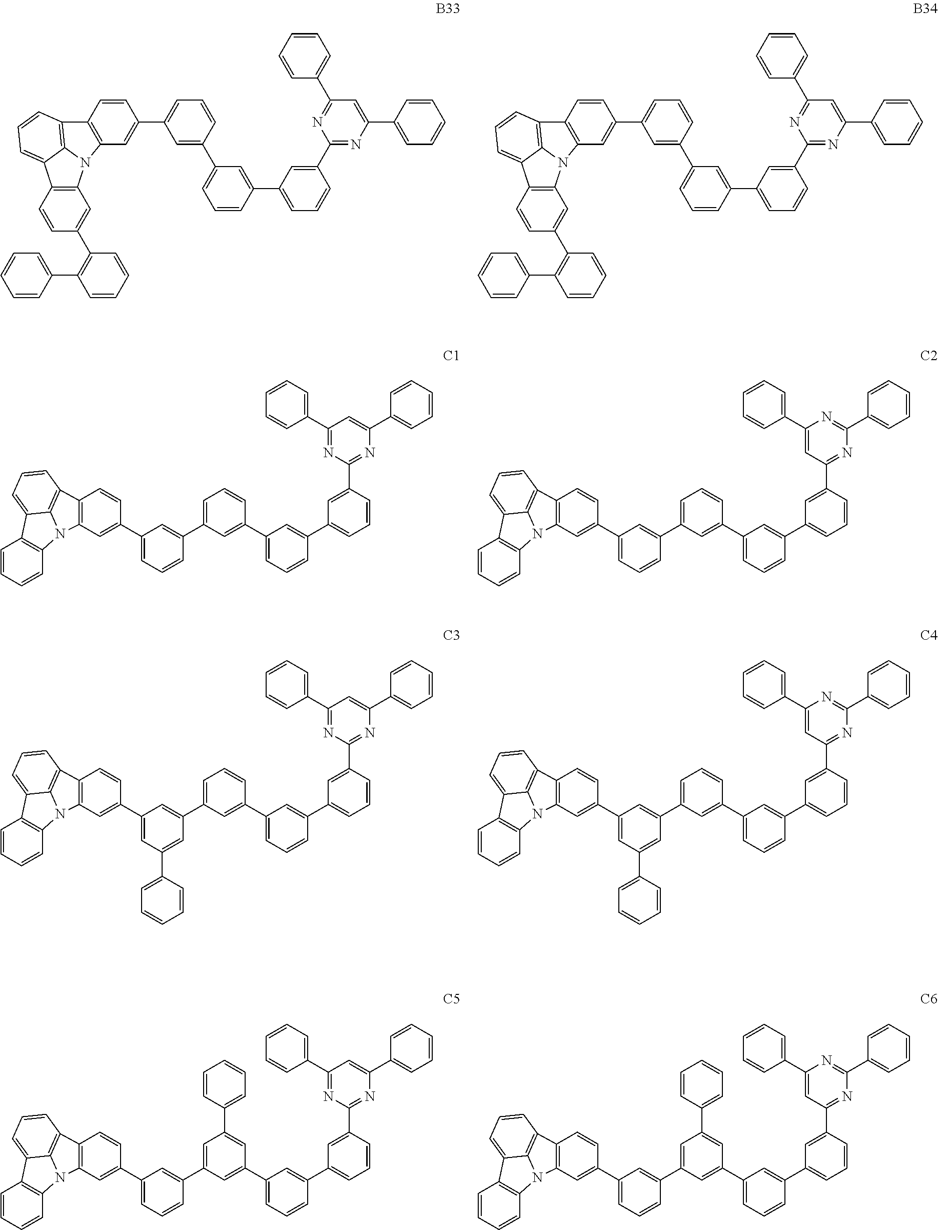

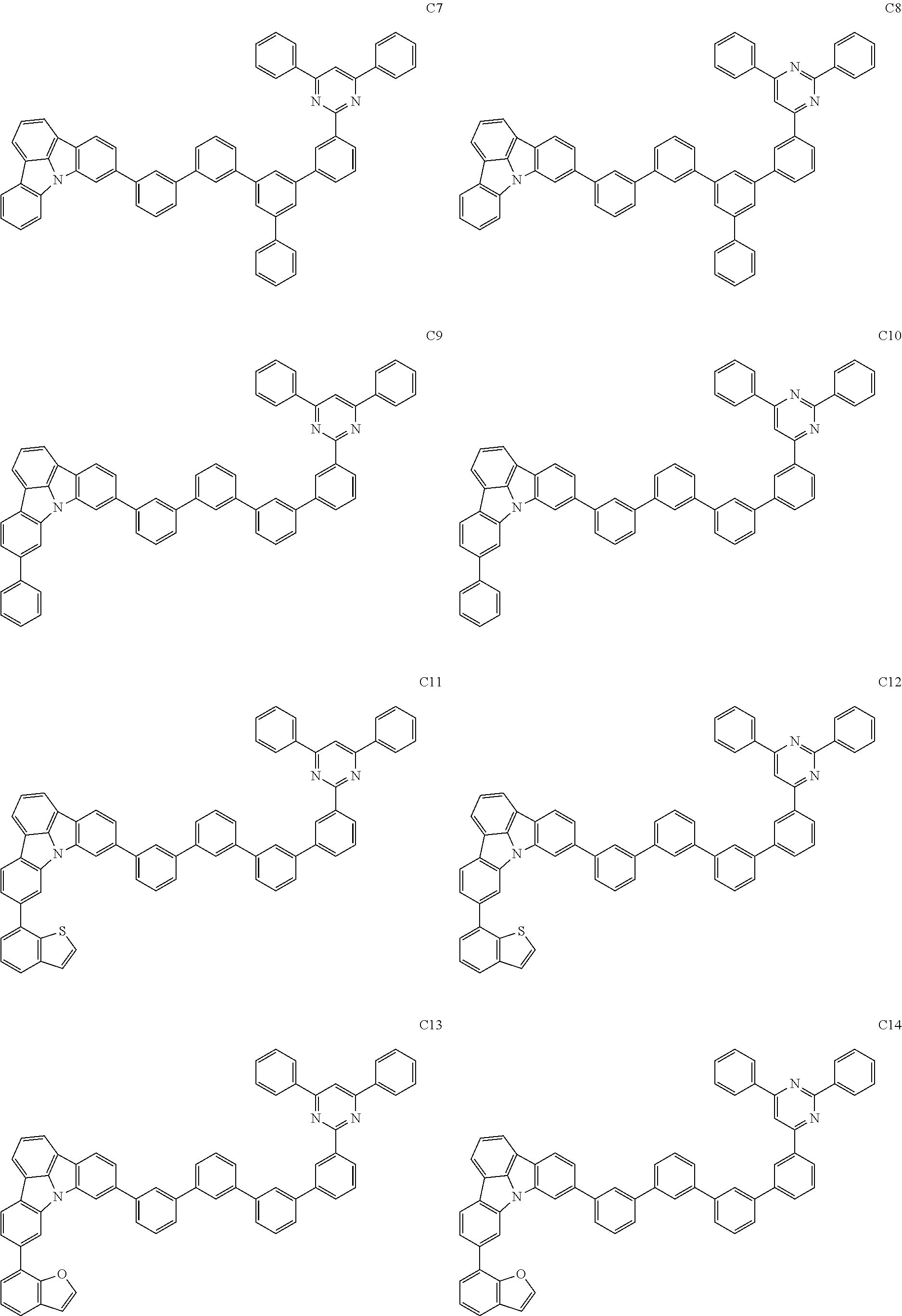

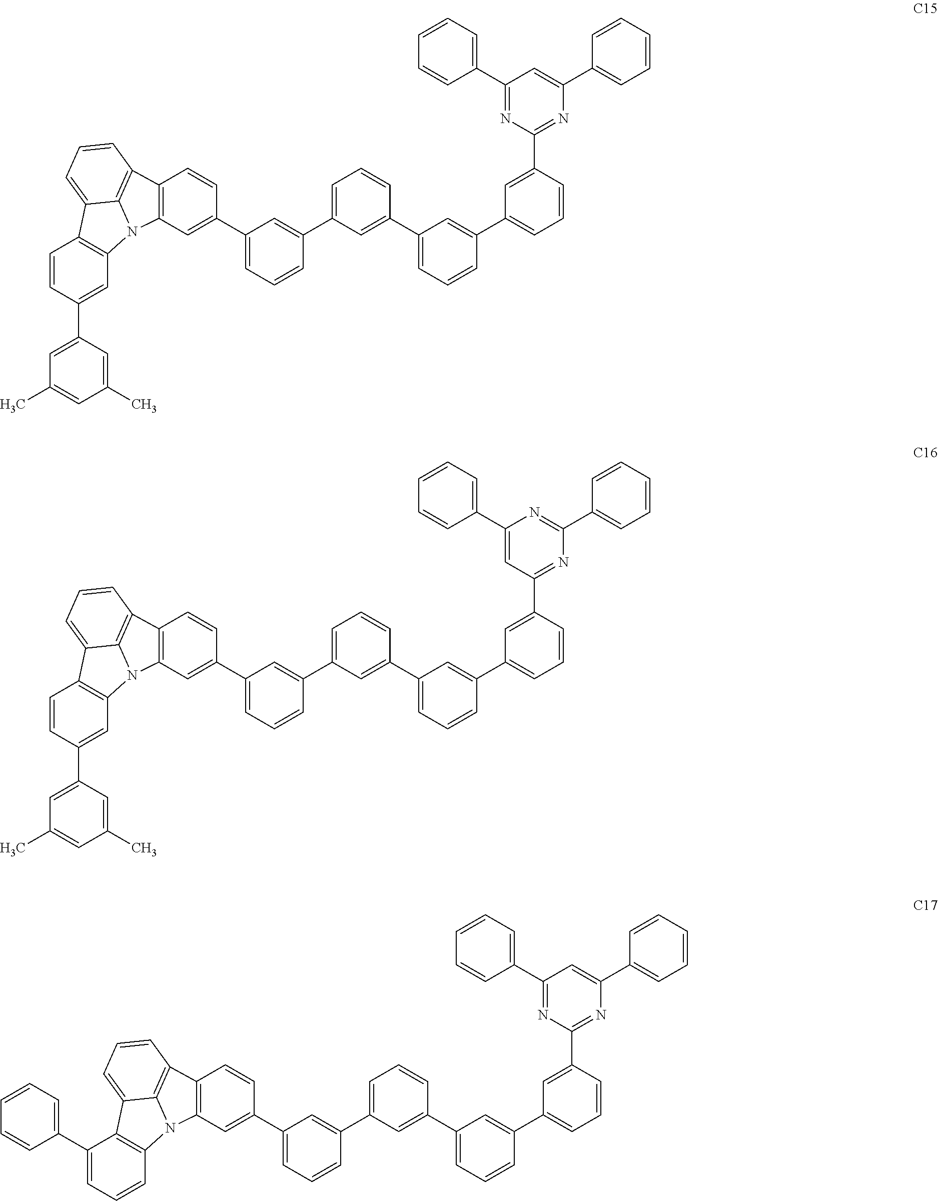 [SMILES: Cc1cc(C)cc(-c2ccc3c4cccc5c6ccc(-c7cccc(-c8cccc(-c9cccc(-c%10cccc(-c%11nc(-c%12ccccc%12)cc(-c%12ccccc%12)n%11)c%10)c9)c8)c7)cc6n(c3c2)c45)c1 ; Cc1cc(C)cc(-c2ccc3c4cccc5c6ccc(-c7cccc(-c8cccc(-c9cccc(-c%10cccc(-c%11cc(-c%12ccccc%12)nc(-c%12ccccc%12)n%11)c%10)c9)c8)c7)cc6n(c3c2)c45)c1 ; c1ccc(-c2cc(-c3ccccc3)nc(-c3cccc(-c4cccc(-c5cccc(-c6cccc(-c7ccc8c9cccc%10c%11c(-c%12ccccc%12)cccc%11n(c8c7)c9%10)c6)c5)c4)c3)n2)cc1]

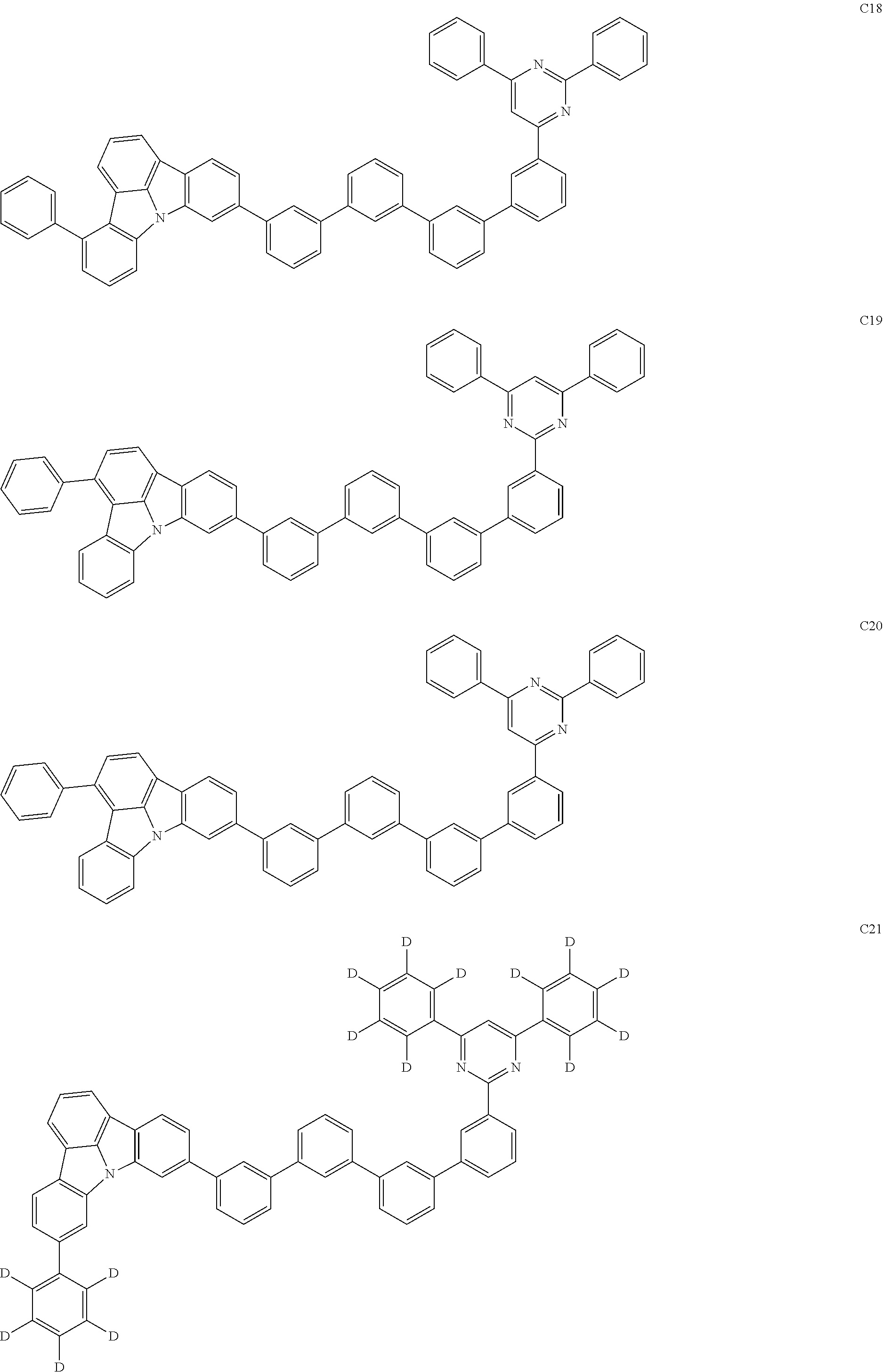

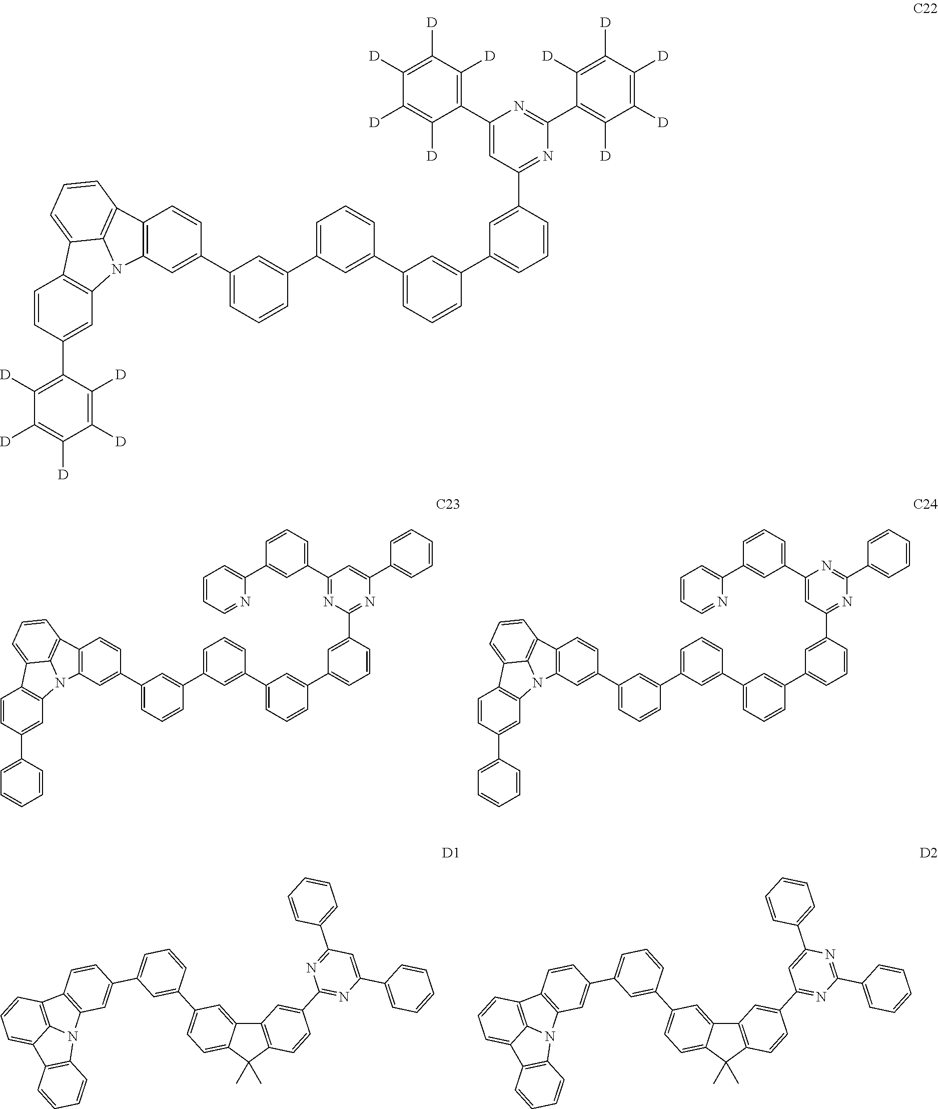

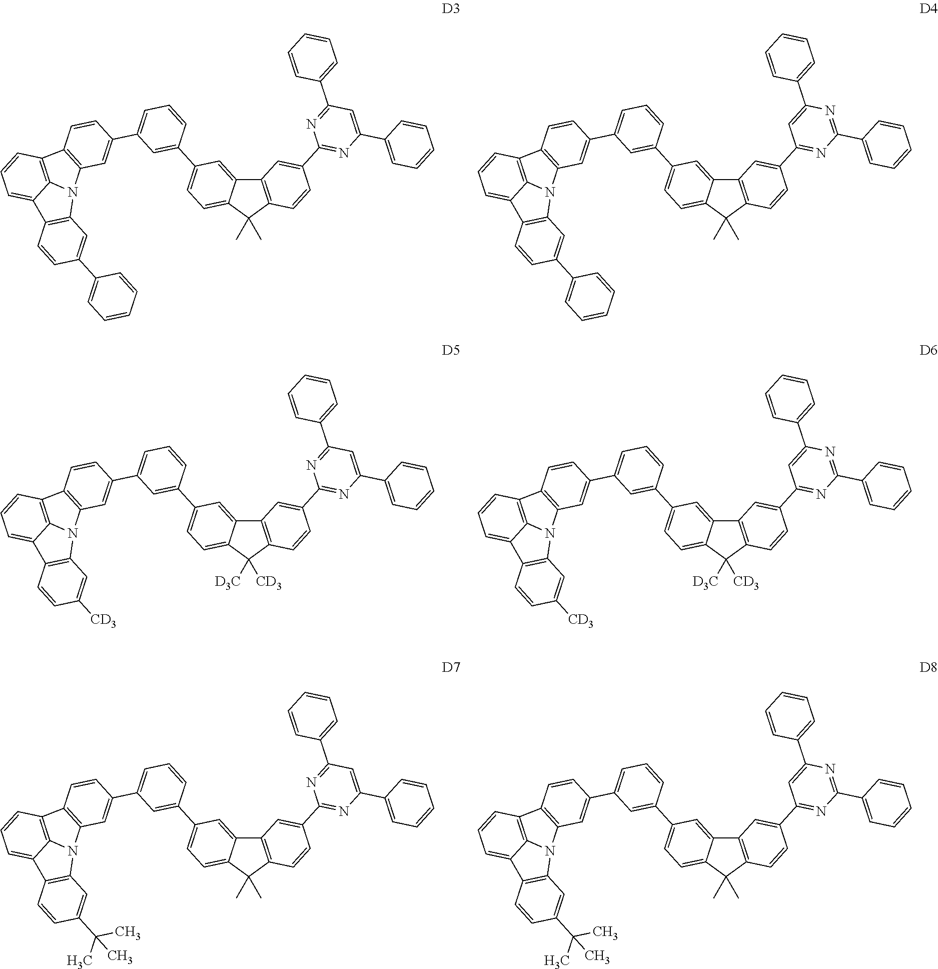

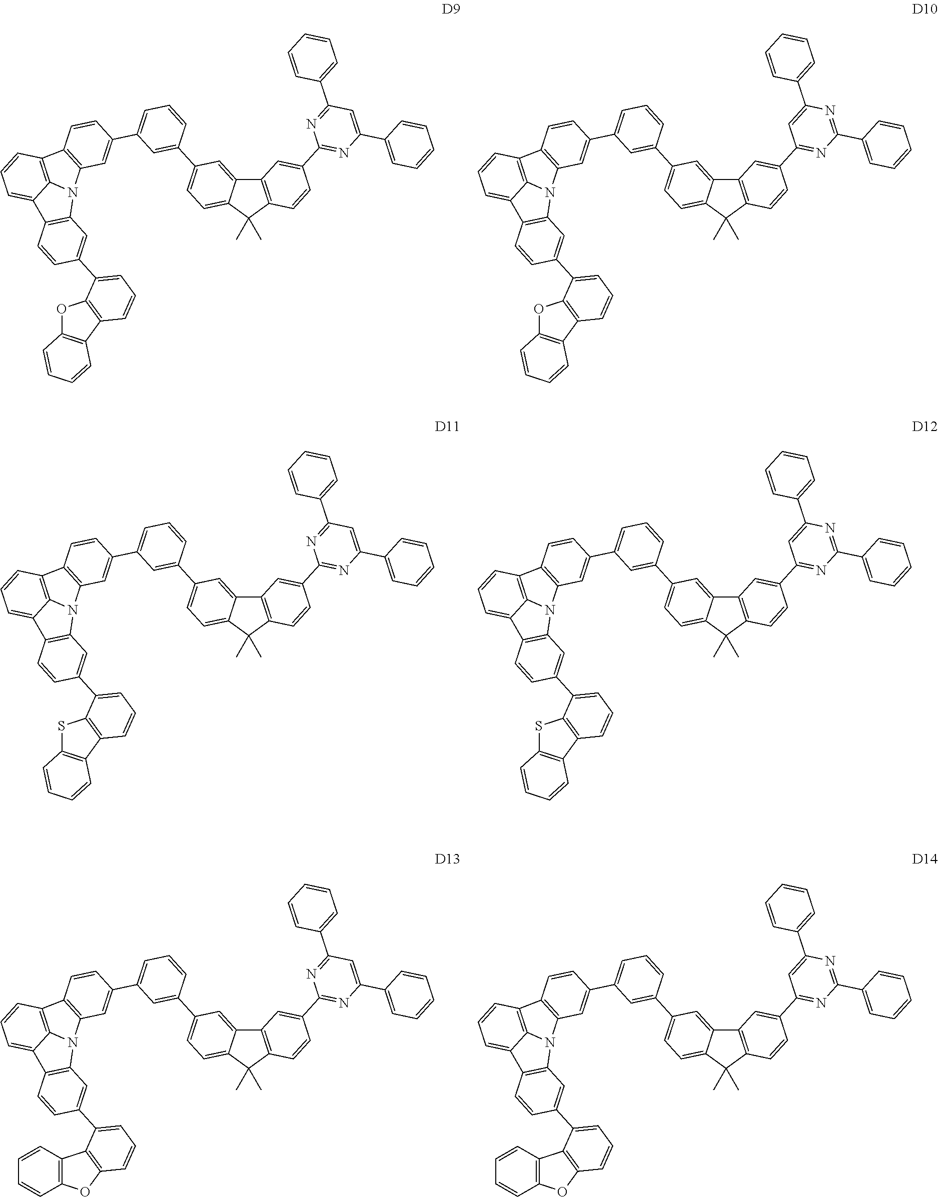

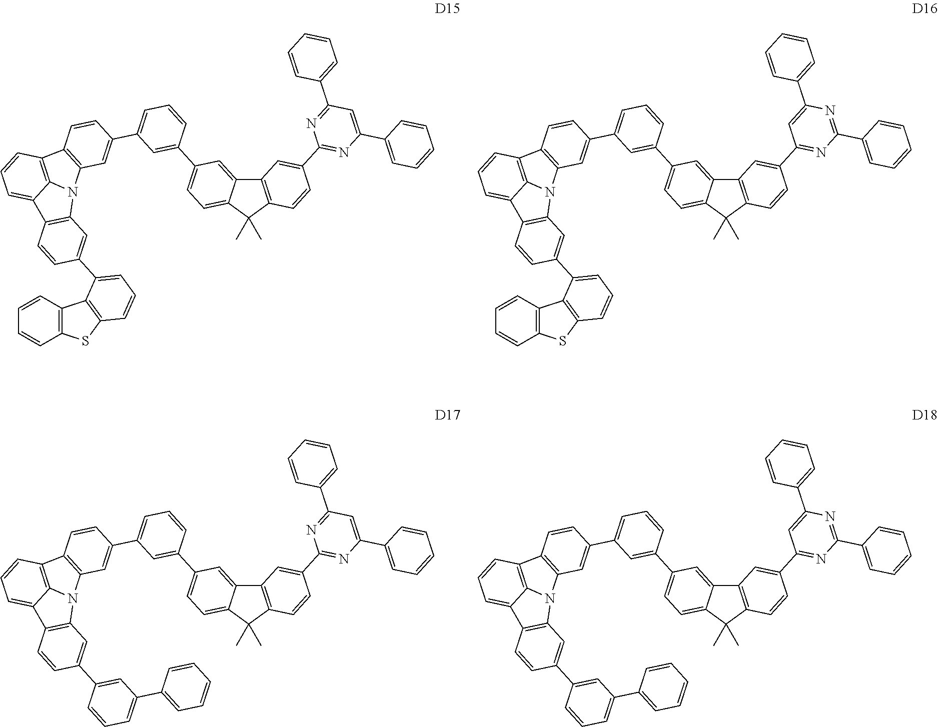

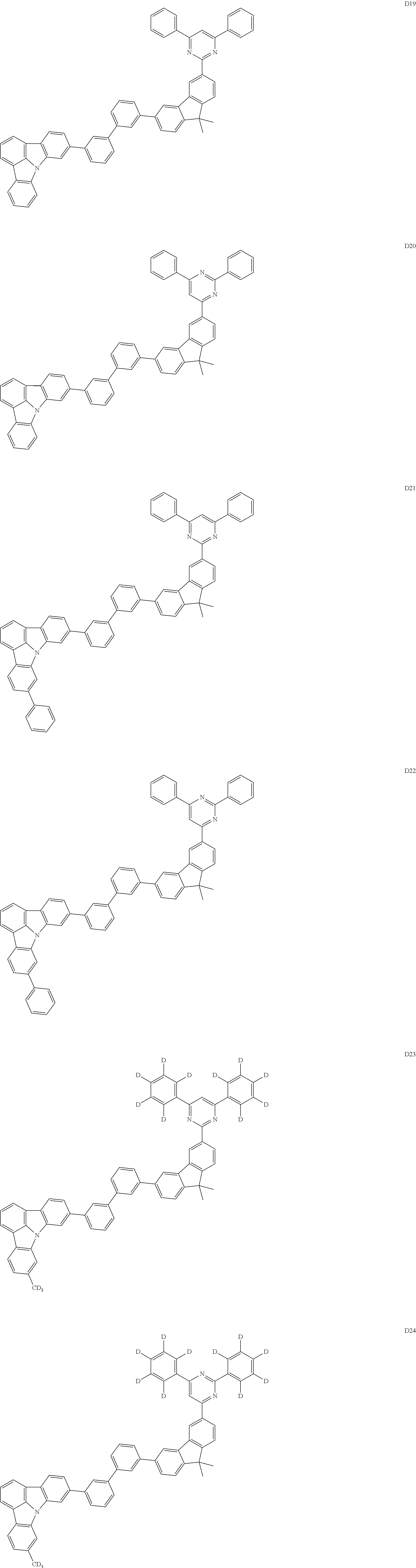

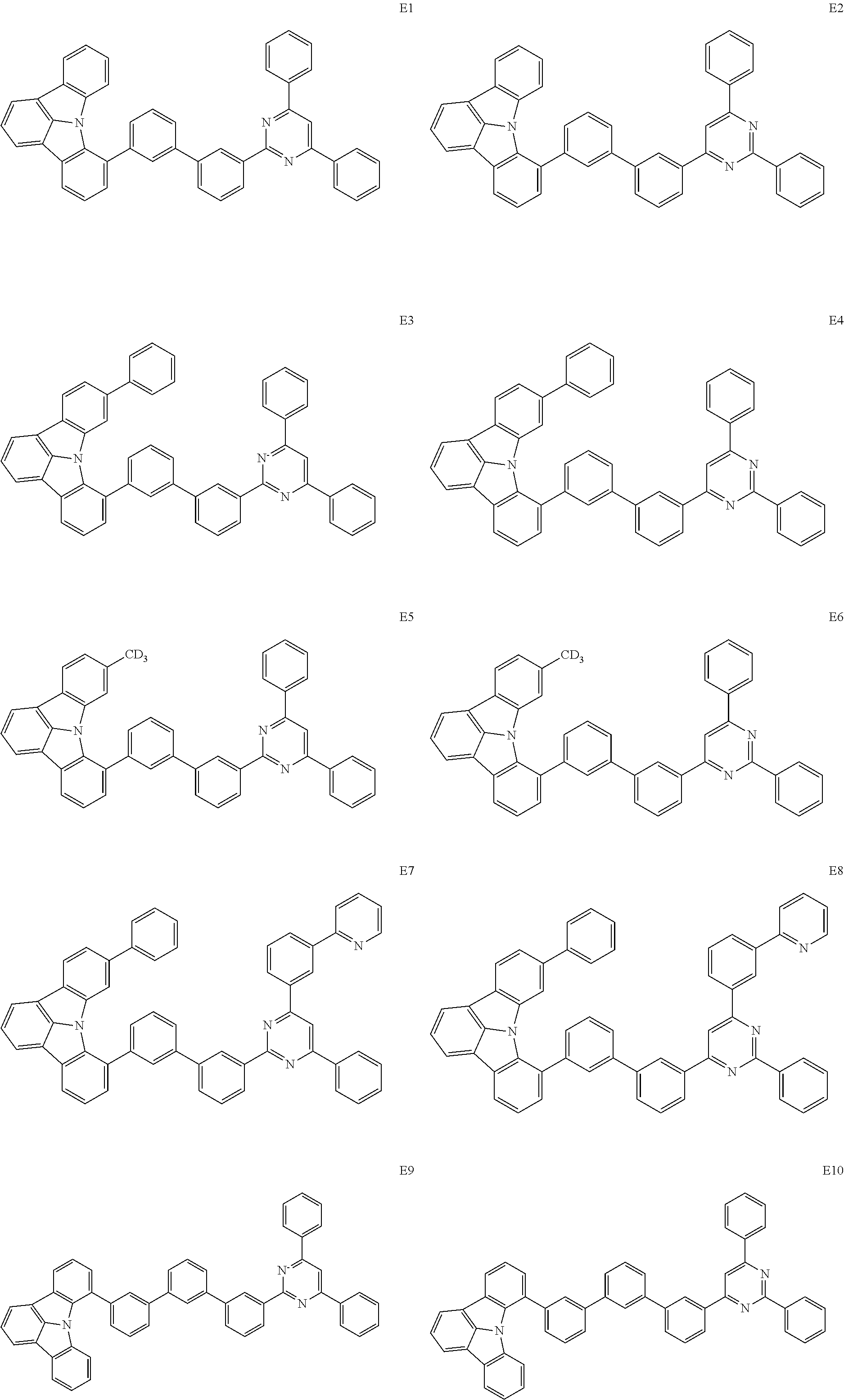

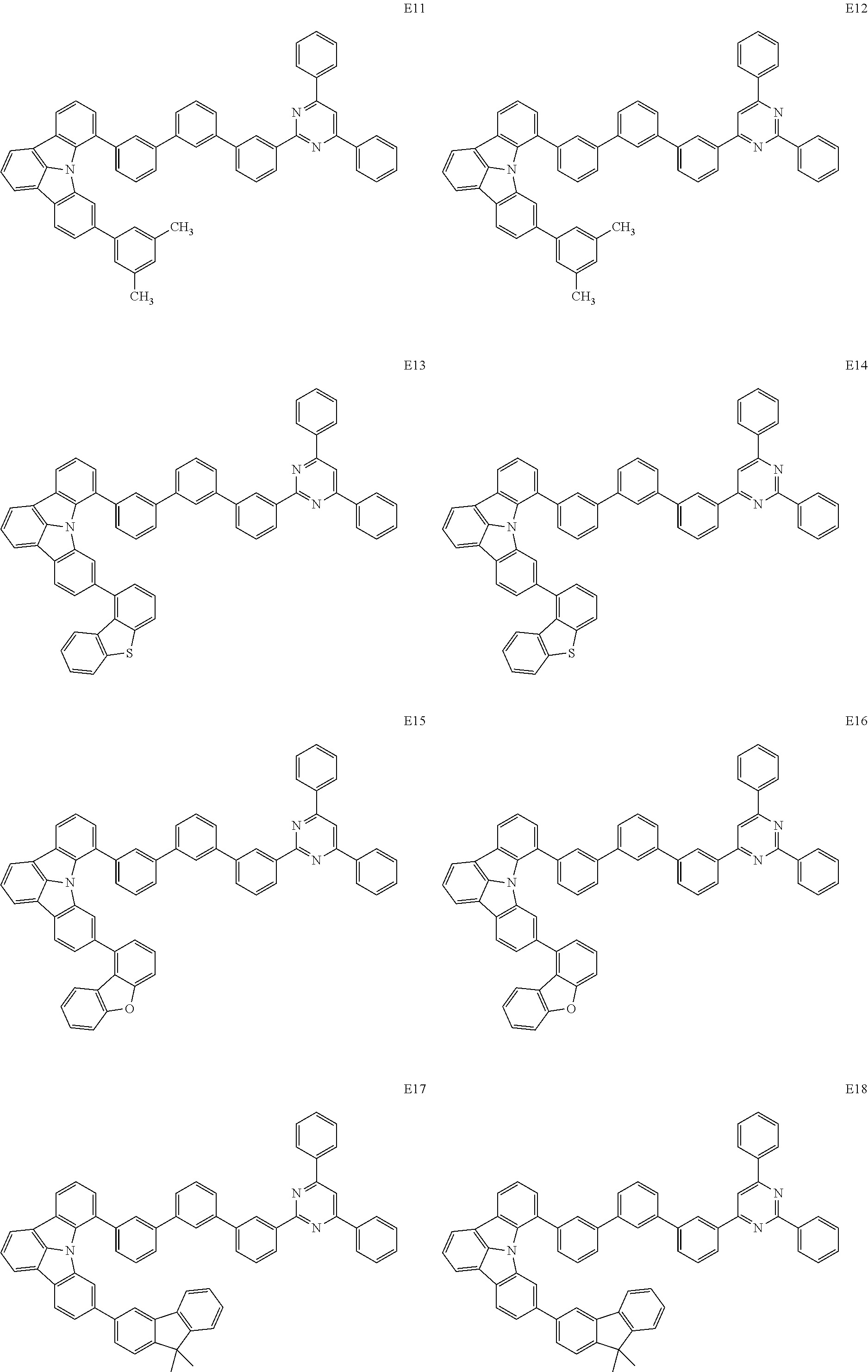

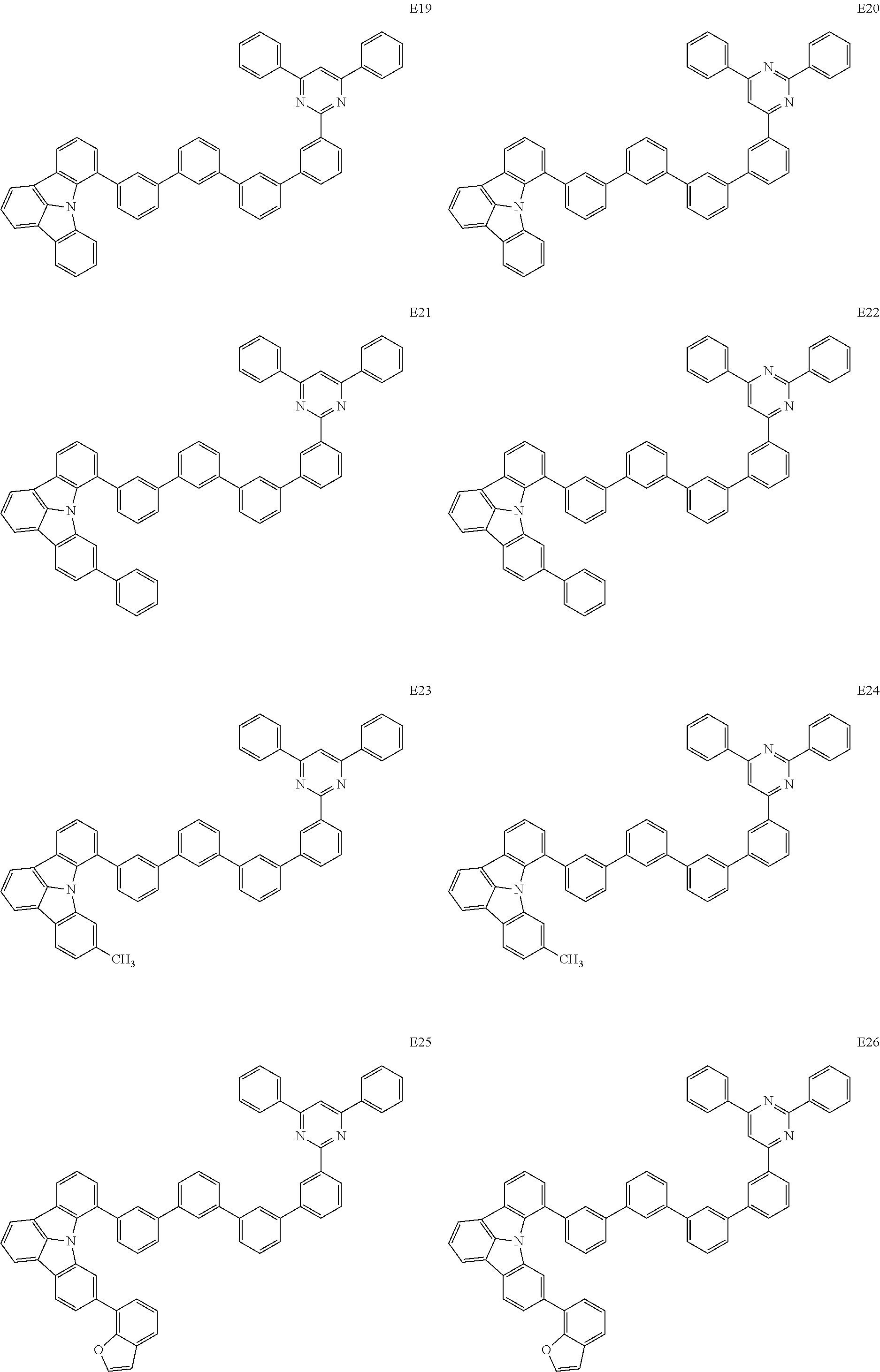

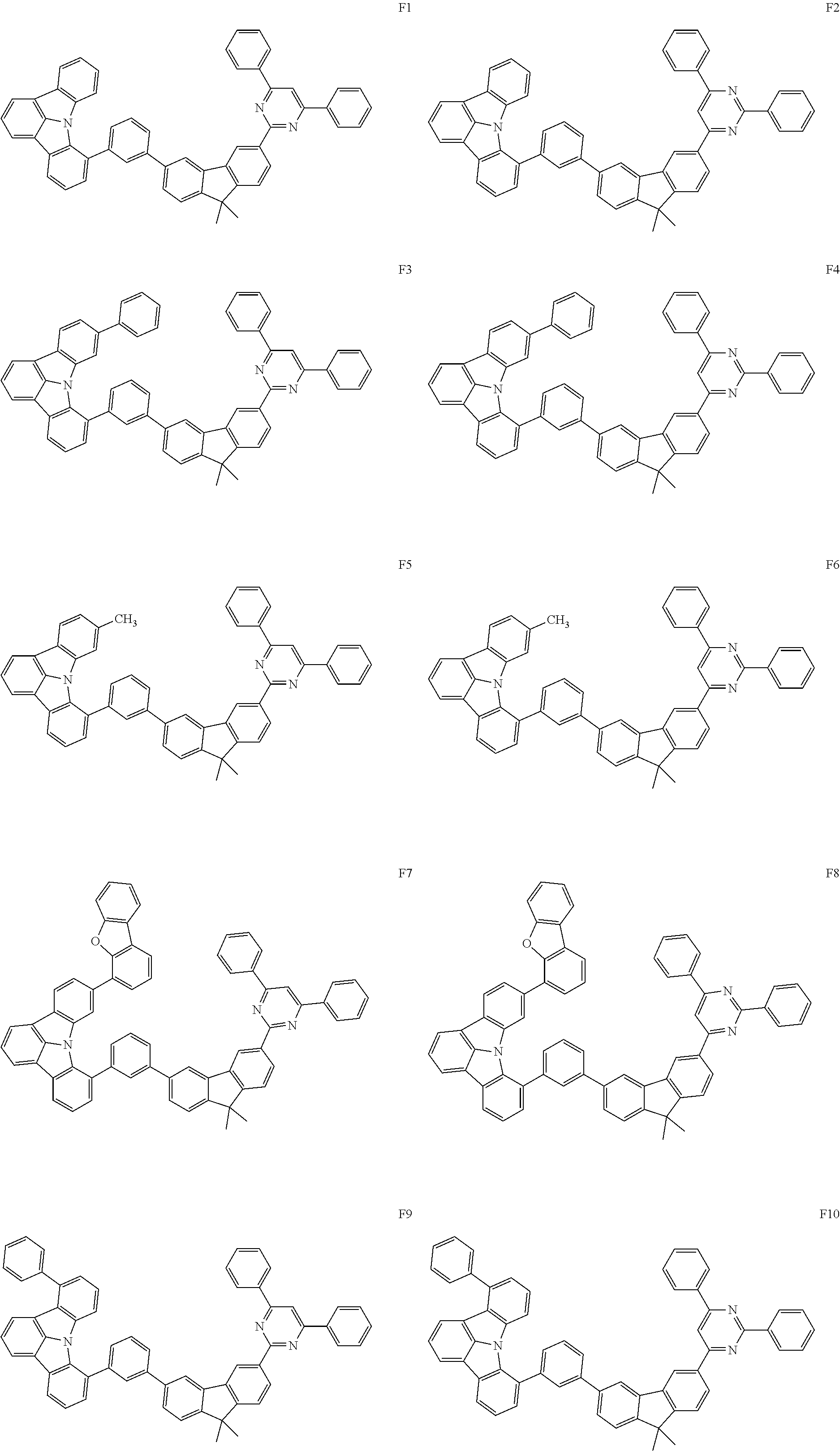

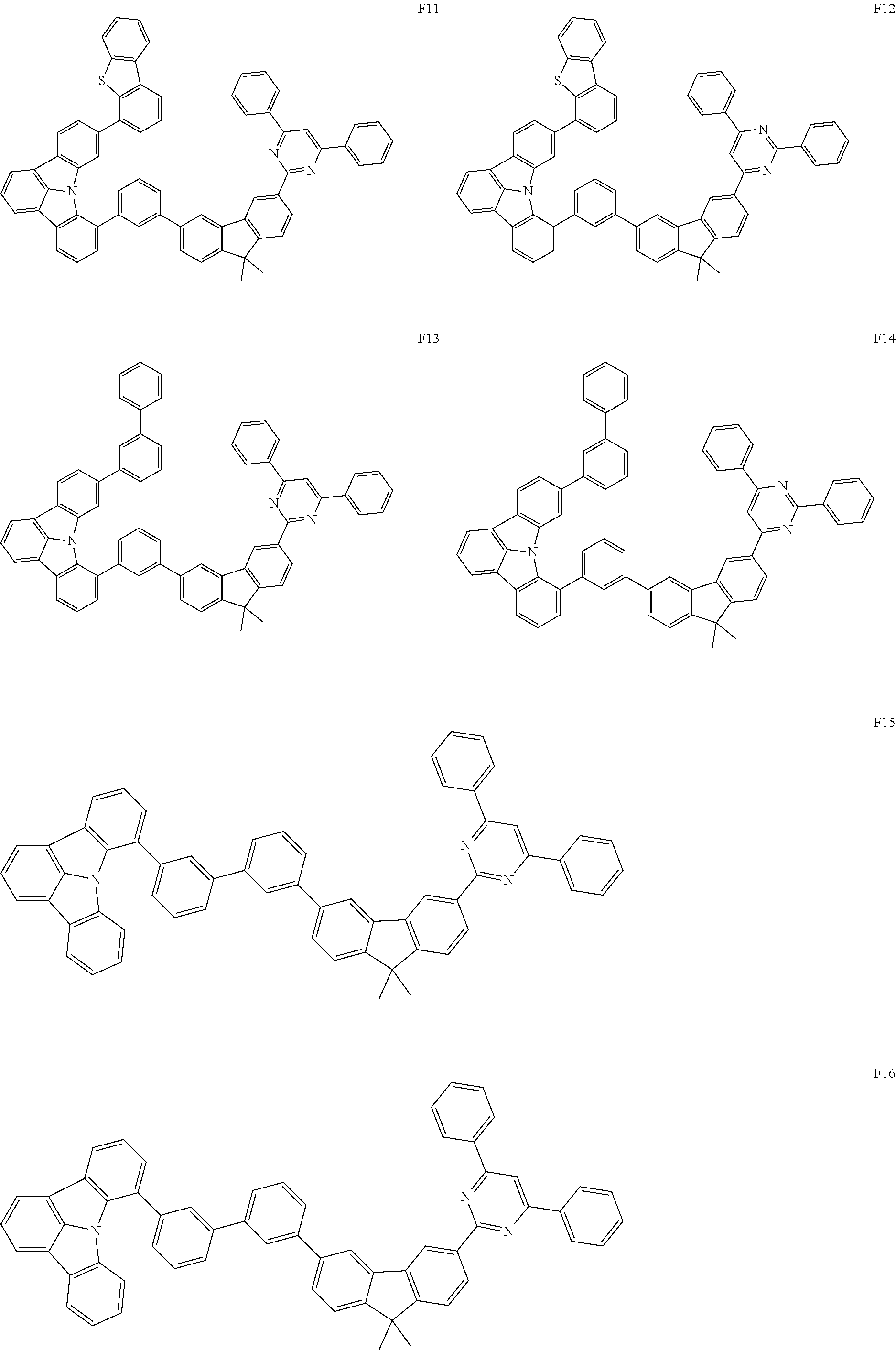

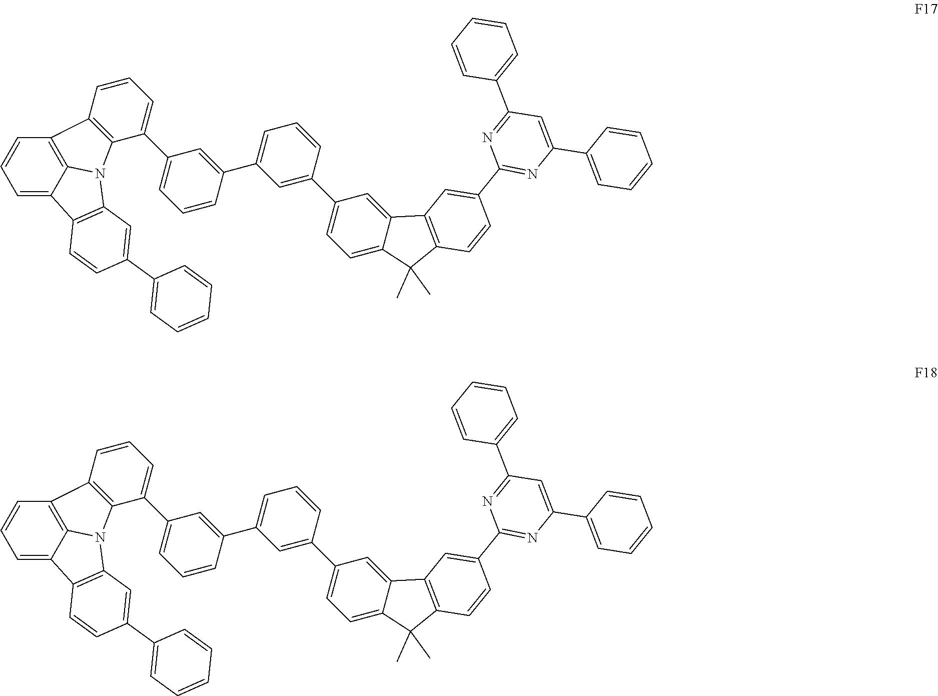 [SMILES: CC1(C)c2ccc(-c3cccc(-c4cccc(-c5cccc6c7cccc8c9ccc(-c%10ccccc%10)cc9n(c56)c87)c4)c3)cc2-c2cc(-c3nc(-c4ccccc4)cc(-c4ccccc4)n3)ccc21 ; CC1(C)c2ccc(-c3cccc(-c4cccc(-c5cccc6c7cccc8c9ccc(-c%10ccccc%10)cc9n(c56)c87)c4)c3)cc2-c2cc(-c3nc(-c4ccccc4)cc(-c4ccccc4)n3)ccc21]

The exemplary compounds belonging to the group A are compounds represented by the formula [1] wherein n=2 (compounds represented by the formula [3]). The compounds of the group A have a low molecular weight and a low sublimation temperature and therefore have an effect of increasing the margin of the sublimation temperature with respect to the decomposition temperature.

The exemplary compounds belonging to the group B are compounds represented by the formula [1] wherein n=3 (compounds represented by the formula [4]). The exemplary compounds have three phenylene groups bonded at the meta position as linking groups and therefore have relatively high thermal stability and T₁.

The exemplary compounds belonging to the group C are compounds represented by the formula [1] wherein n=4 (compounds represented by the formula [5]). The exemplary molecules have four phenylene groups bonded at the meta position as linking groups and therefore have a bent structure as a whole due to an increase in the number of rotational sites and have an increased intermolecular distance. This can avoid intermolecular stacking, resulting in high amorphousness.

The exemplary compounds belonging to the group D are compounds represented by the formula [6]. The exemplary compounds have a fluorenylene skeleton in addition to the phenylene groups as a linking group for bonding the indolocarbazole skeleton and the pyrimidine skeleton and therefore have a partially decreased number of rotational sites to make the molecule rigid and improved thermal stability.

The exemplary compounds belonging to the group E are compounds represented by the formula [2] wherein n=2 to 4. In the formula [2], the indolocarbazole skeleton is bonded to the phenylene groups of the linking group at the ortho position and is therefore bent with respect to the phenylene groups regardless of the number of phenylene groups, resulting in an increased intermolecular distance. This can avoid intermolecular stacking, resulting in high amorphousness.

The exemplary compounds belonging to the group F are compounds represented by the formula [7]. Similarly to the group E, the indolocarbazole skeleton is bent with respect to the phenylene groups. Furthermore, similarly to the group D, the number of rotational sites is partially decreased to make the molecule rigid and improve the thermal stability.

### (2) Organic Light-Emitting Device

Next, an organic light-emitting device according to the present embodiment is described below. The organic light-emitting device according to the present embodiment includes at least a first electrode, a second electrode, and an organic compound layer between these electrodes. One of the first electrode and the second electrode is a positive electrode, and the other is a negative electrode. In the organic light-emitting device according to the present embodiment, the organic compound layer may be a single layer or a laminate of a plurality of layers, provided that the organic compound layer has a light-emitting layer. When the organic compound layer is a laminate of a plurality of layers, the organic compound layer may have a hole injection layer, a hole transport layer, an electron-blocking layer, a hole/exciton-blocking layer, an electron transport layer, and/or an electron injection layer, in addition to a light-emitting layer. The light-emitting layer may be a single layer or a laminate of a plurality of layers.

In the organic light-emitting device according to the present embodiment, at least one layer of the organic compound layers contains the organic compound according to the present embodiment. More specifically, the organic compound according to the present embodiment is contained in one of the light-emitting layer, the hole injection layer, the hole transport layer, the electron-blocking layer, the hole/exciton-blocking layer, the electron transport layer, the electron injection layer, and the like. The organic compound according to the present embodiment can be contained in the light-emitting layer.

In the organic light-emitting device according to the present embodiment, when the organic compound according to the present embodiment is contained in the light-emitting layer, the light-emitting layer may be composed only of the organic compound according to the present embodiment or may be composed of the organic compound according to the present embodiment and another compound. The light-emitting layer can contain the organic compound according to the present embodiment and a phosphorescent compound or a hole transport compound. When the light-emitting layer is composed of the organic compound according to the present embodiment and another compound, the organic compound according to the present embodiment may be used as a host or a guest of the light-emitting layer. The compound may also be used as an assist material that may be contained in the light-emitting layer. The host is a compound with the highest mass ratio among the compounds constituting the light-emitting layer. The guest is a compound that has a lower mass ratio than the host among the compounds constituting the light-emitting layer and that is a principal light-emitting compound. The assist material is a compound that has a lower mass ratio than the host among the compounds constituting the light-emitting layer and that assists the guest in emitting light. The assist material is also referred to as a second host. The guest material may also be referred to as a first compound, and the assist material may also be referred to as a second compound. The T₁ of the second compound can be equal to or higher than the T₁ of the first compound.

When the organic compound according to the present embodiment is used as a host material in the light-emitting layer, the concentration of the host material is preferably 50% by mass or more and 99% by mass or less, more preferably 70% by mass or more and 99% by mass or less, of the entire light-emitting layer. Even when the host material is used in an amount of 99% by mass of the entire light-emitting layer, the compound according to the present embodiment is a material that is not easily crystallized and has high light emission efficiency and durability. This is due to the structural characteristics of the organic compound according to the present embodiment. Since all the phenylene groups constituting the linking group for bonding the indolocarbazole skeleton and the pyrimidine skeleton are bonded at the meta position, the entire molecule has a bent structure. This increases the intermolecular distance, and the compound is less likely to aggregate. It is possible to provide a light-emitting device that is less likely to have a crystal grain boundary associated with molecular aggregation even when an organic light-emitting device is driven and that has high light emission efficiency and long device lifetime.

The organic compound according to the present embodiment may be used as an assist material. The assist material has a role of complementing the carrier transport properties of the host material and promoting injection and transfer of carriers into the light-emitting layer. 30% by mass or more and 50% by mass or less of the organic compound can be used as an assist material. This can also improve the film properties of the light-emitting layer.

The present inventors have conducted various studies and have found that the organic compound according to the present embodiment can be used as a host material or an assist material in a light-emitting layer, particularly as a host material in the light-emitting layer, to provide a device that outputs bright light with high light emission efficiency and that has very high durability. The light-emitting layer may be monolayer or multilayer. For example, blue light emission may be chosen as an emission color of the present embodiment, and a light-emitting material of another emission color may be contained for color mixture. The term “multilayer”, as used herein, refers to a laminate of a light-emitting layer and another light-emitting layer. In such a case, the emission color of the organic light-emitting device is not limited to blue. More specifically, the emission color may be white or a neutral color. For white color emission, another light-emitting layer emits light of a color other than blue, such as red or green. Such a layer is formed by vapor deposition or coating. This is described in detail below in exemplary embodiments.

The organic compound according to the present embodiment can be used as a constituent material of an organic compound layer other than the light-emitting layer constituting the organic light-emitting device according to the present embodiment. More specifically, the organic compound according to the present embodiment may be used as a constituent material of an electron transport layer, an electron injection layer, a hole transport layer, a hole injection layer, a hole-blocking layer, and the like. In such a case, the emission color of the organic light-emitting device is not limited to blue. More specifically, the emission color may be white or a neutral color.

### <Other Compounds>

If necessary, the organic compound according to the present embodiment may be used in combination with a known low-molecular-weight or high-molecular-weight hole injection compound or hole transport compound, host compound, light-emitting compound, electron injection compound, electron transport compound, or the like. Examples of these compounds are described below.

The hole injection/transport material can be a material with high hole mobility to facilitate the injection of a hole from a positive electrode and to transport the injected hole to a light-emitting layer. Furthermore, a material with a high glass transition temperature can be used to reduce degradation of film quality, such as crystallization, in an organic light-emitting device. A low-molecular-weight or high-molecular-weight material with hole injection/transport ability is, for example, a triarylamine derivative, an aryl carbazole derivative, a phenylenediamine derivative, a stilbene derivative, a phthalocyanine derivative, a porphyrin derivative, polyvinylcarbazole, polythiophene, another electrically conductive polymer, or the like Furthermore, the hole injection/transport material can also be suitable for use in an electron-blocking layer. Specific examples of a compound that can be used as a hole injection/transport material include, but are not limited to, the following.

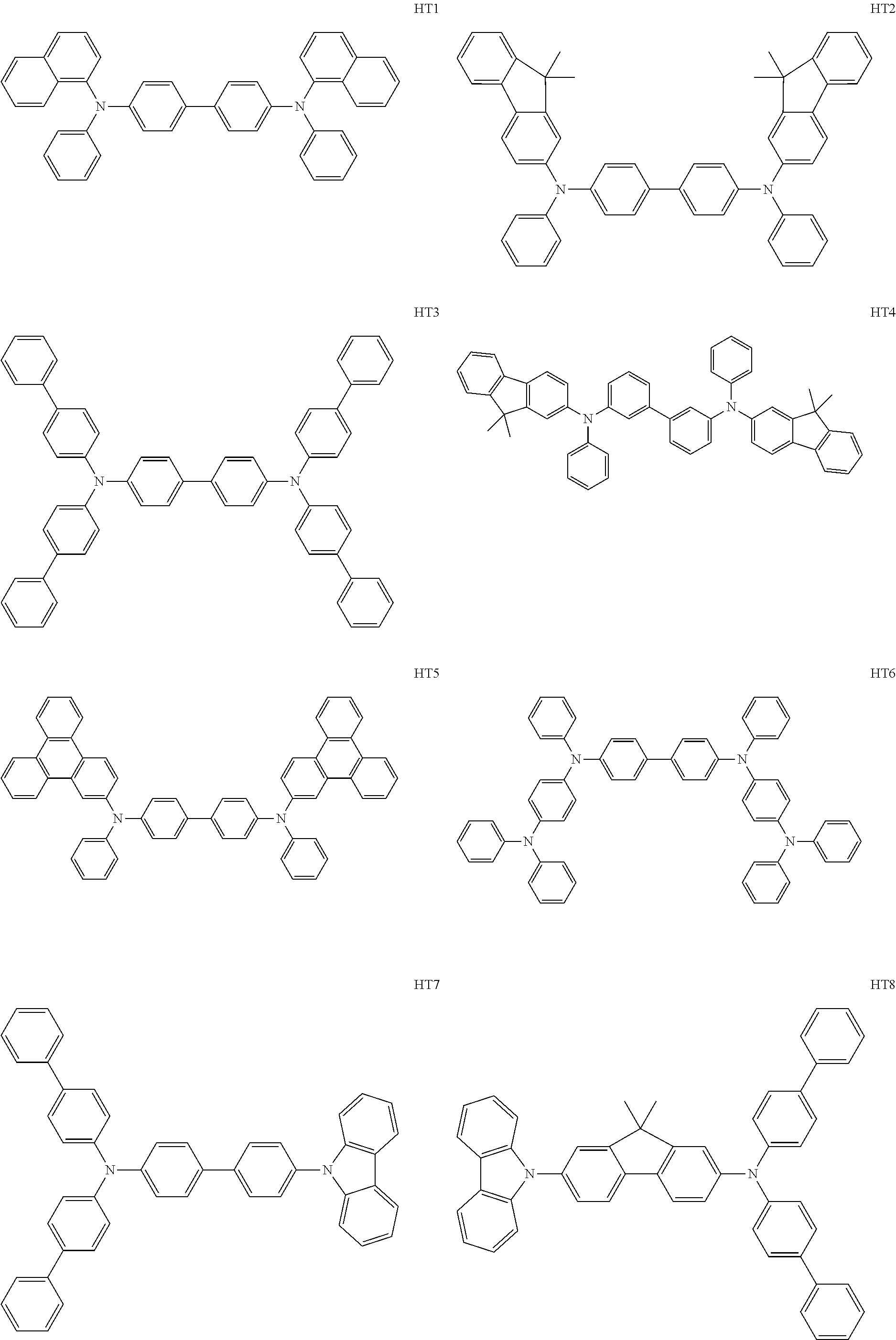

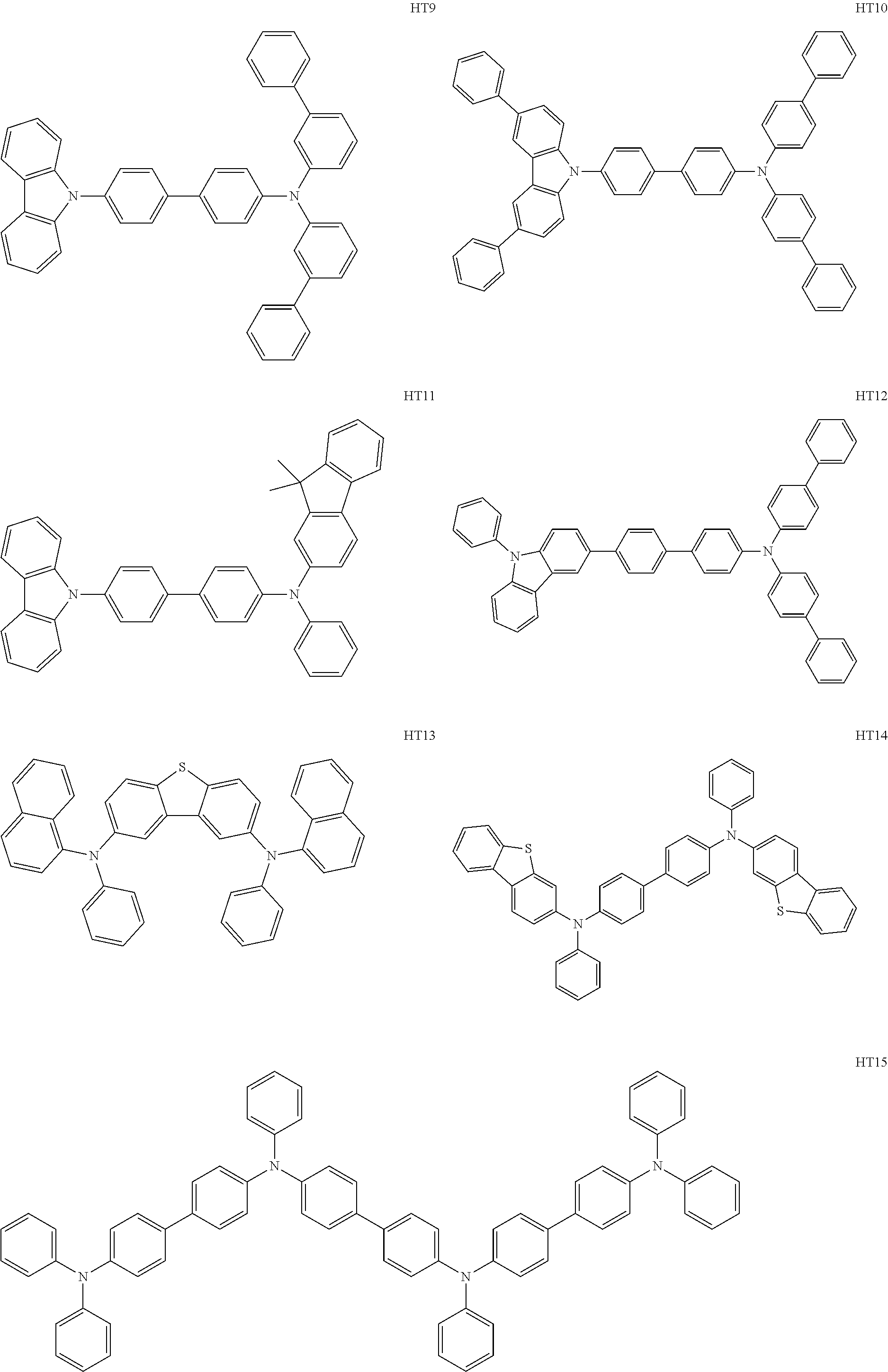

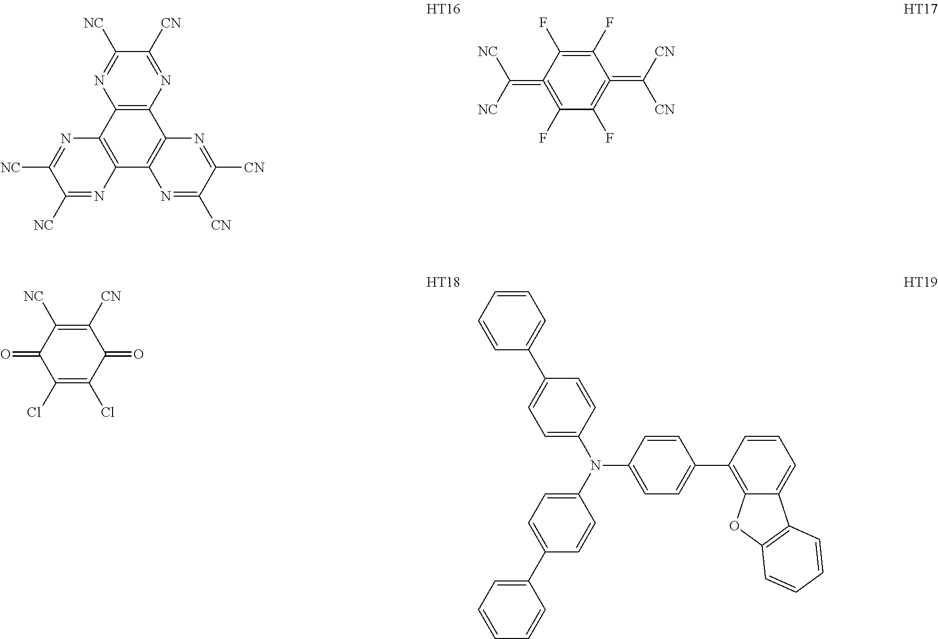

Among these hole transport materials, HT16 to HT18 can be used for a layer in contact with the positive electrode to decrease drive voltage. HT16 is widely used for organic light-emitting devices. HT2, HT3, HT4, HT5, HT6, HT10, HT12, and HT15 may be used for an organic compound layer adjacent to HT16. Furthermore, a plurality of materials may be used for one organic compound layer.

A light-emitting material mainly related to the light-emitting function is, for example, a fused-ring compound (for example, a fluorene derivative, a naphthalene derivative, a pyrene derivative, a perylene derivative, a tetracene derivative, an anthracene derivative, rubrene, or the like), a quinacridone derivative, a coumarin derivative, a stilbene derivative, an organoaluminum complex, such as tris(8-quinolinolato)aluminum, an iridium complex, a platinum complex, a rhenium complex, a copper complex, an europium complex, a ruthenium complex, a polymer derivative, such as a poly(phenylene vinylene) derivative, a polyfluorene derivative, or a polyphenylene derivative, or the like. Specific examples of a compound that can be used as a light-emitting material include, but are not limited to, the following.

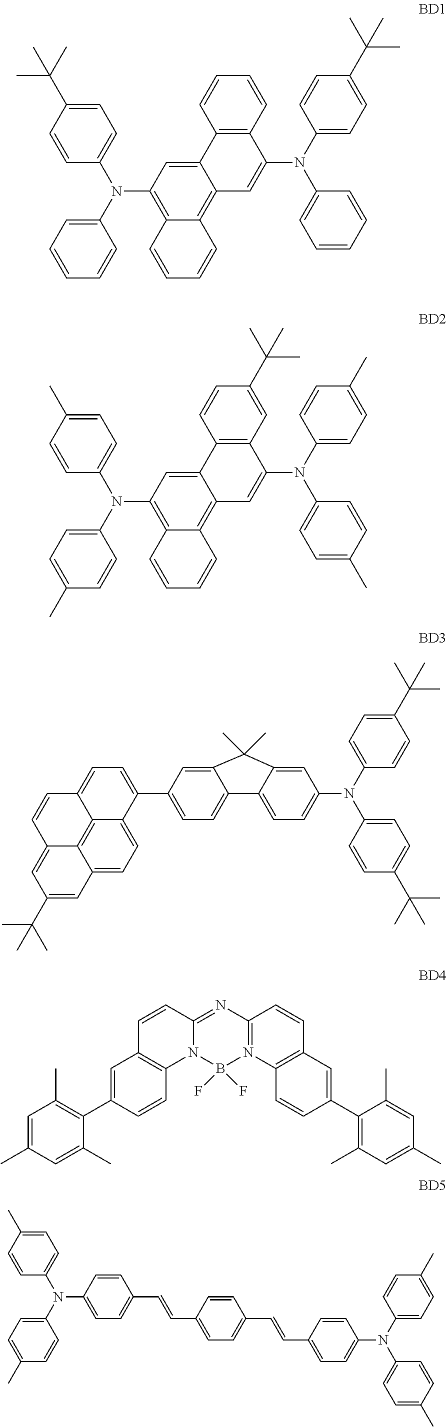

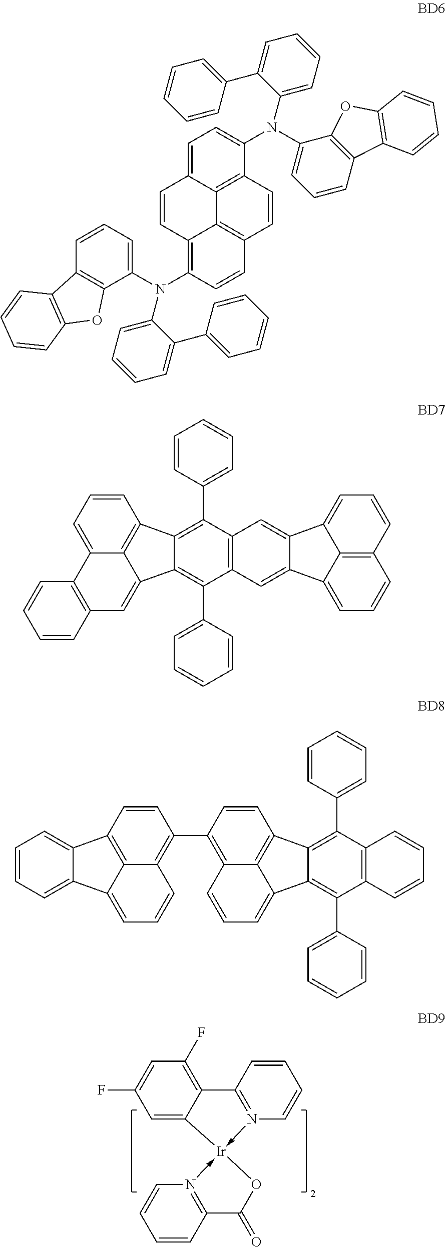

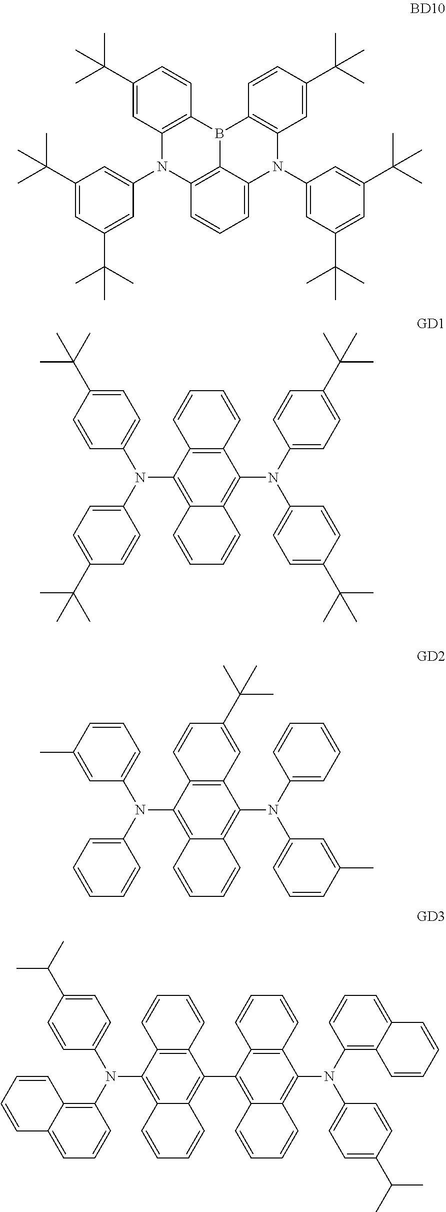

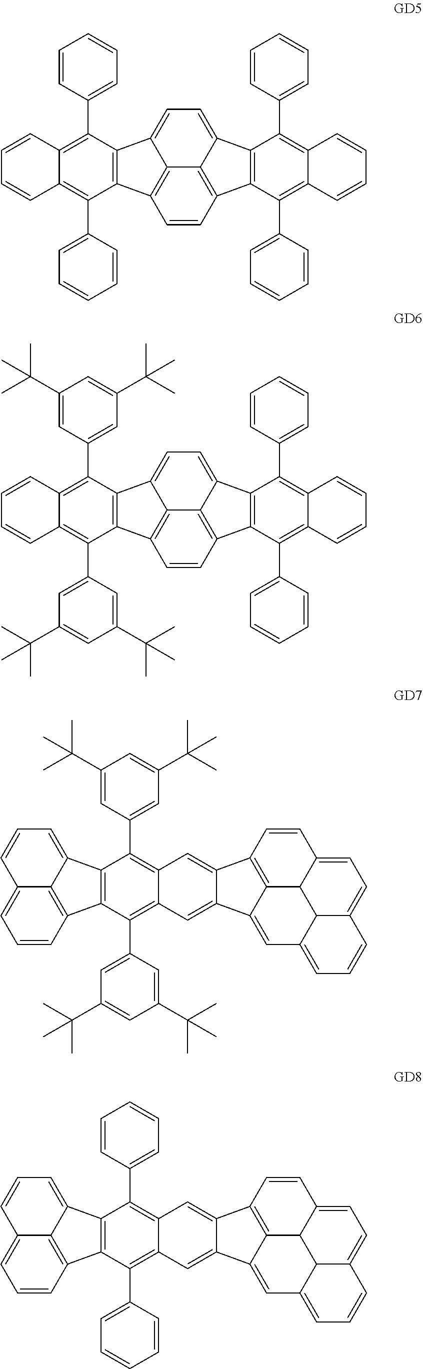

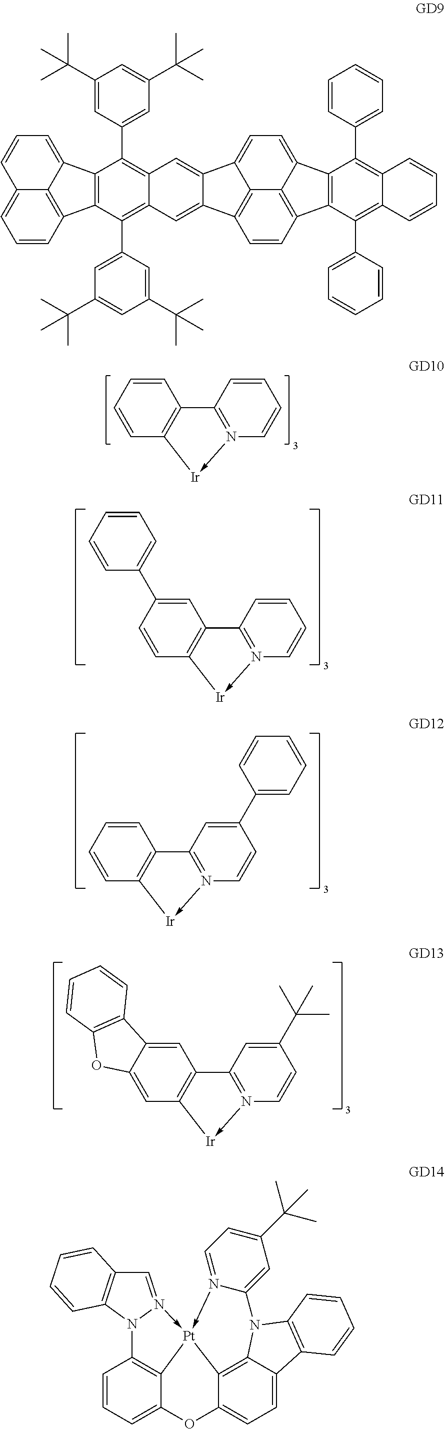

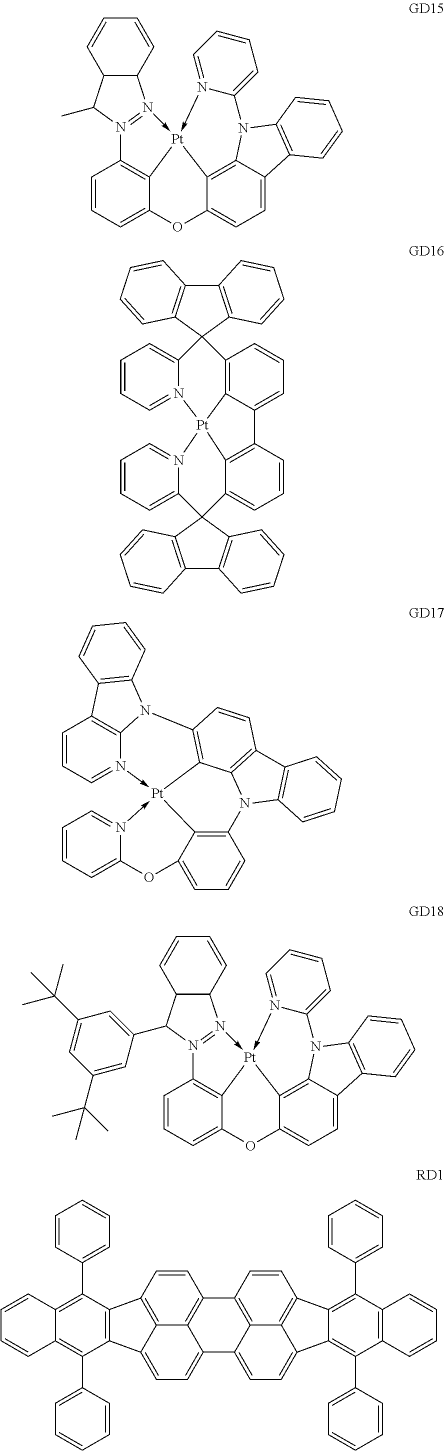

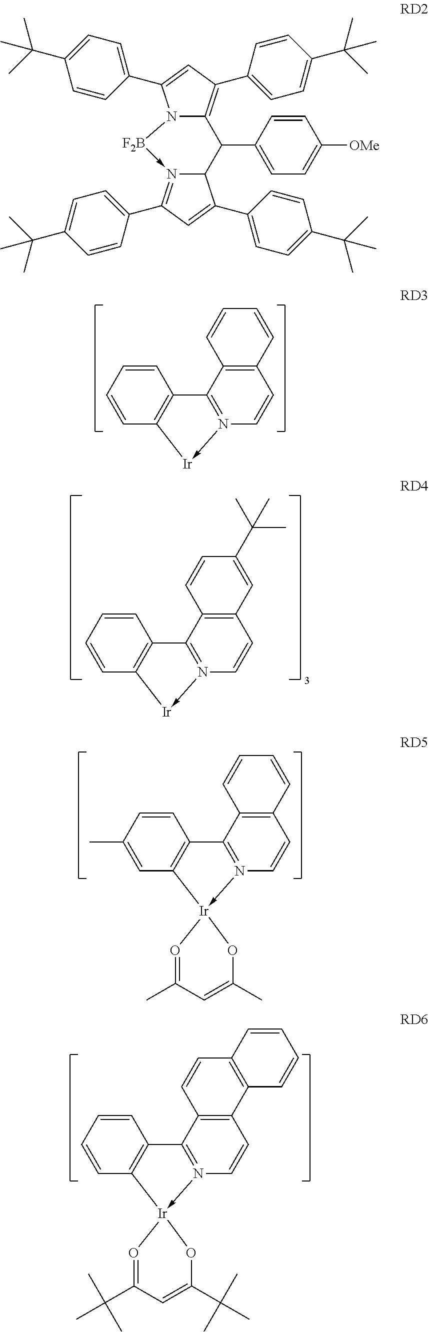

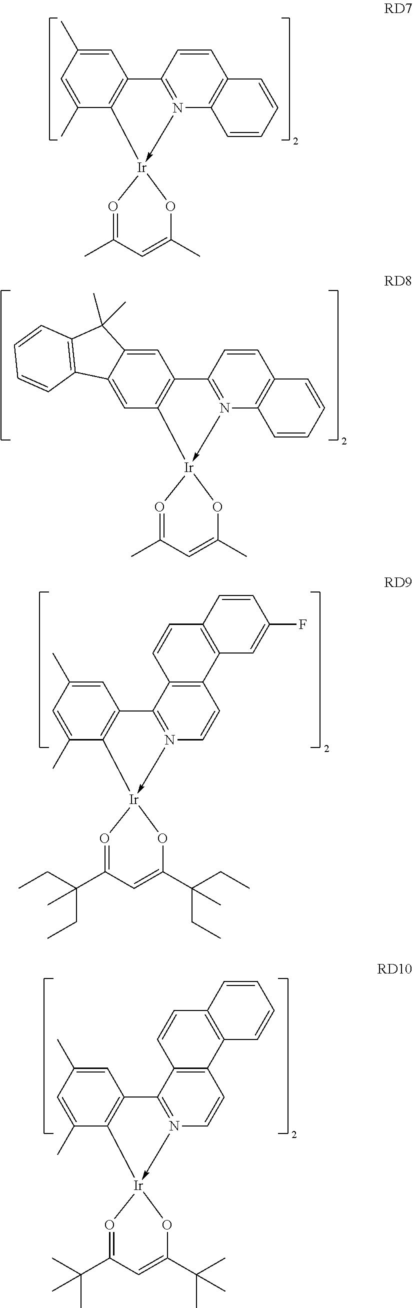

In addition to the compound according to the present embodiment, a host material or an assist material in a light-emitting layer is, for example, an aromatic hydrocarbon compound or a derivative thereof, a carbazole derivative, a dibenzofuran derivative, a dibenzothiophene derivative, an organoaluminum complex, such as tris(8-quinolinolato)aluminum, an organoberyllium complex, or the like. Specific examples of a compound that can be used as a light-emitting layer host or a light-emitting assist material in a light-emitting layer include, but are not limited to, the following.

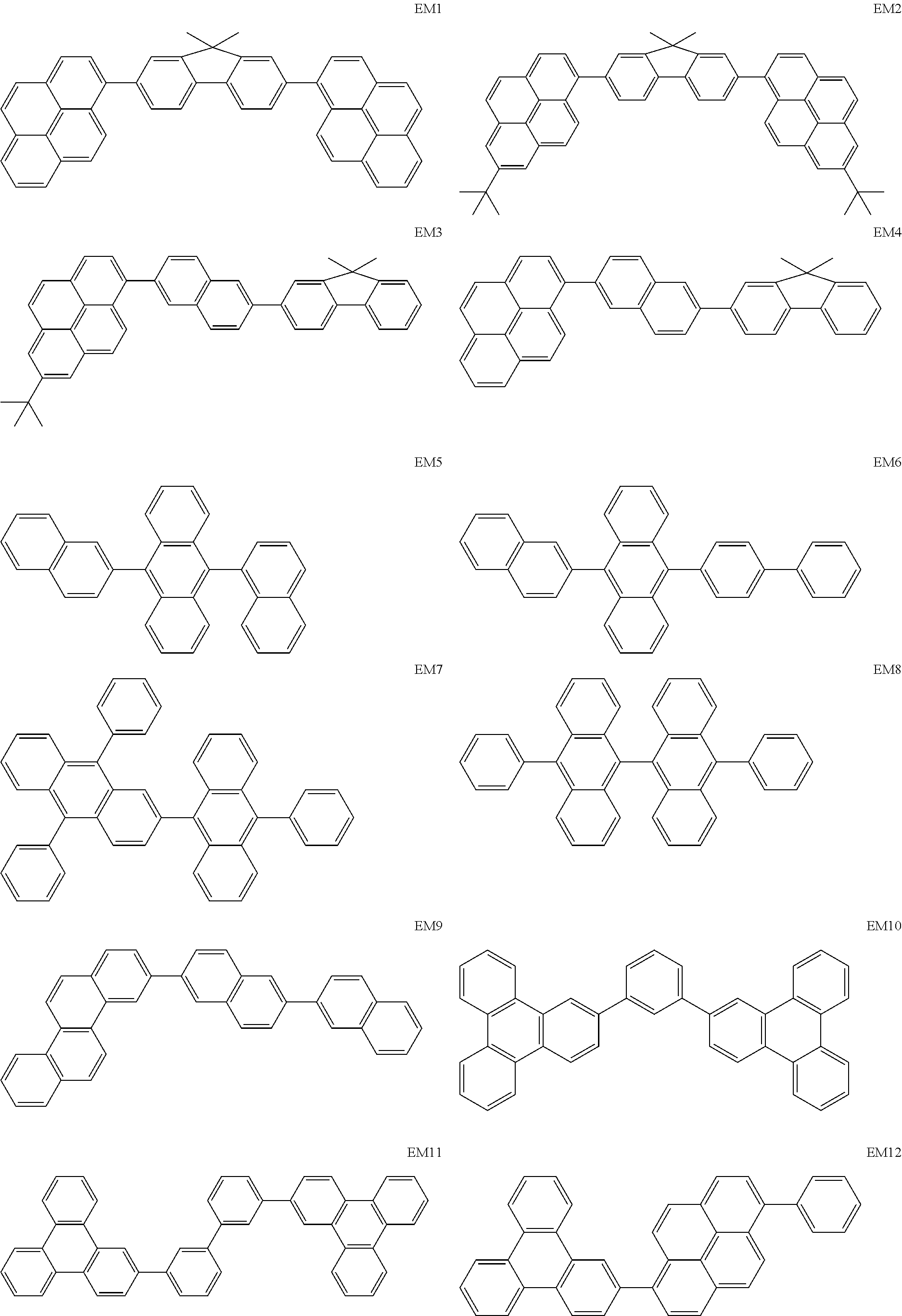

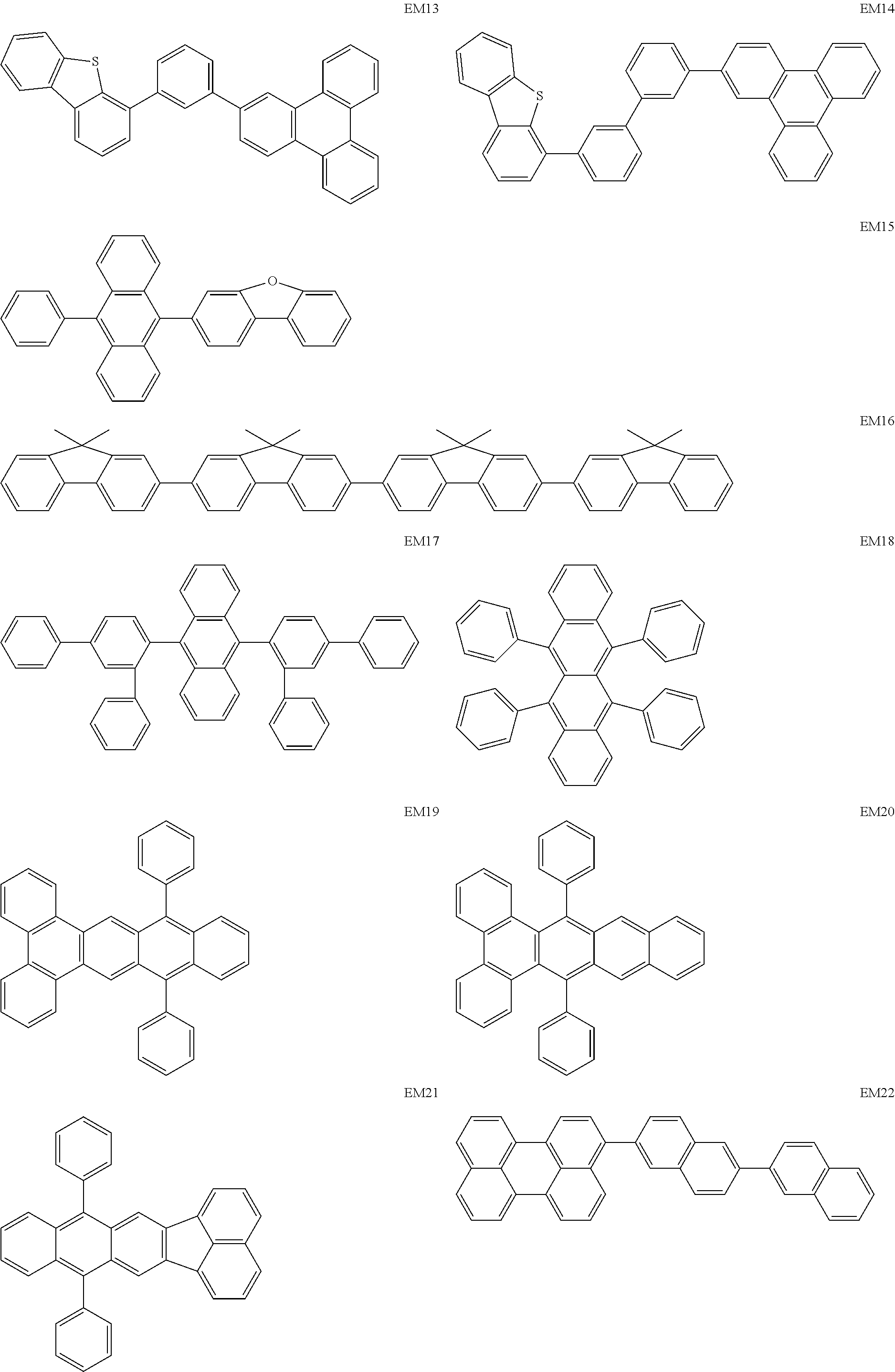

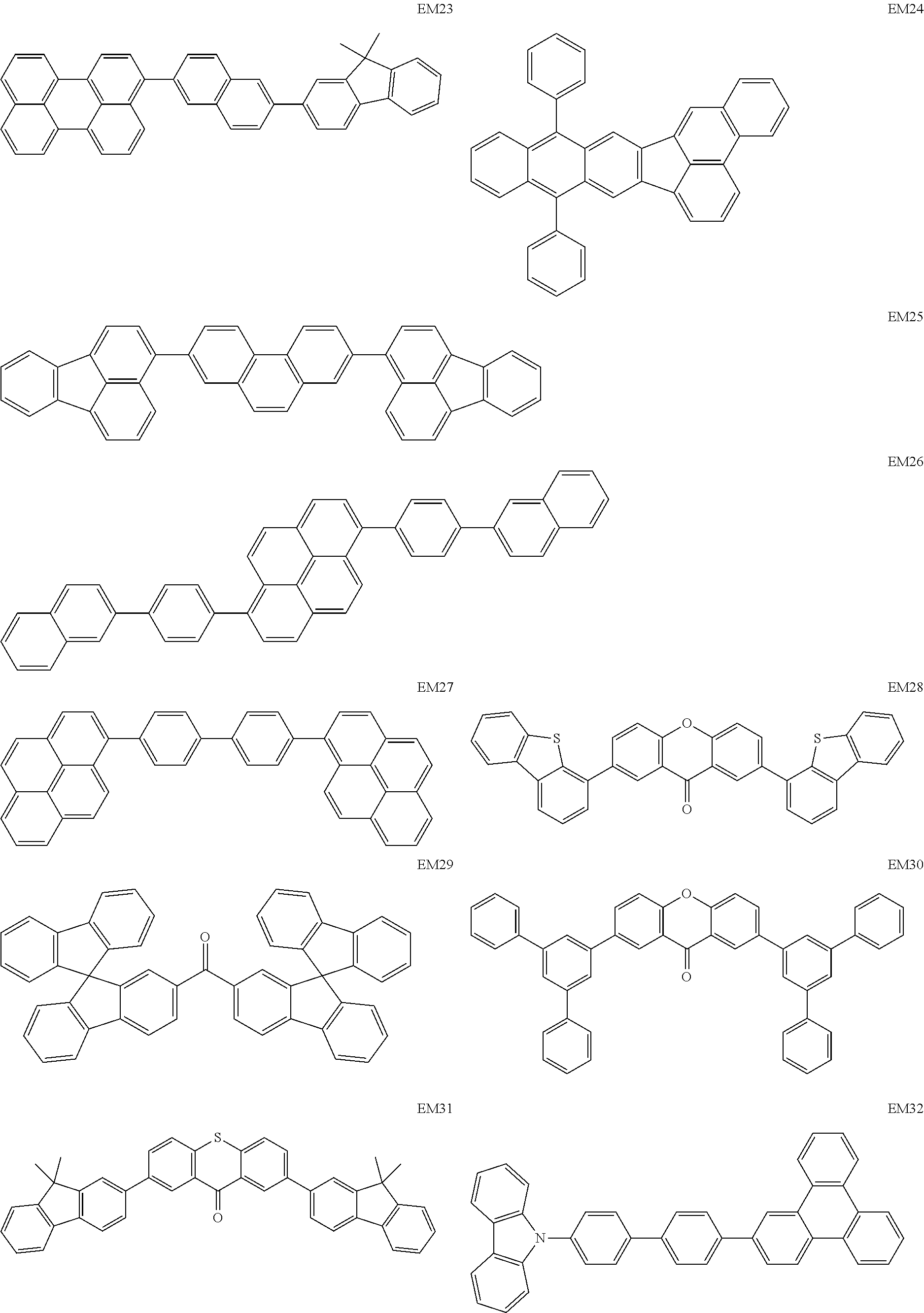

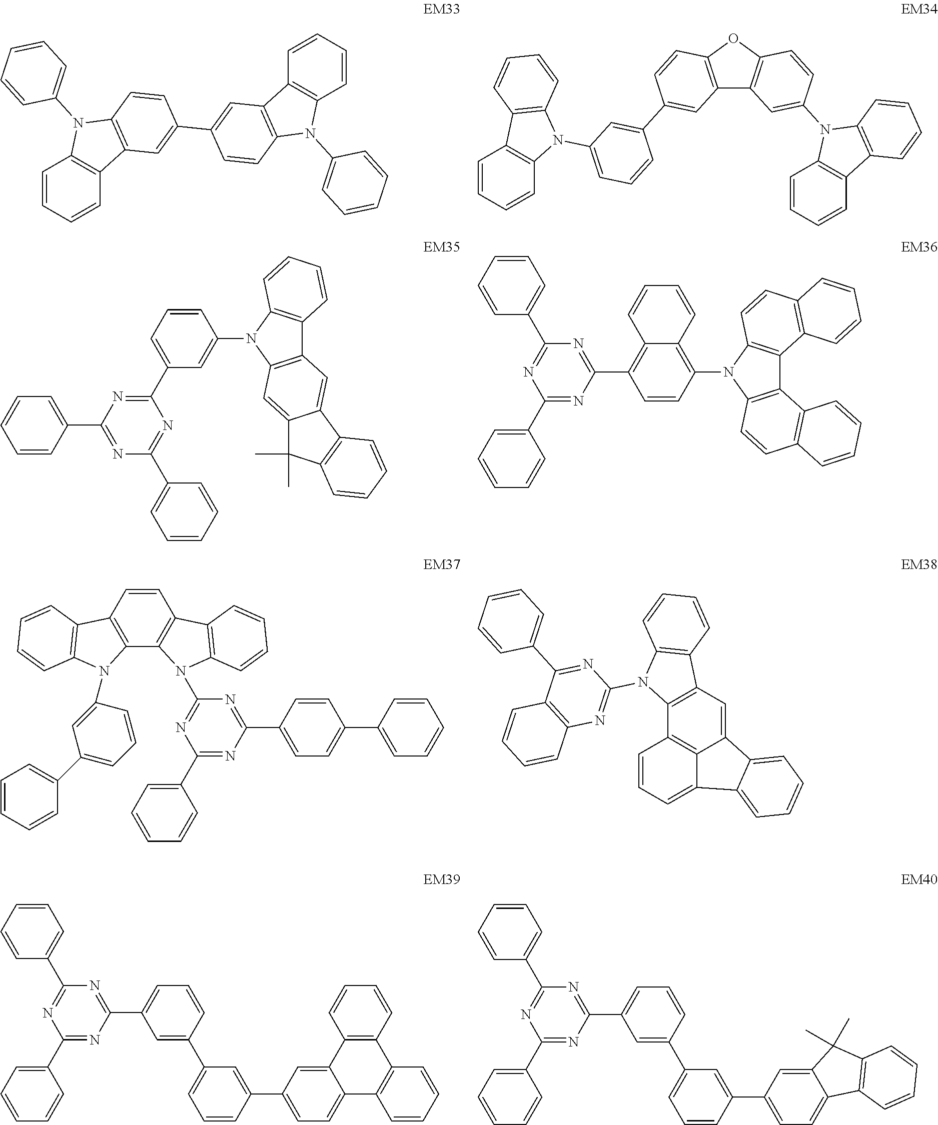

The assist material can be a compound with a triphenylene skeleton or a carbazole skeleton and with good hole transport properties. Among these specific examples, EM10 to EM14, EM32 to EM34, EM13, and EM14 can particularly be used.

An electron transport material can be selected from materials that can transport an electron injected from the negative electrode to the light-emitting layer and is selected in consideration of the balance with the hole mobility of a hole transport material and the like. A material with electron transport ability is, for example, an oxadiazole derivative, an oxazole derivative, a pyrazine derivative, a triazole derivative, a triazine derivative, a quinoline derivative, a quinoxaline derivative, a phenanthroline derivative, an organoaluminum complex, a fused-ring compound (for example, a fluorene derivative, a naphthalene derivative, a chrysene derivative, an anthracene derivative, or the like), or the like. Furthermore, the electron transport material is also suitable for use in a hole-blocking layer. Specific examples of a compound that can be used as an electron transport material include, but are not limited to, the following.

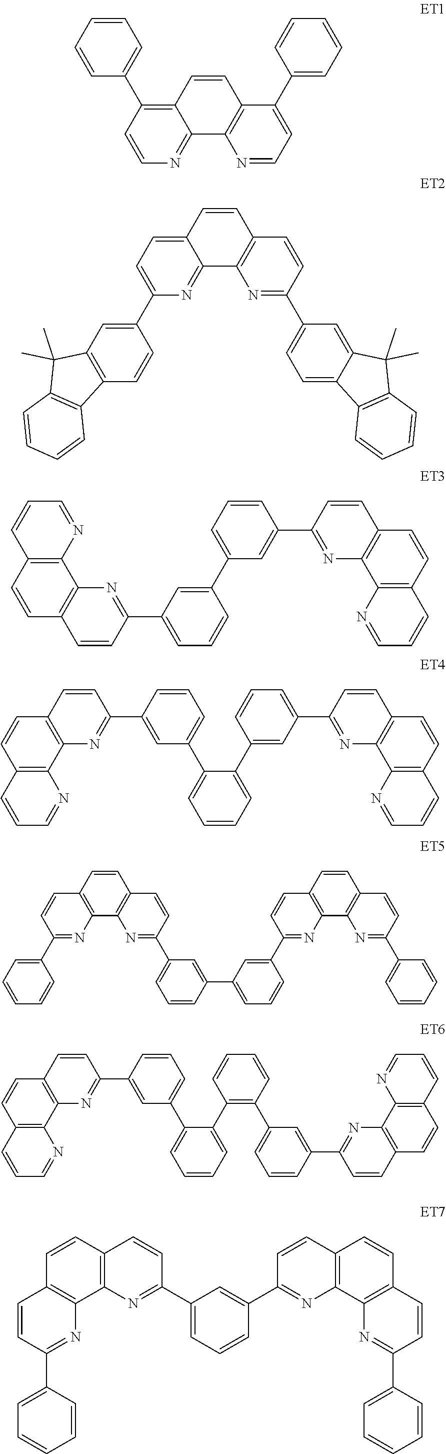

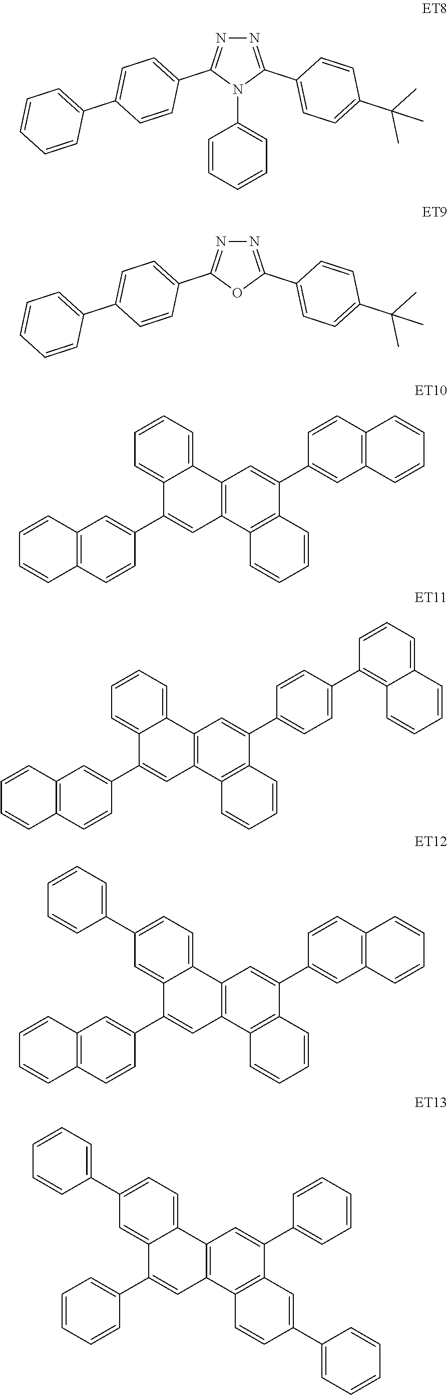

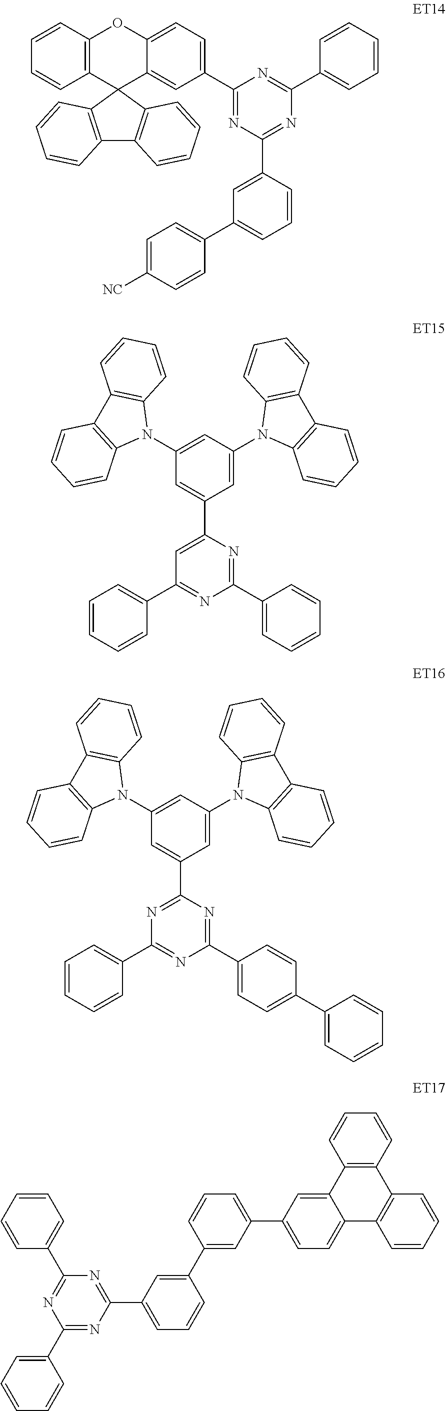 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1 ; c1ccc(-c2cc(-c3cc(-n4c5ccccc5c5ccccc54)cc(-n4c5ccccc5c5ccccc54)c3)nc(-c3ccccc3)n2)cc1 ; c1ccc(-c2ccc(-c3nc(-c4ccccc4)nc(-c4cc(-n5c6ccccc6c6ccccc65)cc(-n5c6ccccc6c6ccccc65)c4)n3)cc2)cc1 ; c1ccc(-c2nc(-c3ccccc3)nc(-c3cccc(-c4cccc(-c5ccc6c7ccccc7c7ccccc7c6c5)c4)c3)n2)cc1]

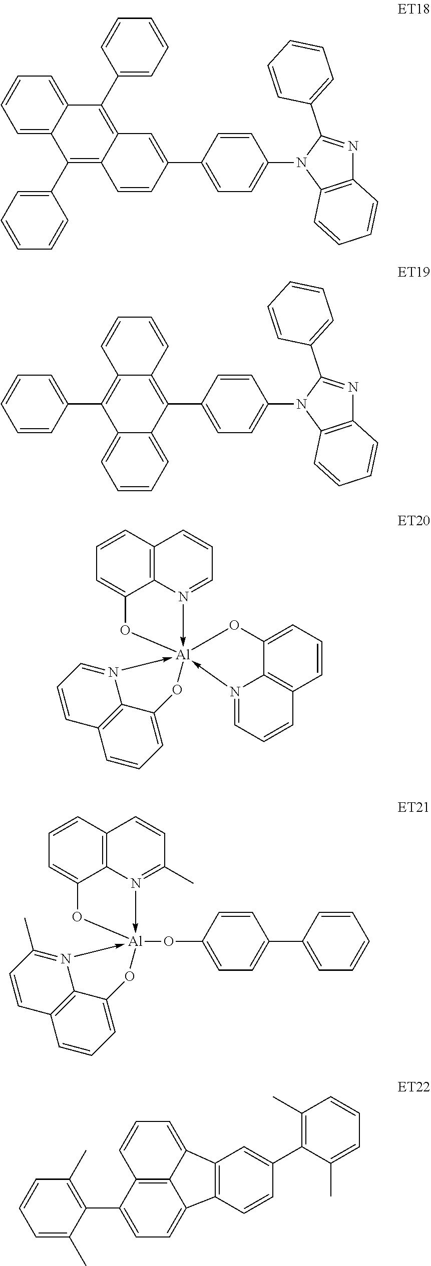

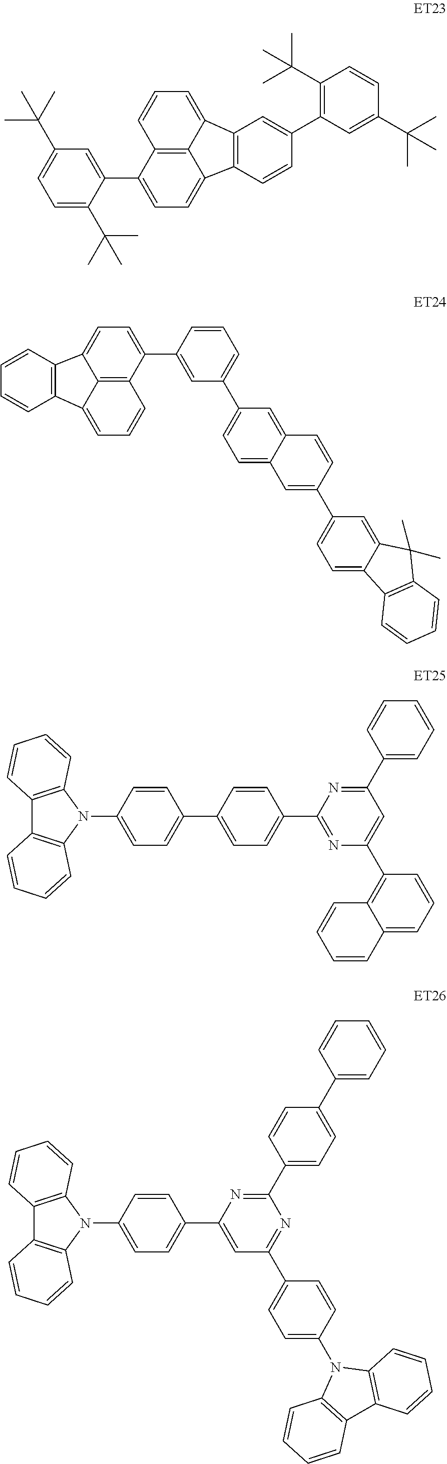

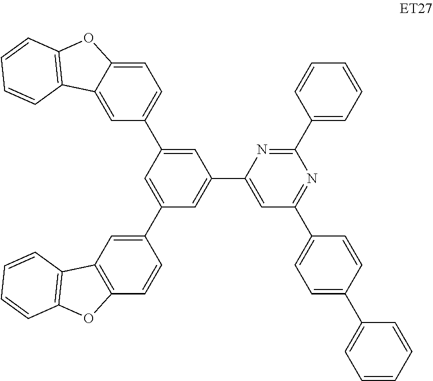 [SMILES: c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

An electron injection material can be selected from materials that can easily inject an electron from the negative electrode and is selected in consideration of the balance with the hole injection properties and the like. An n-type dopant or a reducing dopant may be contained as an organic compound. Examples thereof include a compound containing an alkali metal, such as lithium fluoride, a lithium complex, such as lithium quinolinol, a benzimidazolidene derivative, an imidazolidene derivative, a fulvalene derivative, and an acridine derivative. It can also be used in combination with the electron transport material.

### <Structure of Organic Light-Emitting Device>

An organic light-emitting device includes a first electrode, an organic compound layer, and a second electrode on a substrate. An insulating layer may be provided on the substrate. A protective layer, a color filter, a microlens, or the like may be provided on the second electrode. When a color filter is provided, a planarization layer may be provided between the color filter and the protective layer. The planarization layer may be composed of an acrylic resin or the like. The same applies to the planarization layer provided between the color filter and the microlens. One of the first electrode and the second electrode may be a positive electrode, and the other may be a negative electrode.

### [Substrate]

The substrate may be formed of quartz, glass, a silicon wafer, resin, metal, or the like. The substrate may have a switching device, such as a transistor, and wiring, on which an insulating layer may be provided. The insulating layer may be composed of any material, provided that the insulating layer can have a contact hole for wiring between the insulating layer and the first electrode and is insulated from unconnected wires. For example, the insulating layer may be formed of a resin, such as polyimide, silicon oxide, or silicon nitride.

### [Electrode]

A pair of electrodes can be used as electrodes. The pair of electrodes may be a positive electrode and a negative electrode. When an electric field is applied in a direction in which the organic light-emitting device emits light, an electrode with a high electric potential is a positive electrode, and the other electrode is a negative electrode. In other words, the electrode that supplies a hole to the light-emitting layer is a positive electrode, and the electrode that supplies an electron to the light-emitting layer is a negative electrode.

A constituent material of the positive electrode can have as large a work function as possible. Examples thereof include a metal element, such as gold, platinum, silver, copper, nickel, palladium, cobalt, selenium, vanadium, or tungsten, a mixture thereof, an alloy thereof, and a metal oxide, such as tin oxide, zinc oxide, indium oxide, indium tin oxide (ITO), or indium zinc oxide. An electrically conductive polymer, such as polyaniline, polypyrrole, or polythiophene, may also be used.

These electrode materials may be used alone or in combination. The positive electrode may be composed of a single layer or a plurality of layers.

When used as a reflective electrode, for example, chromium, aluminum, silver, titanium, tungsten, molybdenum, an alloy thereof, or a laminate thereof can be used. These materials can also function as a reflective film that does not have a role as an electrode. When used as a transparent electrode, an oxide transparent electroconductive layer, such as indium tin oxide (ITO) or indium zinc oxide, can be used. However, the present disclosure is not limited thereto. The electrodes may be formed by photolithography.

On the other hand, a constituent material of the negative electrode can be a material with a small work function. For example, an alkali metal, such as lithium, an alkaline-earth metal, such as calcium, a metal element, such as aluminum, titanium, manganese, silver, lead, or chromium, or a mixture thereof may be used. An alloy of these metal elements may also be used. For example, magnesium-silver, aluminum-lithium, aluminum-magnesium, silver-copper, or zinc-silver may be used. A metal oxide, such as indium tin oxide (ITO), may also be used. These electrode materials may be used alone or in combination. The negative electrode may be composed of a single layer or a plurality of layers. In particular, silver can be used, and a silver alloy can be used to reduce the aggregation of silver. As long as the aggregation of silver can be reduced, the alloy may have any ratio. For example, the ratio of silver to another metal may be 1:1, 3:1, or the like.

The negative electrode may be, but is not limited to, an oxide electroconductive layer, such as ITO, for a top emission device or a reflective electrode, such as aluminum (Al), for a bottom emission device. The negative electrode may be formed by any method. A direct-current or alternating-current sputtering method can achieve good film coverage and easily decrease resistance.

### [Organic Compound Layer]

The organic compound layer may be formed of a single layer or a plurality of layers. Depending on their functions, the plurality of layers may be referred to as a hole injection layer, a hole transport layer, an electron-blocking layer, a light-emitting layer, a hole-blocking layer, an electron transport layer, or an electron injection layer. The organic compound layer is composed mainly of an organic compound and may contain an inorganic atom or an inorganic compound. For example, copper, lithium, magnesium, aluminum, iridium, platinum, molybdenum, zinc, or the like may be contained. The organic compound layer may be located between the first electrode and the second electrode and may be in contact with the first electrode and the second electrode.

An organic compound layer (a hole injection layer, a hole transport layer, an electron-blocking layer, a light-emitting layer, a hole-blocking layer, an electron transport layer, an electron injection layer, or the like) constituting an organic light-emitting device according to an embodiment of the present disclosure is formed by the following method.

An organic compound layer constituting an organic light-emitting device according to an embodiment of the present disclosure can be formed by a dry process, such as a vacuum evaporation method, an ionized deposition method, sputtering, or plasma. Instead of the dry process, it is also possible to use a wet process of forming a layer by a known application method (for example, a spin coating method, a casting method, a microgravure coating method, a gravure coating method, a bar coating method, a roll coating method, a wire bar coating method, a dip coating method, a spray coating method, a screen printing method, a flexographic printing method, an offset printing method, an ink jet printing method, a capillary coating method, a nozzle coating method, an LB method, or the like) using a solution in an appropriate solvent. Among these, a vacuum deposition method, an ionized deposition method, an ink jet printing method, a nozzle coating method, and the like are suitable for the production of an organic light-emitting device with a large area.

A layer formed by a vacuum deposition method, a solution coating method, or the like undergoes little crystallization or the like and has high temporal stability. When a film is formed by a coating method, the film may also be formed in combination with an appropriate binder resin.

The binder resin may be, but is not limited to, a polyvinylcarbazole resin, a polycarbonate resin, a polyester resin, an ABS resin, an acrylic resin, a polyimide resin, a phenolic resin, an epoxy resin, a silicone resin, or a urea resin.

These binder resins may be used alone or in combination as a homopolymer or a copolymer. If necessary, an additive agent, such as a known plasticizer, oxidation inhibitor, and/or ultraviolet absorbent, may also be used.

In general, each layer of the organic light-emitting device preferably has a thickness of 1 nm or more and 10 μm or less. In particular, the light-emitting layer of the organic compound layer preferably has a thickness of 10 nm or more and 100 nm or less to achieve effective emission properties.

### [Protective Layer]

A protective layer may be provided on the second electrode. For example, a glass sheet with a moisture absorbent may be attached to the second electrode to decrease the amount of water or the like entering the organic compound layer and to reduce the occurrence of display defects. In another embodiment, a passivation film of silicon nitride or the like may be provided on the second electrode to decrease the amount of water or the like entering the organic compound layer. For example, the second electrode may be formed and then transferred to another chamber without breaking the vacuum, and a silicon nitride film with a thickness of 2 μm may be formed as a protective layer by a chemical vapor deposition (CVD) method. The film formation by the CVD method may be followed by the formation of a protective layer by an atomic layer deposition (ALD) method. A film formed by the ALD method may be formed of any material, such as silicon nitride, silicon oxide, or aluminum oxide. Silicon nitride may be further formed by the CVD method on the film formed by the ALD method. The film formed by the ALD method may have a smaller thickness than the film formed by the CVD method. More specifically, the thickness may be 50% or less or even 10% or less.

### [Color Filter]

A color filter may be provided on the protective layer. For example, a color filter that matches the size of the organic light-emitting device may be provided on another substrate and may be bonded to the substrate on which the organic light-emitting device is provided, or a color filter may be patterned on the protective layer by photolithography. The color filter may be composed of a polymer.

### [Planarization Layer]

A planarization layer may be provided between the color filter and the protective layer. The planarization layer is provided to reduce the roughness of the underlayer. The planarization layer is sometimes referred to as a material resin layer with any purpose. The planarization layer may be composed of an organic compound and can be composed of a high-molecular-weight compound, though it may be composed of a low-molecular-weight compound.

The planarization layer may be provided above and below the color filter, and the constituent materials thereof may be the same or different. Specific examples include a polyvinylcarbazole resin, a polycarbonate resin, a polyester resin, an ABS resin, an acrylic resin, a polyimide resin, a phenolic resin, an epoxy resin, a silicone resin, and a urea resin.

### [Microlens]

An organic light-emitting device may include an optical member, such as a microlens, on the light output side. The microlens may be composed of an acrylic resin, an epoxy resin, or the like. The microlens may be used to increase the amount of light extracted from the organic light-emitting device and to control the direction of the extracted light. The microlens may have a hemispherical shape. For a hemispherical microlens, the vertex of the microlens is a contact point between the hemisphere and a tangent line parallel to the insulating layer among the tangent lines in contact with the hemisphere. The vertex of the microlens in any cross-sectional view can be determined in the same manner. More specifically, the vertex of the microlens in a cross-sectional view is a contact point between the semicircle of the microlens and a tangent line parallel to the insulating layer among the tangent lines in contact with the semicircle.

The midpoint of the microlens can also be defined. In a cross section of the microlens, a midpoint of a line segment from one end point to the other end point of the arc can be referred to as a midpoint of the microlens. A cross section in which the vertex and the midpoint are determined may be perpendicular to the insulating layer.

### [Opposite Substrate]

An opposite substrate may be provided on the planarization layer. The opposite substrate is so called because it faces the substrate. The opposite substrate may be composed of the same material as the substrate. When the substrate is a first substrate, the opposite substrate may be a second substrate.

### [Pixel Circuit]

An organic light-emitting apparatus including an organic light-emitting device may include a pixel circuit coupled to the organic light-emitting device. The pixel circuit may be of an active matrix type, which independently controls the light emission of a first light-emitting device and a second light-emitting device. The active-matrix circuit may be voltage programmed or current programmed. The drive circuit has a pixel circuit for each pixel. The pixel circuit may include a light-emitting device, a transistor for controlling the luminous brightness of the light-emitting device, a transistor for controlling light emission timing, a capacitor for holding the gate voltage of the transistor for controlling the luminous brightness, and a transistor for GND connection without through the light-emitting device.

A light-emitting apparatus includes a display region and a peripheral region around the display region. The display region includes the pixel circuit, and the peripheral region includes a display control circuit. The mobility of a transistor constituting the pixel circuit may be smaller than the mobility of a transistor constituting the display control circuit. The gradient of the current-voltage characteristics of a transistor constituting the pixel circuit may be smaller than the gradient of the current-voltage characteristics of a transistor constituting the display control circuit. The gradient of the current-voltage characteristics can be determined by so-called Vg-Ig characteristics. A transistor constituting the pixel circuit is a transistor coupled to a light-emitting device, such as a first light-emitting device.

### [Pixel]

An organic light-emitting apparatus including an organic light-emitting device may have a plurality of pixels. Each pixel has subpixels that emit light of different colors. For example, the subpixels may have RGB emission colors.

In each pixel, a region also referred to as a pixel aperture emits light. This region is the same as the first region. The pixel aperture may be 15 μm or less or 5 μm or more. More specifically, the pixel aperture may be 11 μm, 9.5 μm, 7.4 μm, or 6.4 μm. The distance between the subpixels may be 10 μm or less, more specifically, 8 μm, 7.4 μm, or 6.4 μm.

The pixels may be arranged in a known form in a plan view. Examples include a stripe arrangement, a delta arrangement, a PenTile arrangement, and a Bayer arrangement. Each subpixel may have any known shape in a plan view. Examples include quadrangles, such as a rectangle and a rhombus, and a hexagon. As a matter of course, the rectangle also includes a figure that is not strictly rectangular but is close to rectangular. The shape of each subpixel and the pixel array can be used in combination.

### <Applications of Organic Light-Emitting Device>

The organic light-emitting device according to the present embodiment can be used as a constituent of a display apparatus or a lighting apparatus. Other applications include an exposure light source for an electrophotographic image-forming apparatus, a backlight for a liquid crystal display, and a light-emitting apparatus with a color filter in a white light source.

The display apparatus may be an image-information-processing apparatus that includes an image input unit for inputting image information from an area CCD, a linear CCD, a memory card, or the like, includes an information processing unit for processing the input information, and displays an input image on a display unit. The display apparatus may have a plurality of pixels, and at least one of the pixels may include the organic light-emitting device according to the present embodiment and an active device, such as a transistor, coupled to the organic light-emitting device. The substrate may be a semiconductor substrate formed of silicon or the like, and the transistor may be a MOSFET formed on the substrate. The image display apparatus includes an input portion for inputting image information and a display unit for outputting an image, and the display unit includes the display apparatus according to the present embodiment.

A display unit of an imaging apparatus or an ink jet printer may have a touch panel function. A driving system of the touch panel function may be, but is not limited to, an infrared radiation system, an electrostatic capacitance system, a resistive film system, or an electromagnetic induction system. The display apparatus may be used for a display unit of a multifunction printer.

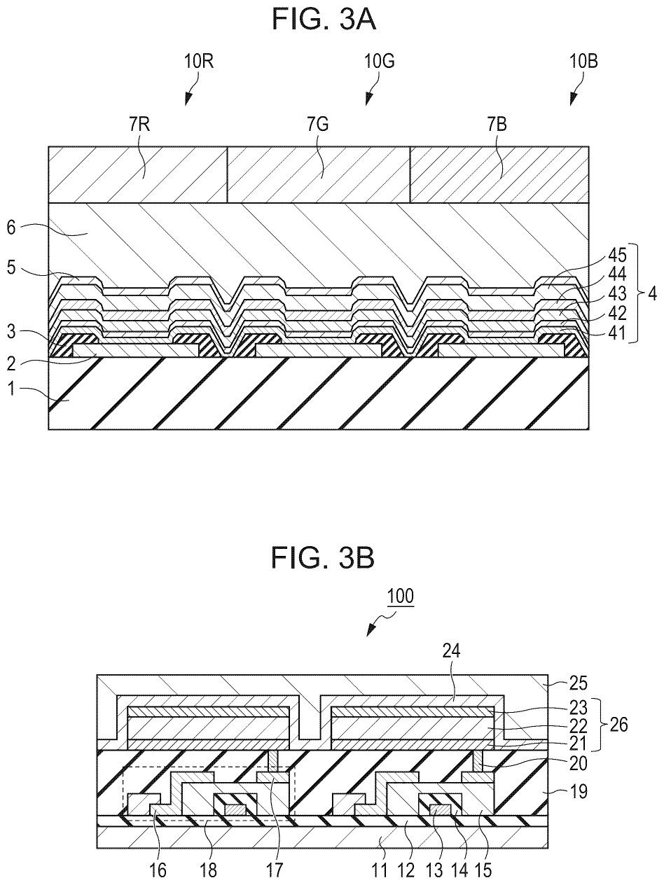

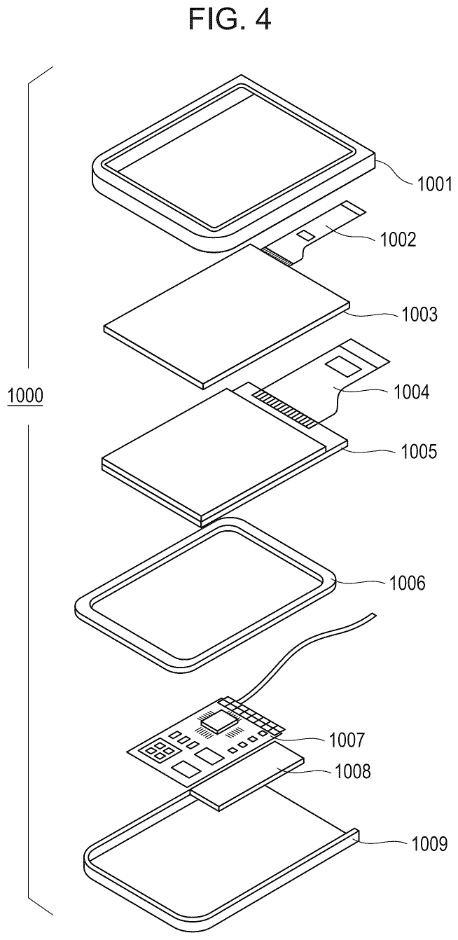

Next, the display apparatus according to the present embodiment is described below with reference to the accompanying drawings. FIGS. 3A and 3B are schematic cross-sectional views of an example of a display apparatus that includes an organic light-emitting device and a transistor coupled to the organic light-emitting device. The transistor is an example of an active device. The transistor may be a thin-film transistor (TFT).

FIG. 3A is a schematic cross-sectional view of an example of a pixel serving as a constructional element of the display apparatus according to the present embodiment. The pixel has subpixels 10. The subpixels are 10R, 10G, and 10B with different emission colors. The emission colors may be distinguished by the wavelength of light emitted from the light-emitting layer, or light emitted from each subpixel may be selectively transmitted or color-converted with a color filter or the like. Each of the subpixels 10 includes a reflective electrode serving as a first electrode 2, an insulating layer 3 covering an end of the first electrode 2, organic compound layers 4 covering the first electrode 2 and the insulating layer 3, a transparent electrode serving as a second electrode 5, a protective layer 6, and a color filter 7 on an interlayer insulating layer 1.

A transistor and/or a capacitor device may be provided under or inside the interlayer insulating layer 1. The transistor and the first electrode 2 may be electrically connected via a contact hole (not shown) or the like.

The insulating layer 3 is also referred to as a bank or a pixel separation film. The insulating layer 3 covers the ends of the first electrode 2 and surrounds the first electrode 2. A portion not covered with the insulating layer 3 is in contact with the organic compound layers 4 and serves as a light-emitting region.

The organic compound layers 4 include a hole injection layer 41, a hole transport layer 42, a light-emitting layer 43, a hole-blocking layer 44, and an electron transport layer 45.

The second electrode 5 may be a transparent electrode, a reflective electrode, or a semitransparent electrode. The protective layer 6 reduces the penetration of moisture into the organic compound layers 4. The protective layer 6 is illustrated as a single layer but may be a plurality of layers. The protective layer 6 may include an inorganic compound layer and an organic compound layer.

The color filter 7 is divided into 7R, 7G, and 7B according to the color. The color filter 7 may be formed on a planarization film (not shown). Furthermore, a resin protective layer (not shown) may be provided on the color filter 7. The color filter 7 may be formed on the protective layer 6. Alternatively, the color filter 7 may be bonded after being provided on an opposite substrate, such as a glass substrate.

A display apparatus 100 illustrated in FIG. 3B includes an organic light-emitting device 26 and a TFT 18, which is an example of a transistor. The display apparatus includes a substrate 11 made of glass, silicon, or the like and an insulating layer 12 on the substrate 11. An active device, such as the TFT 18, and a gate electrode 13, a gate-insulating film 14, and a semiconductor layer 15 of the active device are disposed on the insulating layer 12. The TFT 18 has a drain electrode 16 and a source electrode 17. The TFT 18 is covered with an insulating film 19. A positive electrode 21 of the organic light-emitting device 26 is coupled to the source electrode 17 through a contact hole 20 formed in the insulating film 19.

Electrical connection between the electrodes of the organic light-emitting device 26 (the positive electrode 21 and a negative electrode 23) and the electrodes of the TFT 18 (the source electrode 17 and the drain electrode 16) is not limited to that illustrated in FIG. 3B. More specifically, it is only necessary to electrically connect either the positive electrode 21 or the negative electrode 23 to either the source electrode 17 or the drain electrode 16 of the TFT 18.

Although an organic compound layer 22 is a single layer in the display apparatus 100 illustrated in FIG. 3B, the organic compound layer 22 may be composed of a plurality of layers. The negative electrode 23 is covered with a first protective layer 24 and a second protective layer 25 for reducing degradation of the organic light-emitting device 26.

The transistor used as a switching device in the display apparatus 100 illustrated in FIG. 3B may be replaced with another switching device, such as a metal insulator metal (MIM) device.

The transistor used in the display apparatus 100 in FIG. 3B is not limited to a thin-film transistor including an active layer on an insulating surface of a substrate and may also be a transistor including a single crystal silicon wafer. The active layer may be single-crystal silicon, non-single-crystal silicon, such as amorphous silicon or microcrystalline silicon, or a non-single-crystal oxide semiconductor, such as indium zinc oxide or indium gallium zinc oxide. The thin-film transistor is also referred to as a TFT device.

The transistor in the display apparatus 100 of FIG. 3B may be formed within a substrate, such as a Si substrate. The phrase “formed within a substrate” means that the substrate, such as a Si substrate, itself is processed to form the transistor. Thus, the transistor within the substrate can be considered that the substrate and the transistor are integrally formed.

In the organic light-emitting device according to the present embodiment, the luminous brightness is controlled with the TFT, which is an example of a switching device. The organic light-emitting device can be provided in a plurality of planes to display an image at each luminous brightness. The switching device according to the present embodiment is not limited to the TFT and may be a transistor formed of low-temperature polysilicon or an active-matrix driver formed on a substrate, such as a Si substrate. The phrase “on a substrate” may also be referred to as “within a substrate”. Whether a transistor is provided within a substrate or a TFT is used depends on the size of a display unit. For example, for an approximately 0.5-inch display unit, an organic light-emitting device can be provided on a Si substrate.

FIG. 4 is a schematic view of an example of the display apparatus according to the present embodiment. A display apparatus 1000 may include a touch panel 1003, a display panel 1005, a frame 1006, a circuit substrate 1007, and a battery 1008 between an upper cover 1001 and a lower cover 1009. The touch panel 1003 and the display panel 1005 are coupled to flexible print circuits FPC 1002 and 1004, respectively. Transistors are printed on the circuit substrate 1007. The battery 1008 is not necessarily provided when the display apparatus is not a mobile device, or may be provided at another position even when the display apparatus is a mobile device.

The display apparatus according to the present embodiment may include color filters of red, green, and blue colors. In the color filters, the red, green, and blue colors may be arranged in a delta arrangement.

The display apparatus according to the present embodiment may be used for a display unit of a mobile terminal. Such a display apparatus may have both a display function and an operation function. The mobile terminal may be a mobile phone, such as a smartphone, a tablet, a head-mounted display, or the like.

The display apparatus according to the present embodiment may be used for a display unit of an imaging apparatus that includes an optical unit with a plurality of lenses and an imaging device for receiving light passing through the optical unit. The imaging apparatus may include a display unit for displaying information acquired by the imaging device. The display unit may be a display unit exposed outside from the imaging apparatus or a display unit located in a finder. The imaging apparatus may be a digital camera or a digital video camera.

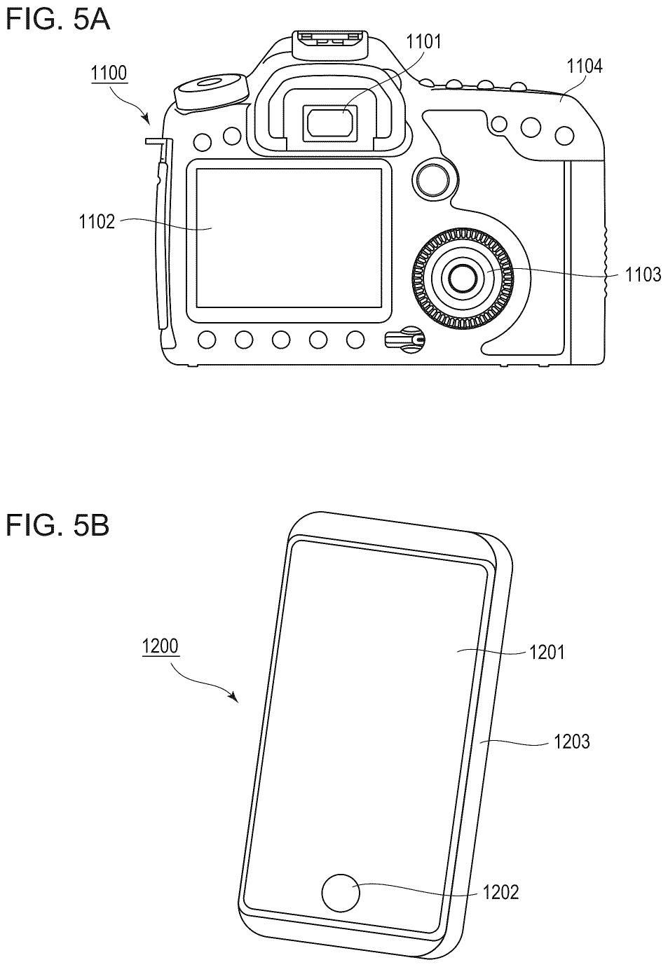

FIG. 5A is a schematic view of an example of an imaging apparatus according to the present embodiment. An imaging apparatus 1100 may include a viewfinder 1101, a rear display 1102, an operating unit 1103, and a housing 1104. The viewfinder 1101 may include the display apparatus according to the present embodiment. In such a case, the display apparatus may display environmental information, imaging instructions, and the like as well as an image to be captured. The environmental information may include the intensity of external light, the direction of external light, the travel speed of the photographic subject, the possibility that the photographic subject is shielded by a shielding material, and the like.

Because the appropriate timing for imaging is a short time, it is better to display information as early as possible. Thus, a display apparatus including an organic light-emitting device according to the present embodiment can be used. This is because the organic light-emitting device has a high response speed. A display apparatus including the organic light-emitting device requires a high display speed and can be more suitably used than liquid crystal displays.

The imaging apparatus 1100 includes an optical unit (not shown). The optical unit has a plurality of lenses and focuses an image on an imaging device in the housing 1104. The focus of the lenses can be adjusted by adjusting their relative positions. This operation can also be automatically performed. The imaging apparatus may also be referred to as a photoelectric conversion apparatus. The photoelectric conversion apparatus can have, as an imaging method, a method of detecting a difference from a previous image, a method of cutting out a permanently recorded image, or the like, instead of taking an image one after another.

FIG. 5B is a schematic view of an example of electronic equipment according to the present embodiment. Electronic equipment 1200 includes a display unit 1201, an operating unit 1202, and a housing 1203. The housing 1203 may include a circuit, a printed circuit board including the circuit, a battery, and a communication unit. The operating unit 1202 may be a button or a touch panel response unit. The operating unit 1202 may be a biometric recognition unit that recognizes a fingerprint and releases the lock. Electronic equipment with a communication unit may also be referred to as communication equipment. The electronic equipment 1200 may have a lens and an imaging device and thereby further have a camera function. An image captured by the camera function is displayed on the display unit 1201. The electronic equipment 1200 may be a smartphone, a notebook computer, or the like.

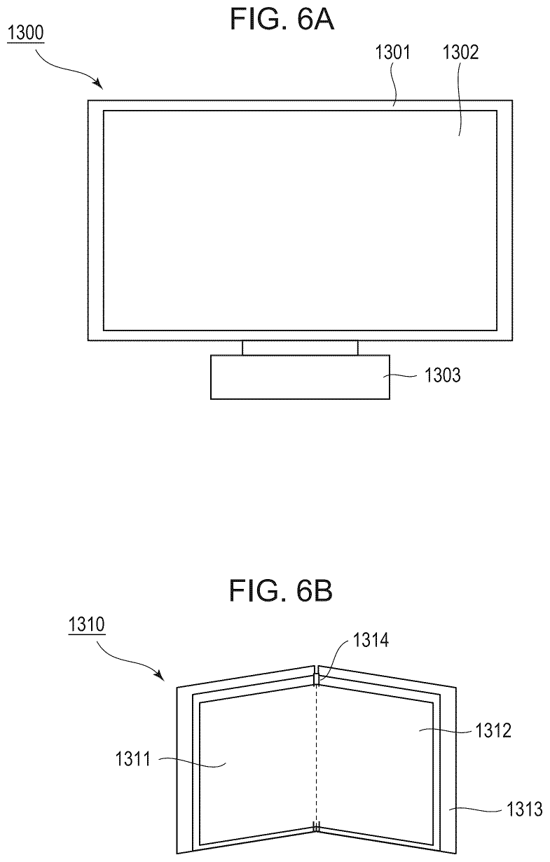

FIGS. 6A and 6B are schematic views of an example of the display apparatus according to the present embodiment. FIG. 6A illustrates a display apparatus, such as a television monitor or a PC monitor. A display apparatus 1300 includes a frame 1301 and a display unit 1302. The light-emitting device according to the present embodiment may be used for the display unit 1302. The display apparatus 1300 includes a base 1303 for supporting the frame 1301 and the display unit 1302. The base 1303 is not limited to the structure illustrated in FIG. 6A. The lower side of the frame 1301 may also serve as the base. The frame 1301 and the display unit 1302 may be bent. The radius of curvature thereof may be 5000 mm or more and 6000 mm or less.

FIG. 6B is a schematic view of another example of the display apparatus according to the present embodiment. A display apparatus 1310 in FIG. 6B is configured to be foldable and is a so-called foldable display apparatus. The display apparatus 1310 includes a first display unit 1311, a second display unit 1312, a housing 1313, and a folding point 1314. The first display unit 1311 and the second display unit 1312 may include the light-emitting device according to the present embodiment. The first display unit 1311 and the second display unit 1312 may be a single display apparatus without a joint. The first display unit 1311 and the second display unit 1312 can be divided by the folding point. The first display unit 1311 and the second display unit 1312 may display different images or one image.

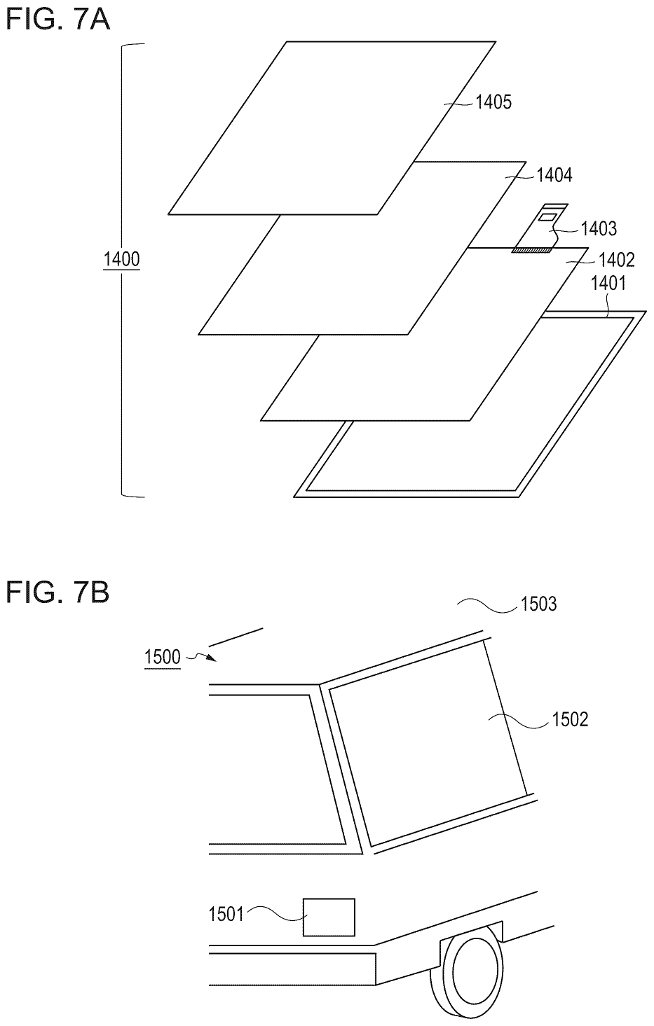

FIG. 7A is a schematic view of an example of a lighting apparatus according to the present embodiment. A lighting apparatus 1400 may include a housing 1401, a light source 1402, a circuit substrate 1403, an optical filter 1404 that transmits light emitted by the light source 1402, and a light-diffusing unit 1405. The light source 1402 may include the organic light-emitting device according to the present embodiment. The optical filter 1404 may be a filter that improves the color rendering properties of the light source. The light-diffusing unit 1405 can effectively diffuse light from the light source and widely spread light as in illumination. The optical filter 1404 and the light-diffusing unit 1405 may be provided on the light output side of the lighting apparatus. If necessary, a cover may be provided on the outermost side.

For example, the lighting apparatus is an interior lighting apparatus. The lighting apparatus may emit white light, neutral white light, or light of any color from blue to red. The lighting apparatus may have a light control circuit for controlling such light or a color control circuit for controlling emission color. The lighting apparatus may include the organic light-emitting device according to the present embodiment and a power supply circuit coupled thereto. The power supply circuit is a circuit that converts an AC voltage to a DC voltage. The lighting apparatus may have an inverter circuit. White has a color temperature of 4200 K, and neutral white has a color temperature of 5000 K. The lighting apparatus may have a color filter.

The lighting apparatus according to the present embodiment may include a heat dissipation unit. The heat dissipation unit releases heat from the apparatus to the outside and may be a metal or liquid silicon with a high specific heat.

FIG. 7B is a schematic view of an automobile as an example of a moving body according to the present embodiment. The automobile has a taillight as an example of a lamp. An automobile 1500 may have a taillight 1501, which comes on when a brake operation or the like is performed.

The taillight 1501 may include the organic light-emitting device according to the present embodiment. The taillight 1501 may include a protective member for protecting the organic light-emitting device. The protective member may be formed of any transparent material with moderately high strength and can be formed of polycarbonate or the like. The polycarbonate may be mixed with a furan dicarboxylic acid derivative, an acrylonitrile derivative, or the like.

The automobile 1500 may have a body 1503 and a window 1502 on the body 1503. The window 1502 may be a transparent display as long as it is not a window for checking the front and rear of the automobile. The transparent display may include the organic light-emitting device according to the present embodiment. In such a case, constituent materials, such as electrodes, of the organic light-emitting device are transparent materials.

The moving body according to the present embodiment may be a ship, an aircraft, a drone, or the like. The moving body may include a body and a lamp provided on the body. The lamp may emit light to indicate the position of the body. The lamp includes the organic light-emitting device according to the present embodiment.

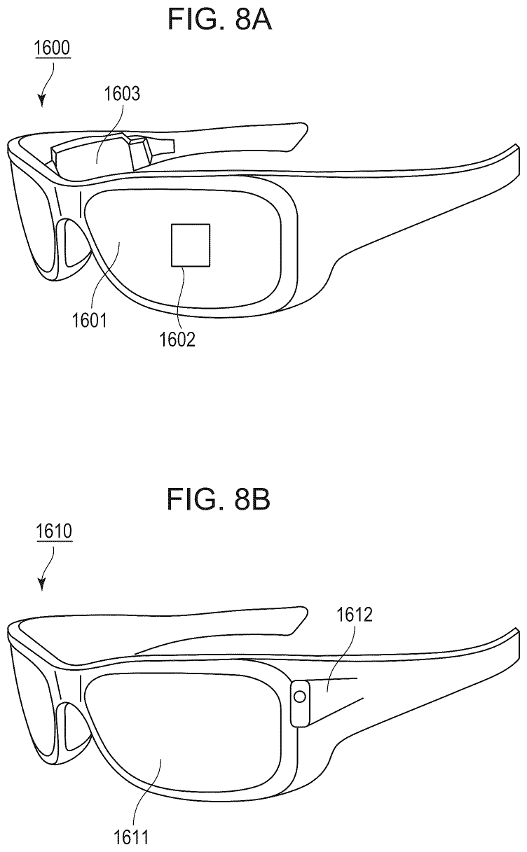

Application examples of the display apparatus according to one of the embodiments are described below with reference to FIGS. 8A and 8B. The display apparatus can be applied to a system that can be worn as a wearable device, such as smart glasses, a head-mounted display (HMD), or smart contact lenses. An imaging and displaying apparatus used in such an application example includes an imaging apparatus that can photoelectrically convert visible light and a display apparatus that can emit visible light.

FIG. 8A is a schematic view of an example of a wearable device according to an embodiment of the present disclosure. Glasses 1600 (smart glasses) according to one application example will be described below with reference to FIG. 8A. An imaging apparatus 1602, such as a complementary metal-oxide semiconductor (CMOS) sensor or a single-photon avalanche photodiode (SPAD), is provided on the front side of a lens 1601 of the glasses 1600. The display apparatus according to one of the embodiments is provided on the back side of the lens 1601.

The glasses 1600 further include a controller 1603. The controller 1603 functions as a power supply for supplying power to the imaging apparatus 1602 and the display apparatus. The controller 1603 controls the operation of the imaging apparatus 1602 and the display apparatus. The lens 1601 has an optical system for focusing light on the imaging apparatus 1602.

FIG. 8B is a schematic view of another example of a wearable device according to an embodiment of the present disclosure. Glasses 1610 (smart glasses) according to one application example will be described below with reference to FIG. 8B. The glasses 1610 have a controller 1612, which includes an imaging apparatus corresponding to the imaging apparatus 1602 of FIG. 8A and a display apparatus. A lens 1611 includes an optical system for projecting light from the imaging apparatus of the controller 1612 and the display apparatus, and an image is projected on the lens 1611. The controller 1612 functions as a power supply for supplying power to the imaging apparatus and the display apparatus and controls the operation of the imaging apparatus and the display apparatus.

The controller 1612 may include a line-of-sight detection unit for detecting the line of sight of the wearer. Infrared radiation may be used to detect the line of sight. An infrared radiation unit emits infrared light to an eyeball of a user who is gazing at a display image. Reflected infrared light from the eyeball is detected by an imaging unit including a light-receiving device to capture an image of the eyeball. A reduction unit for reducing light from the infrared radiation unit to a display unit in a plan view is provided to reduce degradation in image quality. The line of sight of the user for the display image is detected from the image of the eyeball captured by infrared imaging. Any known technique can be applied to line-of-sight detection using the captured image of the eyeball. For example, it is possible to use a line-of-sight detection method based on a Purkinje image obtained by the reflection of irradiation light by the cornea. More specifically, a line-of-sight detection process based on a pupil-corneal reflection method is performed. The line of sight of the user is detected by calculating a line-of-sight vector representing the direction (rotation angle) of an eyeball on the basis of an image of a pupil and a Purkinje image included in a captured image of the eyeball using the pupil-corneal reflection method.

A display apparatus according to an embodiment of the present disclosure may include an imaging apparatus including a light-receiving device and may control a display image on the basis of line-of-sight information of a user from the imaging apparatus. More specifically, on the basis of the line-of-sight information, the display apparatus determines a first visibility region at which the user gazes and a second visibility region other than the first visibility region. The first visibility region and the second visibility region may be determined by the controller of the display apparatus or may be received from an external controller. In the display region of the display apparatus, the first visibility region may be controlled to have higher display resolution than the second visibility region. In other words, the second visibility region may have lower resolution than the first visibility region.

The display region has a first display region and a second display region different from the first display region, and the priority of the first display region and the second display region depends on the line-of-sight information. The first visibility region and the second visibility region may be determined by the controller of the display apparatus or may be received from an external controller. A region with a higher priority may be controlled to have higher resolution than another region. In other words, a region with a lower priority may have lower resolution.

The first visibility region or a region with a higher priority may be determined by artificial intelligence (AI). The AI may be a model configured to estimate the angle of the line of sight and the distance to a target ahead of the line of sight from an image of an eyeball using the image of the eyeball and the direction in which the eyeball actually viewed in the image as teaching data. The AI program may be stored in the display apparatus, the imaging apparatus, or an external device. The AI program stored in an external device is transmitted to the display apparatus via communication.

For display control based on visual recognition detection, the present disclosure can be applied to smart glasses further having an imaging apparatus for imaging the outside. Smart glasses can display captured external information in real time.

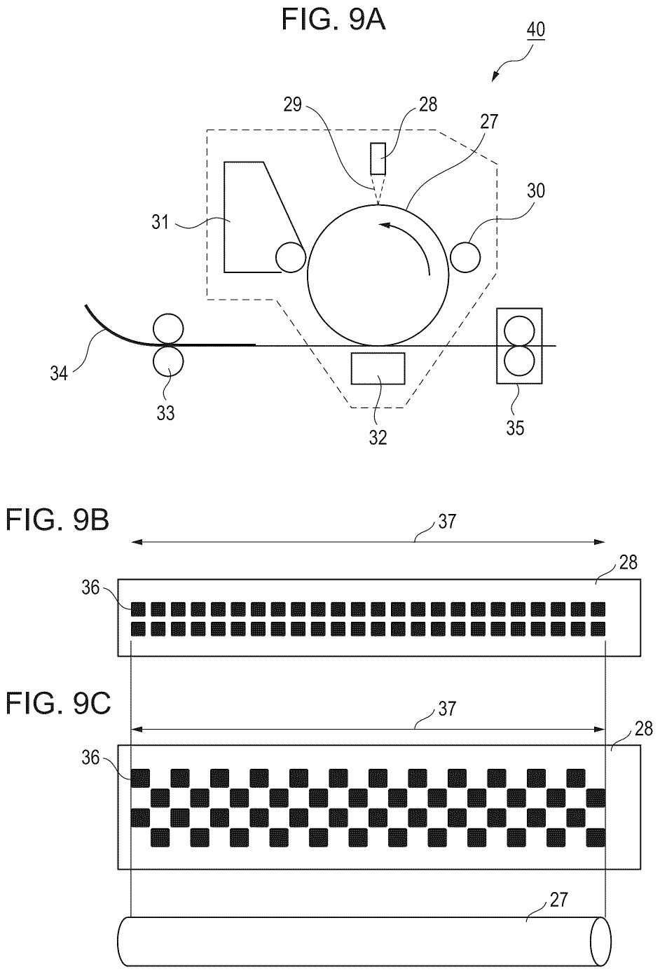

FIG. 9A is a schematic view of an example of an image-forming apparatus according to an embodiment of the present disclosure. An image-forming apparatus 40 is an electrophotographic image-forming apparatus and includes a photosensitive member 27, an exposure light source 28, a charging unit 30, a developing unit 31, a transfer unit 32, a conveying roller 33, and a fixing unit 35. The exposure light source 28 emits light 29, and an electrostatic latent image is formed on the surface of the photosensitive member 27. The exposure light source 28 includes the organic light-emitting device according to the present embodiment. The developing unit 31 contains toner and the like. The charging unit 30 electrifies the photosensitive member 27. The transfer unit 32 transfers a developed image onto a recording medium 34. The conveying roller 33 conveys the recording medium 34. The recording medium 34 is paper, for example. The fixing unit 35 fixes an image on the recording medium 34.

FIGS. 9B and 9C are schematic views of the exposure light source 28, wherein a plurality of light-emitting portions 36 are arranged on a long substrate. An arrow 37 indicates a direction parallel to the axis of the photosensitive member and indicates a column direction in which the organic light-emitting devices are arranged. This column direction is the same as the direction of the rotation axis of the photosensitive member 27. This direction can also be referred to as the major-axis direction of the photosensitive member 27. In FIG. 9B, the light-emitting portions 36 are arranged in the major-axis direction of the photosensitive member 27. In FIG. 9C, unlike FIG. 9B, the light-emitting portions 36 are alternately arranged in the column direction in the first and second columns. The first and second columns are arranged at different positions in the row direction. In the first column, the light-emitting portions 36 are arranged at intervals. In the second column, the light-emitting portions 36 are arranged at positions corresponding to the spaces between the light-emitting portions 36 of the first column. Thus, the light-emitting portions 36 are also arranged at intervals in the row direction. The arrangement in FIG. 9C can also be referred to as a grid-like pattern, a staggered pattern, or a checkered pattern, for example.

As described above, an apparatus including the organic light-emitting device according to the present embodiment can be used to stably display a high-quality image for extended periods. Furthermore, an apparatus including the organic light-emitting device according to the present embodiment can be used to achieve both good visibility outdoors and power-saving display due to high-efficiency and high-luminance light output.

### EXEMPLARY EMBODIMENTS

The present disclosure is described below with exemplary embodiments. However, the present disclosure is not limited to these examples.

### Exemplary Embodiment 1 (Synthesis of Exemplary Compound A1)

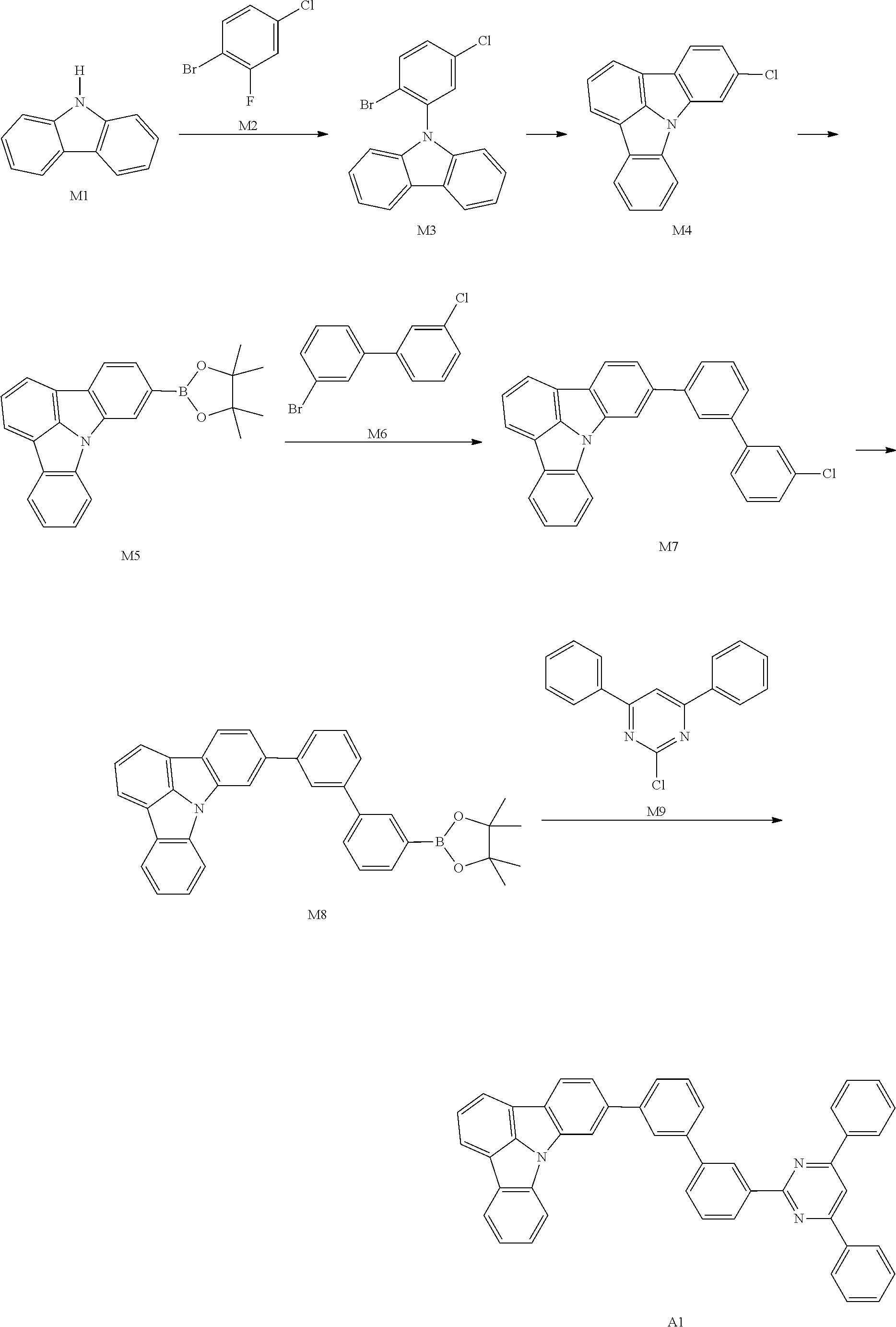

### (1) Synthesis of Compound M3

A 3-L recovery flask was charged with the following reagent(s) and solvent(s).
Compound M1: 71.1 g (425 mmol)
Compound M2: 98.0 g (468 mmol)
Sodium tert-butoxide: 61.3 g (638 mmol)
Dehydrated DMAc: 1.4 L

Next, the reaction solution was heated to 100° C. in a nitrogen stream and was stirred at this temperature (100° C.) for 1 hour. After completion of the reaction, the product was poured into 1.5 L of water and was extracted twice with 1.5 L of ethyl acetate. The organic phases were combined, were washed with 1.0 L of saturated saline, and were dried over anhydrous sodium sulfate. The solution was concentrated to produce 218 g of a white brown solid. This solid was dispersed and washed with 600 mL of ethanol at approximately 5° C. and was then dried under reduced pressure at 80° C. to produce 127 g of a compound M3 as a white solid (yield: 84%).

### (2) Synthesis of Compound M4

A 3-L recovery flask was charged with the following reagent(s) and solvent(s).
Compound M3: 126 g (353 mmol)
Diazabicycloundecene: 161 g (353 mmol)
Dehydrated DMAc: 1.8 L

The reaction solution was then degassed, and 7.44 g (10.6 mmol) of bis(triphenylphosphine) palladium (II) dichloride was added thereto. The reaction liquid was stirred overnight at an internal temperature of 130° C. and was further stirred at 140° C. for 2 hours. The disappearance of the raw material compound M3 was then confirmed. The brown suspension was cooled and was then poured into 3.0 L of water, and the precipitated grayish white solid was collected by filtration. This solid was dispersed and washed with 1.0 L of methanol and was then dried to produce 97 g of a grayish white solid. This grayish white solid was subjected to silica gel filtration (SiO₂: 400 g, NH—SiO₂: 200 g, developing solvent: toluene at approximately 80° C.) and was then concentrated to produce 96 g of a white solid. This white solid was heated, suspended, and washed with 600 mL of ethanol, was collected by filtration, and was then dried under reduced pressure at 80° C. to produce 86 g of a compound M4 as a white solid (yield: 88%).

### (3) Synthesis of Compound M5

A 2-L recovery flask was charged with the following reagent(s) and solvent(s).
Compound M4: 40.0 g (145 mmol)
Bispinacolborane: 10.3 g (319 mmol)
Pd(OAc)₂: 1.60 g (7.23 mmol)
s-phos: 8.90 g (21.8 mmol)
Potassium acetate: 35.6 g (363 mmol)
Toluene: 800 mL

Next, the reaction solution was heated to 110° C. in a nitrogen stream and was stirred at this temperature (110° C.) for 2 hours. After completion of the reaction, the product was cooled to an internal temperature of 55° C., was hot-filtered through Celite, was concentrated, was dispersed and washed twice with 500 mL of methanol, and was dried under reduced pressure to produce 39 g of a white compound M5 (yield: 73%).

### (4) Synthesis of Compound M7

A 2-L recovery flask was charged with the following reagent(s) and solvent(s).
Compound M5: 35 g (95.3 mmol)
Compound M6: 25 g (93.4 mmol)
Pd(PPh₃)₄: 1.07 g (0.93 mmol)
Sodium carbonate: 14.9 g (140 mmol)
Toluene: 500 ml
Ethanol: 250 ml
Water: 250 ml

Next, the reaction solution was heated to 75° C. in a nitrogen stream and was stirred at this temperature (75° C.) for 3 hours. After completion of the reaction, extraction with toluene and water was performed, and 75 g of silica gel was then added to the filtrate. After the filtrate was stirred at room temperature for 1 hour, the silica gel was filtered off, and the filtrate was concentrated. 75 g of toluene and 150 g of heptane were added for recrystallization to the slurry prepared by the concentration. This operation was repeated twice. After drying under reduced pressure, 31 g of a white compound M7 was prepared (yield: 77%).

### (5) Synthesis of Compound M8

A 2-L recovery flask was charged with the following reagent(s) and solvent(s).
Compound M7: 30.0 g (70.1 mmol)
Bispinacolborane: 39.2 g (154 mmol)
Palladium (II) acetate: 0.79 g (3.51 mmol)
SPhos: 4.32 g (10.5 mmol)
Potassium acetate: 17.2 g (175 mmol)
Toluene: 900 mL

Next, the reaction solution was heated to 110° C. in a nitrogen stream and was stirred at this temperature (110° C.) for 4 hours. After completion of the reaction, the product was cooled to an internal temperature of 80° C., was hot-filtered through Celite, was concentrated, was dispersed and washed twice with 500 mL of methanol, and was dried under reduced pressure to produce 27 g of a white compound M8 (yield: 75%).

### (6) Synthesis of Exemplary Compound A1

A 500-ml recovery flask was charged with the following reagent(s) and solvent(s).
Compound M8: 5.00 g (9.62 mmol)
Compound M9: 2.57 g (9.62 mmol)
Pd(OAc)₂: 0.11 g (0.48 mmol)
s-phos: 0.59 g (1.44 mmol)
Tripotassium phosphate: 4.08 g (19.2 mmol)
Toluene: 250 ml
Water: 25 ml

Next, the reaction solution was heated to 100° C. in a nitrogen stream and was stirred at this temperature (100° C.) for 4 hours. After completion of the reaction, 100 ml of methanol was added thereto, and the product was stirred at room temperature for 30 minutes and was filtered. The residue was purified by silica gel column chromatography (chlorobenzene) and was then dispersed and washed with heptane/toluene to produce 5.70 g of a white exemplary compound A1 (yield: 65%).

The exemplary compound A1 was subjected to mass spectrometry with MALDI-TOF-MS (Autoflex LRF manufactured by Bruker).

### [MALDI-TOF-MS]

Measured value: m/z=623 Calculated value: C₄₆H₂₉N₃=623

### Exemplary Embodiments 2 to 26 (Synthesis of Exemplary Compounds)

As shown in Tables 4 to 9, exemplary compounds were synthesized in the same manner as in Exemplary Embodiment 1 except that the raw material M1 of Example 1 was changed to a raw material 1, the raw material M2 was changed to a raw material 2, the raw material M6 was changed to a raw material 3, and the raw material M9 was changed to a raw material 4. Actual values m/z measured by mass spectrometry in the same manner as in Exemplary Embodiment 1 are also shown.

TABLE 4

| | | Raw | Raw | Raw | | |
|---|---|---|---|---|---|---|
| Exemplary | Exemplary | material | material | material | Raw | |
| embodiment | compound | 1 | 2 | 3 | material 4 | m/z |
| 2 | 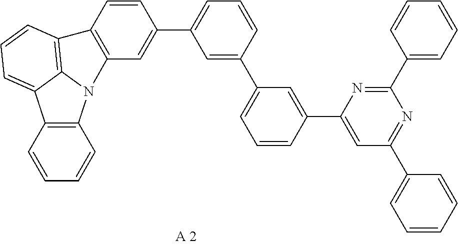 [SMILES: c1ccc(-c2cc(-c3cccc(-c4cccc(-c5ccc6c7cccc8c9ccccc9n(c6c5)c87)c4)c3)nc(-c3ccccc3)n2)cc1] | M1 | M2 | M6 | 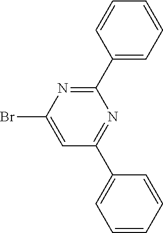 [SMILES: Brc1cc(-c2ccccc2)nc(-c2ccccc2)n1] | 623 |
| 3 | 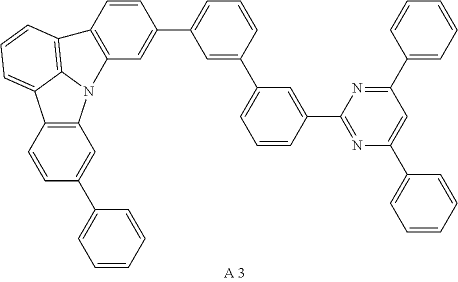 [SMILES: c1ccc(-c2ccc3c4cccc5c6ccc(-c7cccc(-c8cccc(-c9nc(-c%10ccccc%10)cc(-c%10ccccc%10)n9)c8)c7)cc6n(c3c2)c45)cc1] | 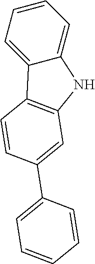 [SMILES: c1ccc(-c2ccc3c(c2)[nH]c2ccccc23)cc1] | M2 | M6 | M9 | 699 |
| 4 | 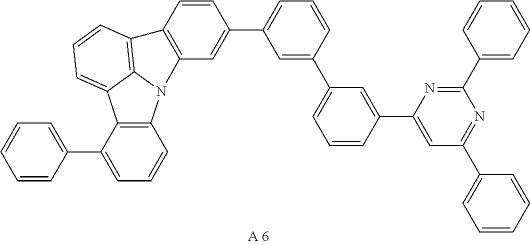 [SMILES: c1ccc(-c2cc(-c3cccc(-c4cccc(-c5ccc6c7cccc8c9c(-c%10ccccc%10)cccc9n(c6c5)c78)c4)c3)nc(-c3ccccc3)n2)cc1] | 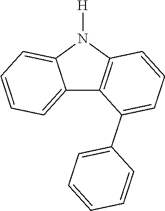 [SMILES: c1ccc(-c2cccc3[nH]c4ccccc4c23)cc1] | M2 | M6 | 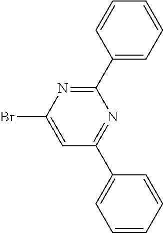 [SMILES: Brc1cc(-c2ccccc2)nc(-c2ccccc2)n1] | 699 |
| 5 | 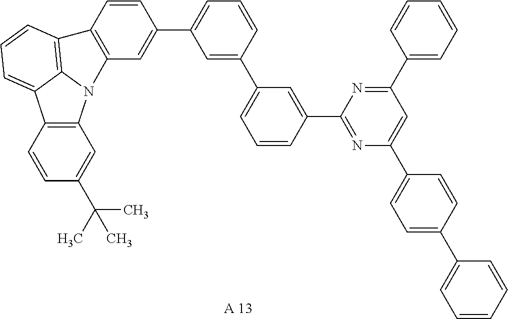 [SMILES: CC(C)(C)c1ccc2c3cccc4c5ccc(-c6cccc(-c7cccc(-c8nc(-c9ccccc9)cc(-c9ccc(-c%10ccccc%10)cc9)n8)c7)c6)cc5n(c2c1)c43] | 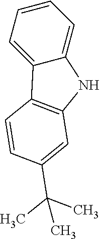 [SMILES: CC(C)(C)c1ccc2c(c1)[nH]c1ccccc12] | M2 | M6 | 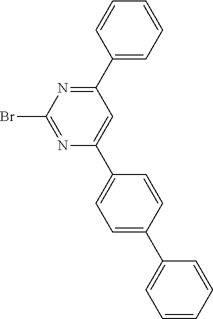 [SMILES: Brc1nc(-c2ccccc2)cc(-c2ccc(-c3ccccc3)cc2)n1] | 755 |
| 6 | 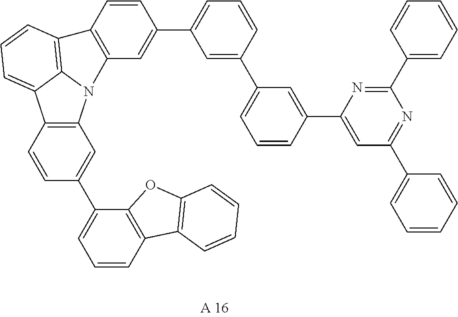 [SMILES: c1ccc(-c2cc(-c3cccc(-c4cccc(-c5ccc6c7cccc8c9ccc(-c%10cccc%11c%10oc%10ccccc%10%11)cc9n(c6c5)c78)c4)c3)nc(-c3ccccc3)n2)cc1] | 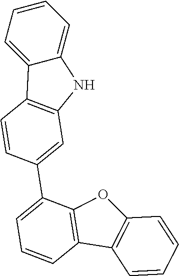 [SMILES: c1ccc2c(c1)[nH]c1cc(-c3cccc4c3oc3ccccc34)ccc12] | M2 | M6 | 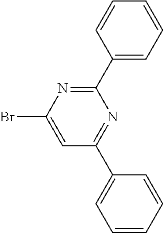 [SMILES: Brc1cc(-c2ccccc2)nc(-c2ccccc2)n1] | 789 |
| 7 | 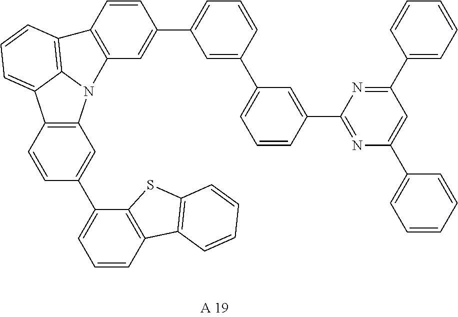 [SMILES: c1ccc(-c2cc(-c3ccccc3)nc(-c3cccc(-c4cccc(-c5ccc6c7cccc8c9ccc(-c%10cccc%11c%10sc%10ccccc%10%11)cc9n(c6c5)c78)c4)c3)n2)cc1] | 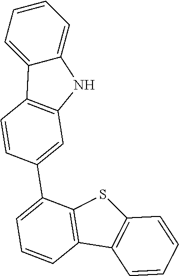 [SMILES: c1ccc2c(c1)[nH]c1cc(-c3cccc4c3sc3ccccc34)ccc12] | M2 | M6 | M9 | 805 |

TABLE 5

| | | Raw | Raw | Raw | Raw | |
|---|---|---|---|---|---|---|
| Exemplary | | material | material | material | material | |
| embodiment | Exemplary compound | 1 | 2 | 3 | 4 | m/z |
| 8 | 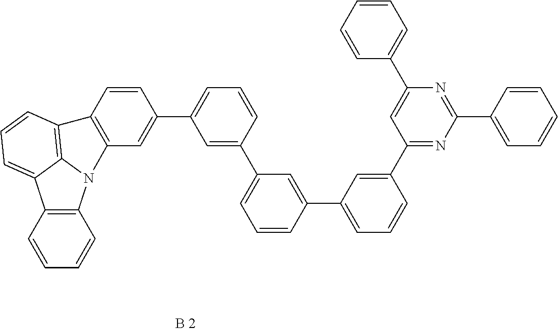 [SMILES: c1ccc(-c2cc(-c3cccc(-c4cccc(-c5cccc(-c6ccc7c8cccc9c%10ccccc%10n(c7c6)c98)c5)c4)c3)nc(-c3ccccc3)n2)cc1] | M1 | M2 | M6 | 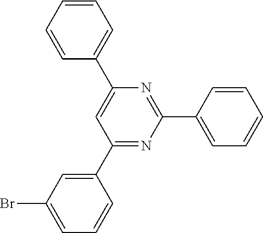 [SMILES: Brc1cccc(-c2cc(-c3ccccc3)nc(-c3ccccc3)n2)c1] | 699 |
| 9 | 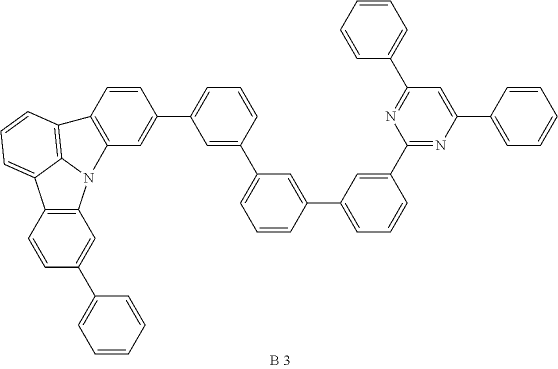 [SMILES: c1ccc(-c2ccc3c4cccc5c6ccc(-c7cccc(-c8cccc(-c9cccc(-c%10nc(-c%11ccccc%11)cc(-c%11ccccc%11)n%10)c9)c8)c7)cc6n(c3c2)c45)cc1] | 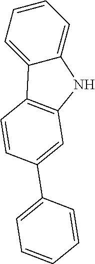 [SMILES: c1ccc(-c2ccc3c(c2)[nH]c2ccccc23)cc1] | M2 | M6 | 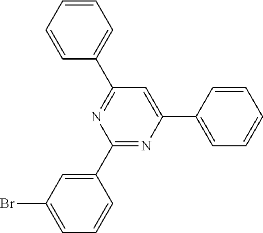 [SMILES: Brc1cccc(-c2nc(-c3ccccc3)cc(-c3ccccc3)n2)c1] | 775 |
| 10 | 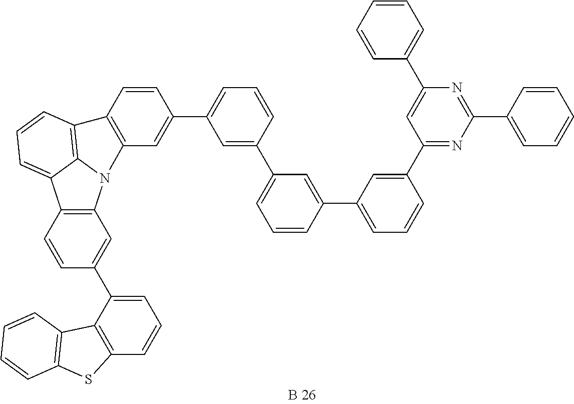 [SMILES: c1ccc(-c2cc(-c3cccc(-c4cccc(-c5cccc(-c6ccc7c8cccc9c%10ccc(-c%11cccc%12sc%13ccccc%13c%11%12)cc%10n(c7c6)c89)c5)c4)c3)nc(-c3ccccc3)n2)cc1] | 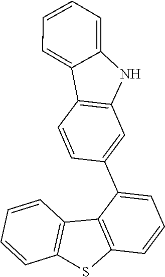 [SMILES: c1ccc2c(c1)[nH]c1cc(-c3cccc4sc5ccccc5c34)ccc12] | M2 | M6 | 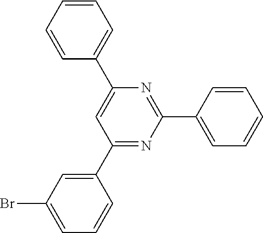 [SMILES: Brc1cccc(-c2cc(-c3ccccc3)nc(-c3ccccc3)n2)c1] | 882 |
| 11 | 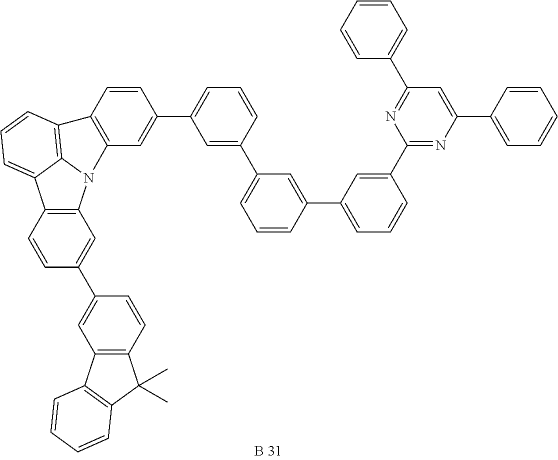 [SMILES: CC1(C)c2ccccc2-c2cc(-c3ccc4c5cccc6c7ccc(-c8cccc(-c9cccc(-c%10cccc(-c%11nc(-c%12ccccc%12)cc(-c%12ccccc%12)n%11)c%10)c9)c8)cc7n(c4c3)c65)ccc21] | 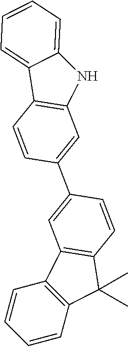 [SMILES: CC1(C)c2ccccc2-c2cc(-c3ccc4c(c3)[nH]c3ccccc34)ccc21] | M2 | M6 | 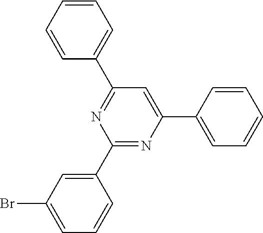 [SMILES: Brc1cccc(-c2nc(-c3ccccc3)cc(-c3ccccc3)n2)c1] | 891 |

TABLE 6

| | | Raw | Raw | Raw | Raw | |
|---|---|---|---|---|---|---|
| Exemplary | | material | material | material | material | |
| embodiment | Exemplary compound | 1 | 2 | 3 | 4 | m/z |
| 12 | 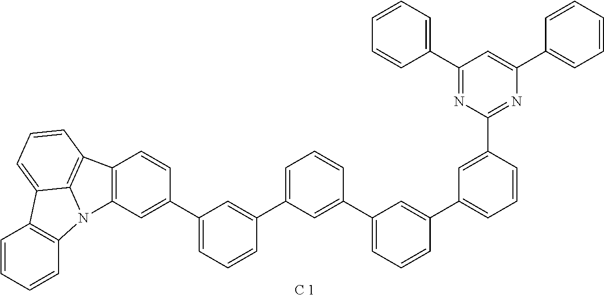 [SMILES: c1ccc(-c2cc(-c3ccccc3)nc(-c3cccc(-c4cccc(-c5cccc(-c6cccc(-c7ccc8c9cccc%10c%11ccccc%11n(c8c7)c%109)c6)c5)c4)c3)n2)cc1] | M1 | M2 | M6 | 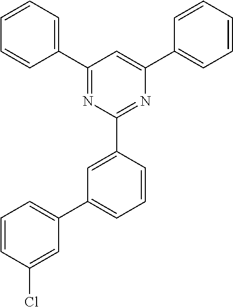 [SMILES: Clc1cccc(-c2cccc(-c3nc(-c4ccccc4)cc(-c4ccccc4)n3)c2)c1] | 775 |
| 13 | 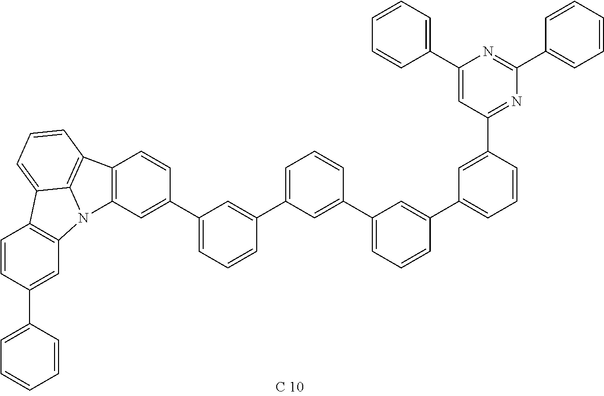 [SMILES: c1ccc(-c2ccc3c4cccc5c6ccc(-c7cccc(-c8cccc(-c9cccc(-c%10cccc(-c%11cc(-c%12ccccc%12)nc(-c%12ccccc%12)n%11)c%10)c9)c8)c7)cc6n(c3c2)c45)cc1] | 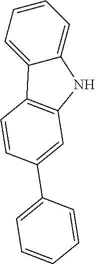 [SMILES: c1ccc(-c2ccc3c(c2)[nH]c2ccccc23)cc1] | M2 | M6 | 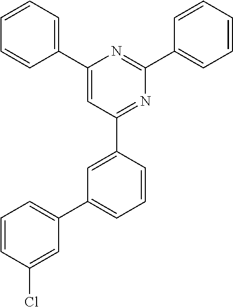 [SMILES: Clc1cccc(-c2cccc(-c3cc(-c4ccccc4)nc(-c4ccccc4)n3)c2)c1] | 851 |
| 14 | 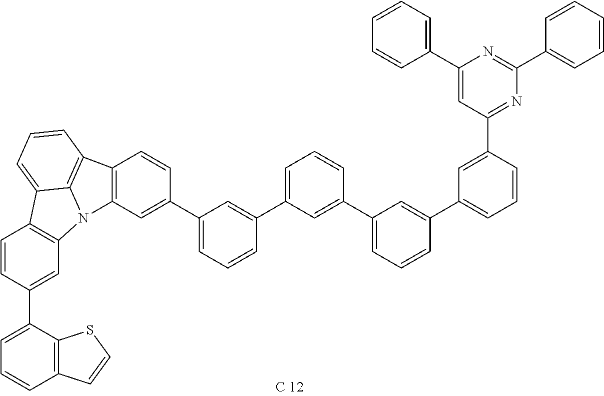 [SMILES: c1ccc(-c2cc(-c3cccc(-c4cccc(-c5cccc(-c6cccc(-c7ccc8c9cccc%10c%11ccc(-c%12cccc%13ccsc%12%13)cc%11n(c8c7)c9%10)c6)c5)c4)c3)nc(-c3ccccc3)n2)cc1] | 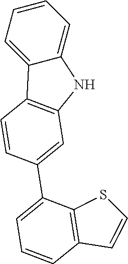 [SMILES: c1cc(-c2ccc3c(c2)[nH]c2ccccc23)c2sccc2c1] | M2 | M6 | 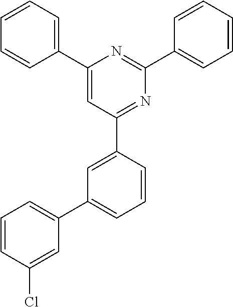 [SMILES: Clc1cccc(-c2cccc(-c3cc(-c4ccccc4)nc(-c4ccccc4)n3)c2)c1] | 907 |

TABLE 7

| | | Raw | Raw | Raw | Raw | |
|---|---|---|---|---|---|---|
| Exemplary | | material | material | material | material | |
| embodiment | Exemplary compound | 1 | 2 | 3 | 4 | m/z |
| 15 | 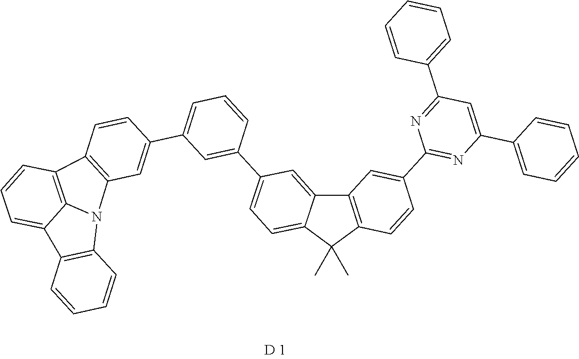 [SMILES: CC1(C)c2ccc(-c3cccc(-c4ccc5c6cccc7c8ccccc8n(c5c4)c76)c3)cc2-c2cc(-c3nc(-c4ccccc4)cc(-c4ccccc4)n3)ccc21] | M1 | M2 | 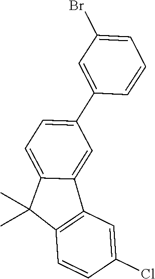 [SMILES: CC1(C)c2ccc(Cl)cc2-c2cc(-c3cccc(Br)c3)ccc21] | M9 | 739 |
| 16 | 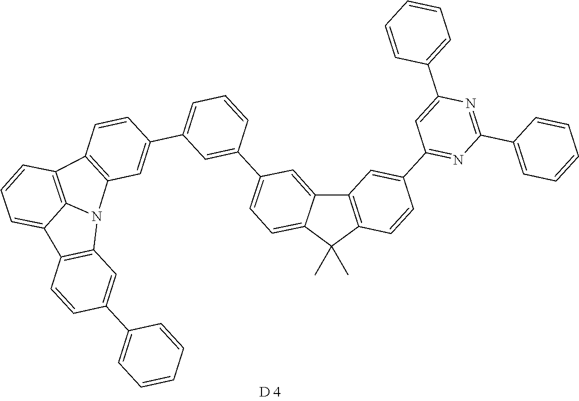 [SMILES: CC1(C)c2ccc(-c3cccc(-c4ccc5c6cccc7c8ccc(-c9ccccc9)cc8n(c5c4)c76)c3)cc2-c2cc(-c3cc(-c4ccccc4)nc(-c4ccccc4)n3)ccc21] | 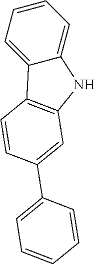 [SMILES: c1ccc(-c2ccc3c(c2)[nH]c2ccccc23)cc1] | M2 | 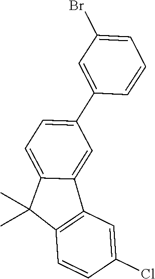 [SMILES: CC1(C)c2ccc(Cl)cc2-c2cc(-c3cccc(Br)c3)ccc21] | 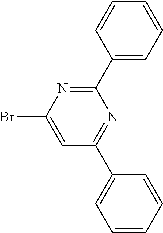 [SMILES: Brc1cc(-c2ccccc2)nc(-c2ccccc2)n1] | 816 |
| 17 | 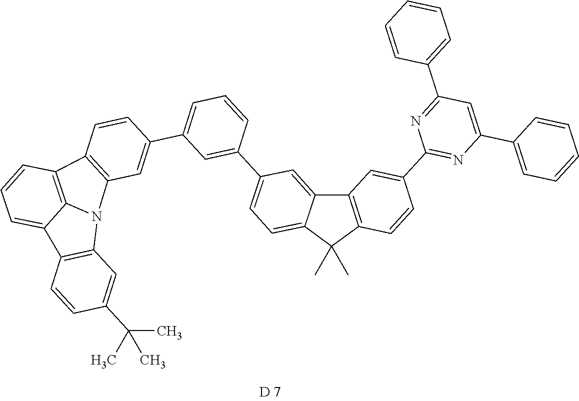 [SMILES: CC(C)(C)c1ccc2c3cccc4c5ccc(-c6cccc(-c7ccc8c(c7)-c7cc(-c9nc(-c%10ccccc%10)cc(-c%10ccccc%10)n9)ccc7C8(C)C)c6)cc5n(c2c1)c43] | 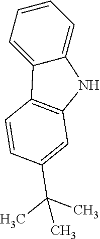 [SMILES: CC(C)(C)c1ccc2c(c1)[nH]c1ccccc12] | M2 | 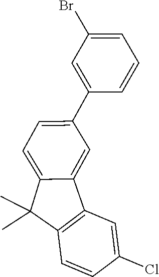 [SMILES: CC1(C)c2ccc(Cl)cc2-c2cc(-c3cccc(Br)c3)ccc21] | M9 | 795 |
| 18 | 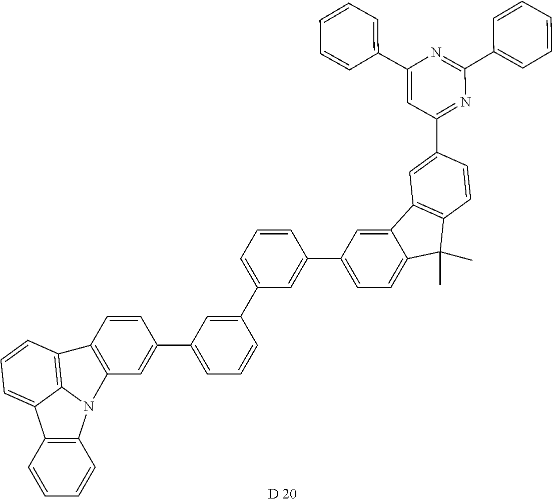 [SMILES: CC1(C)c2ccc(-c3cccc(-c4cccc(-c5ccc6c7cccc8c9ccccc9n(c6c5)c87)c4)c3)cc2-c2cc(-c3cc(-c4ccccc4)nc(-c4ccccc4)n3)ccc21] | M1 | M2 | M6 | 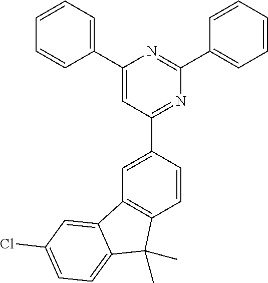 [SMILES: CC1(C)c2ccc(Cl)cc2-c2cc(-c3cc(-c4ccccc4)nc(-c4ccccc4)n3)ccc21] | 815 |

TABLE 8

| Exemplary | | Raw | Raw | Raw | Raw | |
|---|---|---|---|---|---|---|
| embodiment | Exemplary compound | material 1 | material 2 | material 3 | material 4 | m/z |
| 19 | 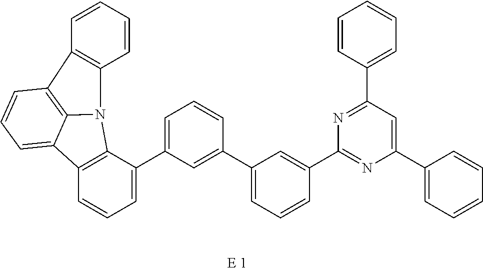 [SMILES: c1ccc(-c2cc(-c3ccccc3)nc(-c3cccc(-c4cccc(-c5cccc6c7cccc8c9ccccc9n(c56)c87)c4)c3)n2)cc1] | M1 | 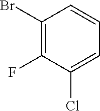 | M6 | M9 | 623 |
| 20 | 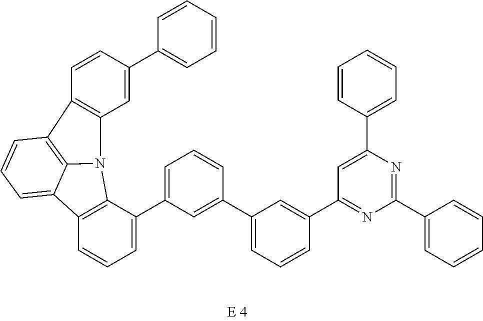 [SMILES: c1ccc(-c2ccc3c4cccc5c6cccc(-c7cccc(-c8cccc(-c9cc(-c%10ccccc%10)nc(-c%10ccccc%10)n9)c8)c7)c6n(c3c2)c45)cc1] | 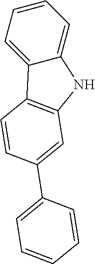 [SMILES: c1ccc(-c2ccc3c(c2)[nH]c2ccccc23)cc1] | 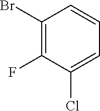 | M6 | 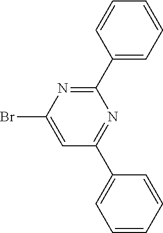 [SMILES: Brc1cc(-c2ccccc2)nc(-c2ccccc2)n1] | 699 |
| 21 | 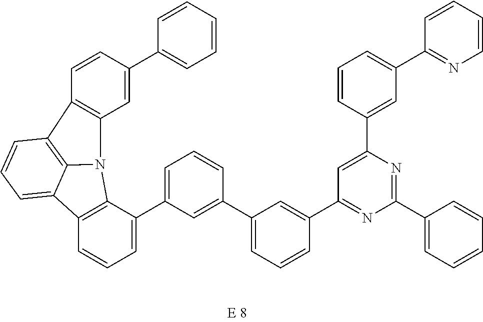 [SMILES: c1ccc(-c2ccc3c4cccc5c6cccc(-c7cccc(-c8cccc(-c9cc(-c%10cccc(-c%11ccccn%11)c%10)nc(-c%10ccccc%10)n9)c8)c7)c6n(c3c2)c45)cc1] | 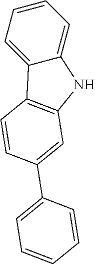 [SMILES: c1ccc(-c2ccc3c(c2)[nH]c2ccccc23)cc1] | 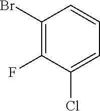 | M6 | 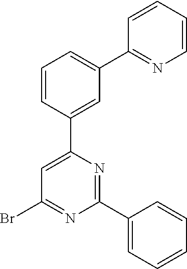 [SMILES: Brc1cc(-c2cccc(-c3ccccn3)c2)nc(-c2ccccc2)n1] | 776 |
| 22 | 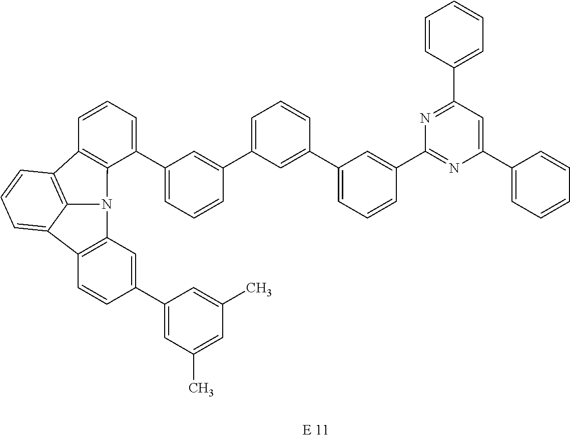 [SMILES: Cc1cc(C)cc(-c2ccc3c4cccc5c6cccc(-c7cccc(-c8cccc(-c9cccc(-c%10nc(-c%11ccccc%11)cc(-c%11ccccc%11)n%10)c9)c8)c7)c6n(c3c2)c45)c1] | 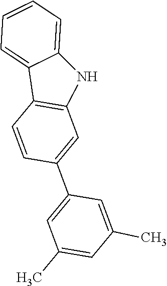 [SMILES: Cc1cc(C)cc(-c2ccc3c(c2)[nH]c2ccccc23)c1] | 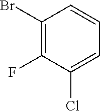 | M6 | 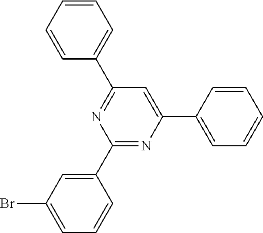 [SMILES: Brc1cccc(-c2nc(-c3ccccc3)cc(-c3ccccc3)n2)c1] | 803 |
| 23 | 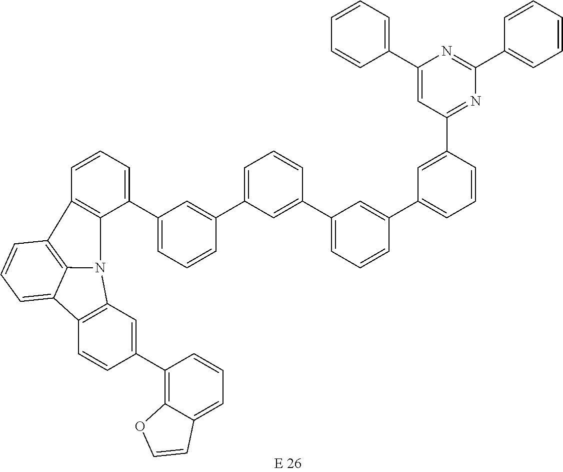 [SMILES: c1ccc(-c2cc(-c3cccc(-c4cccc(-c5cccc(-c6cccc(-c7cccc8c9cccc%10c%11ccc(-c%12cccc%13ccoc%12%13)cc%11n(c78)c%109)c6)c5)c4)c3)nc(-c3ccccc3)n2)cc1] | 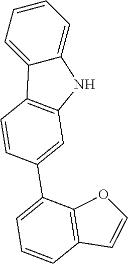 [SMILES: c1cc(-c2ccc3c(c2)[nH]c2ccccc23)c2occc2c1] | 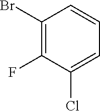 | M6 | 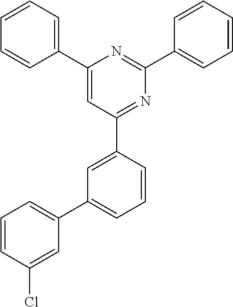 [SMILES: Clc1cccc(-c2cccc(-c3cc(-c4ccccc4)nc(-c4ccccc4)n3)c2)c1] | 891 |

TABLE 9

| | | Raw | Raw | Raw | Raw | |
|---|---|---|---|---|---|---|
| Exemplary | | material | material | material | material | |
| embodiment | Exemplary compound | 1 | 2 | 3 | 4 | m/z |
| 24 | 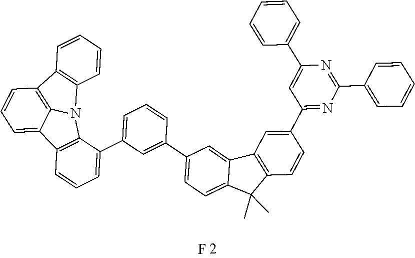 [SMILES: CC1(C)c2ccc(-c3cccc(-c4cccc5c6cccc7c8ccccc8n(c45)c76)c3)cc2-c2cc(-c3cc(-c4ccccc4)nc(-c4ccccc4)n3)ccc21] | M1 | 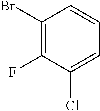 | 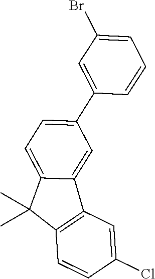 [SMILES: CC1(C)c2ccc(Cl)cc2-c2cc(-c3cccc(Br)c3)ccc21] | 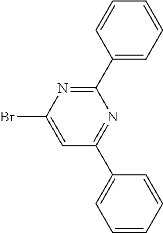 [SMILES: Brc1cc(-c2ccccc2)nc(-c2ccccc2)n1] | 739 |
| 25 | 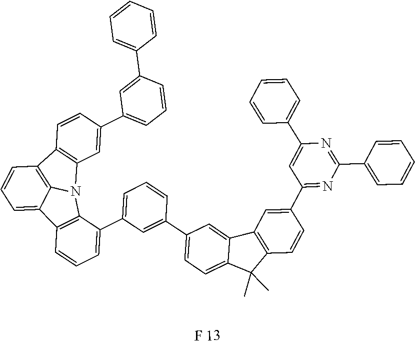 [SMILES: CC1(C)c2ccc(-c3cccc(-c4cccc5c6cccc7c8ccc(-c9cccc(-c%10ccccc%10)c9)cc8n(c45)c76)c3)cc2-c2cc(-c3cc(-c4ccccc4)nc(-c4ccccc4)n3)ccc21] | 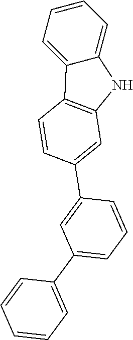 [SMILES: c1ccc(-c2cccc(-c3ccc4c(c3)[nH]c3ccccc34)c2)cc1] | 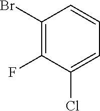 | 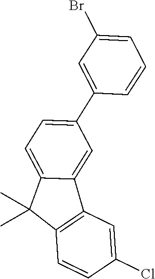 [SMILES: CC1(C)c2ccc(Cl)cc2-c2cc(-c3cccc(Br)c3)ccc21] | M9 | 891 |
| 26 | 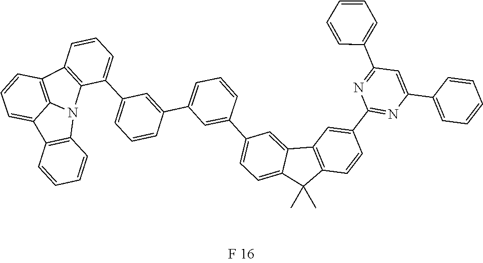 [SMILES: CC1(C)c2ccc(-c3cccc(-c4cccc(-c5cccc6c7cccc8c9ccccc9n(c56)c87)c4)c3)cc2-c2cc(-c3nc(-c4ccccc4)cc(-c4ccccc4)n3)ccc21] | M1 | 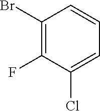 | M6 | 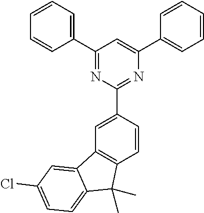 [SMILES: CC1(C)c2ccc(Cl)cc2-c2cc(-c3nc(-c4ccccc4)cc(-c4ccccc4)n3)ccc21] | 815 |

### Exemplary Embodiment 27

An organic light-emitting device of a bottom emission type was produced. The organic light-emitting device included a positive electrode, a hole injection layer, a hole transport layer, an electron-blocking layer, a light-emitting layer, a hole-blocking layer, an electron transport layer, an electron injection layer, and a negative electrode sequentially formed on a substrate.

First, an ITO film was formed on a glass substrate and was subjected to desired patterning to form an ITO electrode (positive electrode). The ITO electrode had a thickness of 100 nm. The substrate on which the ITO electrode was formed was used as an ITO substrate in the following process. Vacuum evaporation was then performed by resistance heating in a vacuum chamber at 1.33×10⁻⁴ Pa to continuously form an organic compound layer and an electrode layer shown in Table 10 on the ITO substrate. The counter electrode (a metal electrode layer, a negative electrode) had an electrode area of 3 mm².

TABLE 10

| | | | | Film |
|---|---|---|---|---|
| | | | | thickness |
| | Material | | | (nm) |
| Negative electrode | Al | | | 100 |
| Electron injection layer | LiF | | | 1 |
| (EIL) | | | | |
| Electron transport layer | ET2 | | | 20 |
| (ETL) | | | | |
| Hole-blocking layer | ET11 | | | 20 |
| (HBL) | | | | |
| Light-emitting layer | | | | |
| (EML) | Host | A3 | Mass ratio | |
| | Guest | GD11 | A3:GD11 = 90:10 | 20 |
| Electron-blocking layer | HT19 | | | 15 |
| (EBL) | | | | |
| Hole transport layer | HT3 | | | 30 |
| (HTL) | | | | |

| | | |
|---|---|---|
| Hole injection layer (HIL) | HT16 | 5 |

The characteristics of the device were measured and evaluated. The light-emitting device had a maximum external quantum efficiency (E.Q.E.) of 13%. A continuous operation test was performed at a current density of 100 mA/cm² to measure the time when the luminance decay rate reached 5%. The present exemplary embodiment had a luminance decay rate ratio of 1.3 on the assumption that the time when the luminance decay rate of Comparative Example 2 reached 5% was 1.0. Table 11 shows these results.

In the present exemplary embodiment, the current-voltage characteristics were measured with a microammeter 4140B manufactured by Hewlett-Packard Co., and the luminous brightness was measured with a BM7 manufactured by Topcon Corporation.

### Exemplary Embodiments 28 to 41 and Comparative Examples 1 to 3

Organic light-emitting devices were produced in the same manner as in Exemplary Embodiment 27 except that the compounds shown in Table 11 were used. Characteristics of the devices were measured and evaluated in the same manner as in Exemplary Embodiment 27. Table 11 shows the measurement results.

TABLE 11

| | | | | EML | | | | E.Q.E | Luminance decay |
|---|---|---|---|---|---|---|---|---|---|
| | HIL | HTL | EBL | Host | Guest | HBL | ETL | [%] | rate ratio |
| Exemplary | HT16 | HT3 | HT19 | A3 | GD11 | ET11 | ET2 | 13 | 1.3 |
| embodiment 27 | | | | | | | | | |
| Exemplary | HT16 | HT3 | HT19 | A1 | GD11 | ET11 | ET2 | 12 | 1.2 |
| embodiment 28 | | | | | | | | | |
| Exemplary | HT16 | HT3 | HT19 | A2 | GD11 | ET11 | ET7 | 12 | 1.2 |
| embodiment 29 | | | | | | | | | |
| Exemplary | HT16 | HT2 | HT19 | A13 | GD11 | ET11 | ET2 | 13 | 1.3 |
| embodiment 30 | | | | | | | | | |
| Exemplary | HT16 | HT3 | HT19 | A19 | GD11 | ET10 | ET2 | 12 | 1.3 |
| embodiment 31 | | | | | | | | | |
| Exemplary | HT16 | HT15 | HT19 | B2 | GD11 | ET10 | ET2 | 13 | 1.2 |
| embodiment 32 | | | | | | | | | |
| Exemplary | HT16 | HT3 | HT19 | B3 | GD11 | ET10 | ET2 | 13 | 1.3 |
| embodiment 33 | | | | | | | | | |
| Exemplary | HT16 | HT15 | HT19 | C1 | GD11 | ET11 | ET2 | 13 | 1.2 |
| embodiment 34 | | | | | | | | | |
| Exemplary | HT16 | HT2 | HT19 | C10 | GD11 | ET12 | ET7 | 13 | 1.2 |
| embodiment 35 | | | | | | | | | |
| Exemplary | HT16 | HT3 | HT19 | D1 | GD11 | ET11 | ET7 | 13 | 1.4 |
| embodiment 36 | | | | | | | | | |
| Exemplary | HT16 | HT3 | HT19 | D4 | GD11 | ET12 | ET2 | 13 | 1.4 |
| embodiment 37 | | | | | | | | | |
| Exemplary | HT16 | HT15 | HT19 | D20 | GD11 | ET12 | ET7 | 13 | 1.3 |
| embodiment 38 | | | | | | | | | |
| Exemplary | HT16 | HT15 | HT19 | E1 | GD11 | ET12 | ET7 | 14 | 1.2 |
| embodiment 39 | | | | | | | | | |
| Exemplary | HT16 | HT3 | HT19 | E4 | GD11 | ET11 | ET2 | 14 | 1.3 |
| embodiment 40 | | | | | | | | | |
| Exemplary | HT16 | HT3 | HT19 | F2 | GD11 | ET11 | ET2 | 14 | 1.3 |
| embodiment 41 | | | | | | | | | |
| Comparative | HT16 | HT3 | HT19 | Comparative | GD11 | ET11 | ET2 | 10 | 0.8 |
| example 1 | | | | compound 1-a | | | | | |
| Comparative | HT16 | HT3 | HT19 | Comparative | GD11 | ET11 | ET2 | 11 | 1.0 |
| example 2 | | | | compound 1-b | | | | | |
| Comparative | HT16 | HT3 | HT19 | Comparative | GD11 | ET11 | ET2 | 13 | 0.9 |
| example 3 | | | | compound 1-c | | | | | |

Table 11 shows that Comparative Examples 1 and 2 had a maximum external quantum efficiency (E.Q.E.) of 10% and 11%, respectively, and the light-emitting devices according to the present embodiments had higher light emission efficiency. This is because the exemplary compounds according to the present embodiments have high T₁ as described in the characteristic (1-1). Furthermore, the light-emitting devices according to the present embodiments had longer device lifetime than the comparative examples. This is because the organic compounds according to the present embodiments satisfy the characteristics (1-2) to (1-4) and improve the durability.

### Exemplary Embodiment 42

An organic light-emitting device was produced in the same manner as in Exemplary Embodiment 27 except that the organic compound layer and the electrode layer shown in Table 12 were continuously formed.

TABLE 12

| | | | | Film |
|---|---|---|---|---|
| | | | | thickness |
| | Material | | | (nm) |
| Negative electrode | Al | | | 100 |
| Electron injection | LiF | | | 1 |
| layer (EIL) | | | | |
| Electron transport | ET2 | | | 20 |
| layer (ETL) | | | | |
| Hole-blocking layer | ET10 | | | 20 |
| (HBL) | | | | |
| Light-emitting layer | | | | |
| (EML) | Host | B3 | Mass ratio | |
| | Guest | GD11 | B3:GD11:EM14 = | 20 |
| | Assist | EM14 | 60:10:30 | |
| Electron-blocking | HT19 | | | 15 |
| layer (EBL) | | | | |
| Hole transport layer | HT3 | | | 30 |
| (HTL) | | | | |
| Hole injection layer | HT16 | | | 5 |
| (HIL) | | | | |

Characteristics of the device was measured and evaluated in the same manner as in Exemplary Embodiment 27. The light-emitting device had a maximum external quantum efficiency (E.Q.E.) of 15%. The present exemplary embodiment had a luminance decay rate ratio of 1.2 on the assumption that the time when the luminance decay rate of Example 33 reached 5% was 1.0. Table 13 shows the results. Table 13 also shows the results of Exemplary Embodiment 33.

### Exemplary Embodiments 43 to 48

Organic light-emitting devices were produced in the same manner as in Exemplary Embodiment 42 except that the compounds shown in Table 13 were used. Characteristics of the devices were measured and evaluated in the same manner as in Exemplary Embodiment 42. Table 13 shows the measurement results. For comparison, Table 13 also shows the results of Exemplary Embodiment 33.

TABLE 13

| | | | | | | | | | | Luminance |
|---|---|---|---|---|---|---|---|---|---|---|
| | | | | EML | | | | | E.Q.E | decay rate |
| | HIL | HTL | EBL | Host | Guest | Assist | HBL | ETL | [%] | ratio |
| Exemplary | HT16 | HT3 | HT19 | B3 | GD11 | EM14 | ET10 | ET2 | 15 | 1.2 |
| embodiment | | | | | | | | | | |
| 42 | | | | | | | | | | |
| Exemplary | HT16 | HT3 | HT19 | A3 | GD11 | EM33 | ET11 | ET2 | 14 | 1.2 |
| embodiment | | | | | | | | | | |
| 43 | | | | | | | | | | |
| Exemplary | HT16 | HT3 | HT19 | A16 | GD11 | EM13 | ET11 | ET7 | 14 | 1.1 |
| embodiment | | | | | | | | | | |
| 44 | | | | | | | | | | |
| Exemplary | HT16 | HT3 | HT19 | B31 | GD11 | EM14 | ET10 | ET2 | 15 | 1.1 |
| embodiment | | | | | | | | | | |
| 45 | | | | | | | | | | |
| Exemplary | HT16 | HT3 | HT19 | D4 | GD11 | EM13 | ET11 | ET7 | 15 | 1.2 |
| embodiment | | | | | | | | | | |
| 46 | | | | | | | | | | |
| Exemplary | HT16 | HT3 | HT19 | D7 | GD11 | EM33 | ET12 | ET2 | 14 | 1.2 |
| embodiment | | | | | | | | | | |
| 47 | | | | | | | | | | |
| Exemplary | HT16 | HT3 | HT19 | E4 | GD11 | EM13 | ET11 | ET2 | 16 | 1.1 |
| embodiment | | | | | | | | | | |
| 48 | | | | | | | | | | |
| Exemplary | HT16 | HT3 | HT19 | B3 | GD11 | — | ET10 | ET2 | 13 | 1.0 |
| embodiment | | | | | | | | | | |
| 33 | | | | | | | | | | |

As shown in Table 13, the compounds according to the present embodiments used together with the assist materials suppress the leakage of holes and electrons to the peripheral layer of the light-emitting layer, thereby improving the maximum external quantum efficiency, and eliminate the carrier accumulation at the interface between the light-emitting layer and the peripheral layer, thereby improving the durability.

Thus, the organic compounds according to the present embodiments can be used to provide light-emitting devices with high light emission efficiency and long device lifetime.

According to the present disclosure, an organic compound according to the present disclosure can be used for an organic light-emitting device to provide the organic light-emitting device with long device lifetime.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-197267 filed Nov. 21, 2023, which is hereby incorporated by reference herein in its entirety.

What is claimed is:

1. An organic compound represented by formula [1] or [2]: 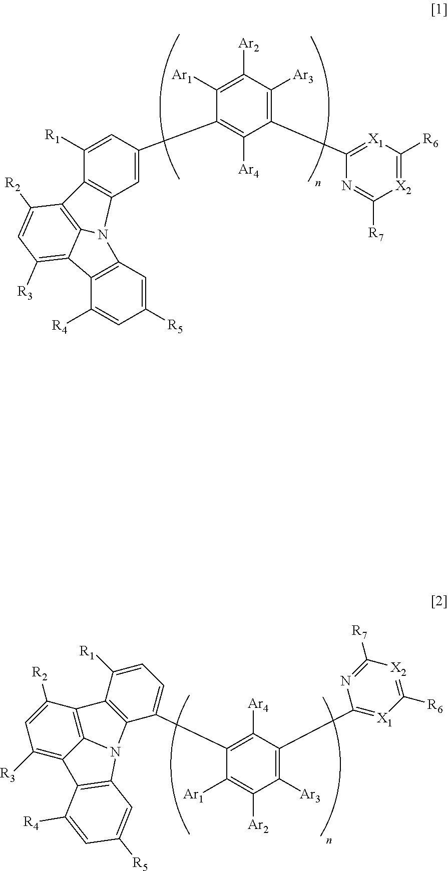
wherein R₁ to R₇ are each independently selected from the group consisting of a hydrogen atom, a deuterium atom, a halogen atom, a substituted or unsubstituted alkyl group, a substituted or unsubstituted aryl group, a substituted or unsubstituted heterocyclic group, a substituted or unsubstituted silyl group, a substituted or unsubstituted amino group, a cyano group, and a substituted or unsubstituted alkoxy group,
Ar₁ to Ar₄ are each independently selected from the group consisting of a hydrogen atom, a deuterium atom, a substituted or unsubstituted alkyl group, a substituted or unsubstituted aryl group, and a substituted or unsubstituted heterocyclic group,
one of x₁ and x₂ is a N atom, and the other is a C atom, and
n is an integer of 2 or more, and adjacent phenylene groups may be bonded together to form a ring.

2. The organic compound according to claim 1, wherein at least one of R₁ to R₅ is a group selected from the group consisting of a deuterium atom, a substituted or unsubstituted alkyl group, a substituted or unsubstituted aryl group, and a substituted or unsubstituted heterocyclic group.

3. The organic compound according to claim 2, wherein at least one of R₃ to R₅ is a group selected from the group consisting of a deuterium atom, a substituted or unsubstituted alkyl group, a substituted or unsubstituted aryl group, and a substituted or unsubstituted heterocyclic group.

4. The organic compound according to claim 3, wherein R₅ is a group selected from the group consisting of a deuterium atom, a substituted or unsubstituted alkyl group, a substituted or unsubstituted aryl group, and a substituted or unsubstituted heterocyclic group.

5. The organic compound according to claim 1, represented by formula [3]: 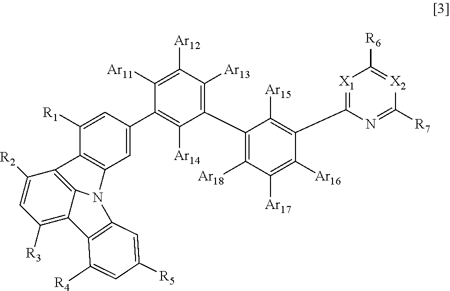
wherein Ar₁₁ to Ar₁₈ are each independently selected from the group consisting of a hydrogen atom, a deuterium atom, a substituted or unsubstituted aryl group, and a substituted or unsubstituted heterocyclic group.

6. The organic compound according to claim 1, represented by formula [4]: 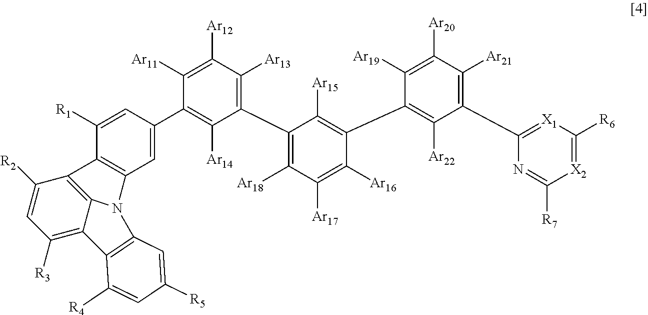
wherein Ar₁₁ to Ar₂₂ are each independently selected from the group consisting of a hydrogen atom, a deuterium atom, a substituted or unsubstituted aryl group, and a substituted or unsubstituted heterocyclic group.

7. The organic compound according to claim 1, represented by formula [5]: 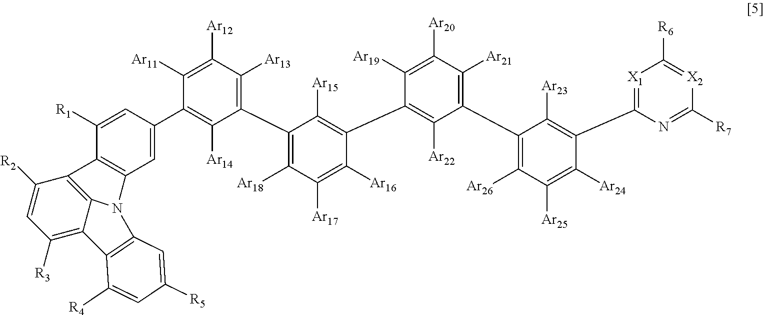
wherein Ar₁₁ to Ar₂₆ are each independently selected from the group consisting of a hydrogen atom, a deuterium atom, a substituted or unsubstituted aryl group, and a substituted or unsubstituted heterocyclic group.

8. The organic compound according to claim 1, represented by formula [6]: 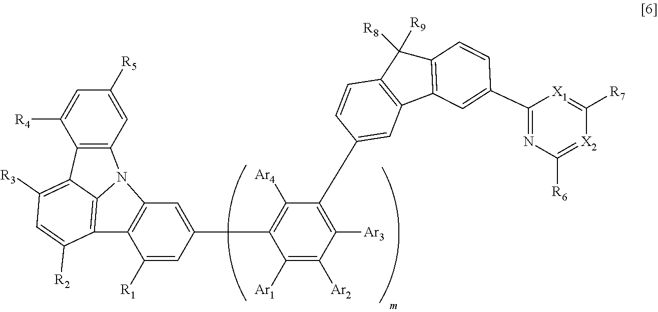
wherein R₈ and R₉ are each independently selected from the group consisting of a hydrogen atom, a deuterium atom, a substituted or unsubstituted alkyl group, a substituted or unsubstituted aryl group, and a substituted or unsubstituted heterocyclic group, and
m is 1 or 2.

9. The organic compound according to claim 1, represented by formula [7]: 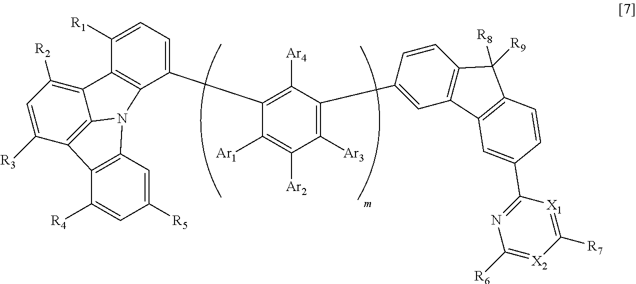
wherein R₈ and R₉ are each independently selected from the group consisting of a hydrogen atom, a deuterium atom, a substituted or unsubstituted alkyl group, a substituted or unsubstituted aryl group, and a substituted or unsubstituted heterocyclic group, and
m is 1 or 2.

10. An organic light-emitting device comprising:
a first electrode;
a second electrode; and
one or more organic compound layers between the first electrode and the second electrode, wherein at least one of the organic compound layers contains the organic compound according to claim 1.

11. The organic light-emitting device according to claim 10, wherein the organic compound layer includes a light-emitting layer, and the light-emitting layer contains the organic compound.

12. The organic light-emitting device according to claim 11, wherein the light-emitting layer further contains a phosphorescent compound.

13. The organic light-emitting device according to claim 12, wherein the light-emitting layer further contains a hole transport compound.

14. The organic light-emitting device according to claim 11, further comprising another light-emitting layer on the light-emitting layer, wherein the other light-emitting layer emits light of a different color from the light-emitting layer.

15. The organic light-emitting device according to claim 14, which emits white light.

16. A display apparatus comprising a plurality of pixels, wherein at least one of the plurality of pixels includes the organic light-emitting device according to claim 10 and a transistor coupled to the organic light-emitting device.

17. A photoelectric conversion apparatus comprising:
an optical unit including a plurality of lenses;
an imaging device configured to receive light passing through the optical unit; and
a display unit configured to display an image taken by the imaging device,
wherein the display unit includes the organic light-emitting device according to claim 10.

18. Electronic equipment comprising:
a display unit including the organic light-emitting device according to claim 10;
a housing configured to be provided with the display unit; and
a communication unit provided in the housing and configured to communicate with an outside.

19. A lighting apparatus comprising:
a light source including the organic light-emitting device according to claim 10; and
a light-diffusing unit or an optical filter configured to transmit light emitted by the light source.

20. A moving body comprising:
a lamp including the organic light-emitting device according to claim 10; and
a body configured to be provided with the lamp.

21. An image-forming apparatus comprising:
a photosensitive member; and
an exposure light source configured to expose the photosensitive member,
wherein the exposure light source includes the organic light-emitting device according to claim 10.
